SEQUENCE LISTING (280 sequences)

SEQ ID NO: 1 - protein, 5 aa, synthetic construct
  REGION 1..5: note = Synthetic - portion of engineered antibody
NYYLN

SEQ ID NO: 2 - protein, 5 aa, synthetic construct
  REGION 1..5: note = Synthetic - portion of engineered antibody
NYYMN

SEQ ID NO: 3 - protein, 5 aa, synthetic construct
  REGION 1..5: note = Synthetic - portion of engineered antibody
NYYMS

SEQ ID NO: 4 - protein, 5 aa, synthetic construct
  REGION 1..5: note = Synthetic - portion of engineered antibody
NYYIN

SEQ ID NO: 5 - protein, 19 aa, synthetic construct
  REGION 1..19: note = Synthetic - portion of engineered antibody
DIRLKSNNYEKHYAESVKG

SEQ ID NO: 6 - protein, 17 aa, synthetic construct
  REGION 1..17: note = Synthetic - portion of engineered antibody
YISSSSSTIYYADSVKG

SEQ ID NO: 7 - protein, 19 aa, synthetic construct
  REGION 1..19: note = Synthetic - portion of engineered antibody
RIKSKTDGGTTDYAAPVKG

SEQ ID NO: 8 - protein, 17 aa, synthetic construct
  REGION 1..17: note = Synthetic - portion of engineered antibody
RIIPILGIANYAQKFQG

SEQ ID NO: 9 - protein, 7 aa, synthetic construct
  REGION 1..7: note = Synthetic - portion of engineered antibody
EGDYFDY

SEQ ID NO: 10 - protein, 118 aa, synthetic construct
  REGION 1..118: note = Synthetic - portion of engineered antibody
EVQLVESGGGLVQPGGSLRLSCAASGFTFSNYYLNWVRQAPGKGLEWVGDIRLKSNNYEKHYAESVKGRFTISRDDSKNSLYLQMNSLKTEDTAVYYCAREGDYFDYWGQGTLVTVSS

SEQ ID NO: 11 - protein, 116 aa, synthetic construct
  REGION 1..116: note = Synthetic - portion of engineered antibody
EVQLVESGGGLVQPGGSLRLSCAASGFTFSNYYMNWVRQAPGKGLEWVSYISSSSSTIYYADSVKGRFTISRDDSKNSLYLQMNSLKTEDTAVYYCAREGDYFDYWGQGTLVTVSS

SEQ ID NO: 12 - protein, 118 aa, synthetic construct
  REGION 1..118: note = Synthetic - portion of engineered antibody
EVQLVESGGGLVQPGGSLRLSCAASGFTFSNYYMNWVRQAPGKGLEWVGRIKSKTDGGTTDYAAPVKGRFTISRDDSKNSLYLQMNSLKTEDTAVYYCAREGDYFDYWGQGTLVTVSS

SEQ ID NO: 13 - protein, 116 aa, synthetic construct
  REGION 1..116: note = Synthetic - portion of engineered antibody
EVQLVESGGGLVQPGGSLRLSCAASGFTFSNYYMNWVRQAPGQGLEWMGRIIPILGIANYAQKFQGRFTISRDDSKNSLYLQMNSLKTEDTAVYYCAREGDYFDYWGQGTLVTVSS

SEQ ID NO: 14 - protein, 118 aa, synthetic construct
  REGION 1..118: note = Synthetic - portion of engineered antibody
EVQLVESGGGLVQPGGSLRLSCAASGFTFSNYYMSWVRQAPGKGLEWVGDIRLKSNNYEKHYAESVKGRFTISRDDSKNSLYLQMNSLKTEDTAVYYCAREGDYFDYWGQGTLVTVSS

SEQ ID NO: 15 - protein, 118 aa, synthetic construct
  REGION 1..118: note = Synthetic - portion of engineered antibody
EVQLVESGGGLVQPGGSLRLSCAASGFTFSNYYINWVRQAPGKGLEWVGDIRLKSNNYEKHYAESVKGRFTISRDDSKNSLYLQMNSLKTEDTAVYYCAREGDYFDYWGQGTLVTVSS

SEQ ID NO: 16 - protein, 15 aa, synthetic construct
  REGION 1..15: note = Synthetic - portion of engineered antibody
KASESVDSFGATFMH

SEQ ID NO: 17 - protein, 15 aa, synthetic construct
  REGION 1..15: note = Synthetic - portion of engineered antibody
RSSESVDSFGATFMH

SEQ ID NO: 18 - protein, 15 aa, synthetic construct
  REGION 1..15: note = Synthetic - portion of engineered antibody
RASESVDSFGATFMH

SEQ ID NO: 19 - protein, 15 aa, synthetic construct
  REGION 1..15: note = Synthetic - portion of engineered antibody
RASQSVSSFGATFMH

SEQ ID NO: 20 - protein, 15 aa, synthetic construct
  REGION 1..15: note = Synthetic - portion of engineered antibody
KASQSVSSFGATFMH

SEQ ID NO: 21 - protein, 7 aa, synthetic construct
  REGION 1..7: note = Synthetic - portion of engineered antibody
LASNLES

SEQ ID NO: 22 - protein, 7 aa, synthetic construct
  REGION 1..7: note = Synthetic - portion of engineered antibody
LASNRAS

SEQ ID NO: 23 - protein, 9 aa, synthetic construct
  REGION 1..9: note = Synthetic - portion of engineered antibody
QQNNEDPYT

SEQ ID NO: 24 - protein, 111 aa, synthetic construct
  REGION 1..111: note = Synthetic - portion of engineered antibody
DIVLTQSPASLAVSPGQRATITCKASESVDSFGATFMHWYQQKPGQPPKLLIYLASNLESGVPARFSGSGSGTDFTLTINPVEADDTANYYCQQNNEDPYTFGQGTKLEIK

SEQ ID NO: 25 - protein, 111 aa, synthetic construct
  REGION 1..111: note = Synthetic - portion of engineered antibody
DIVMTQSPLSLPVTPGEPASISCRSSESVDSFGATFMHWYQQKPGQPPKLLIYLASNRASGVPDRFSGSGSGTDFTLKISRVEAEDVGVYYCQQNNEDPYTFGQGTKLEIK

SEQ ID NO: 26 - protein, 111 aa, synthetic construct
  REGION 1..111: note = Synthetic - portion of engineered antibody
DIVMTQSPLSLPVTPGEPASISCRSSESVDSFGATFMHWYLQKPGQSPQLLIYLASNRASGVPDRFSGSGSGTDFTLKISRVEAEDVGVYYCQQNNEDPYTFGQGTKLEIK

SEQ ID NO: 27 - protein, 111 aa, synthetic construct
  REGION 1..111: note = Synthetic - portion of engineered antibody
EIVLTQSPGTLSLSPGERATLSCRASESVDSFGATFMHWYLQKPGQSPQLLIYLASNRASGVPDRFSGSGSGTDFTLKISRVEAEDVGVYYCQQNNEDPYTFGQGTKLEIK

SEQ ID NO: 28 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLTINPVEADDTANYYCQQNNEDPYTFGQGTKLEIK

SEQ ID NO: 29 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCQQNNEDPYTFGQGTKLEIK

SEQ ID NO: 30 - protein, 111 aa, synthetic construct
  REGION 1..111: note = Synthetic - portion of engineered antibody
DIVLTQSPASLAVSPGQRATITCKASQSVSSFGATFMHWYQQKPGQPPKLLIYLASNRASGVPDRFSGSGSGTDFTLTINPVEADDTANYYCQQNNEDPYTFGQGTKLEIK

SEQ ID NO: 31 - protein, 111 aa, synthetic construct
  REGION 1..111: note = Synthetic - portion of engineered antibody
DIVLTQSPASLAVSPGQRATITCKASQSVSSFGATFMHWYLQKPGQSPQLLIYLASNRASGVPDRFSGSGSGTDFTLTINPVEADDTANYYCQQNNEDPYTFGQGTKLEIK

SEQ ID NO: 32 - protein, 111 aa, synthetic construct
  REGION 1..111: note = Synthetic - portion of engineered antibody
DIVLTQSPASLAVSPGQRATITCKASQSVSSFGATFMHWYLQKPGQSPQLLIYLASNRASGVPDRFSGSGSGTDFTLKISRVEAEDVGVYYCQQNNEDPYTFGQGTKLEIK

SEQ ID NO: 33 - protein, 111 aa, synthetic construct
  REGION 1..111: note = Synthetic - portion of engineered antibody
DIVLTQSPASLAVSPGQRATITCKASQSVSSFGATFMHWYQQKPGQPPKLLIYLASNRASGVPDRFSGSGSGTDFTLKISRVEAEDVGVYYCQQNNEDPYTFGQGTKLEIK

SEQ ID NO: 34 - protein, 111 aa, synthetic construct
  REGION 1..111: note = Synthetic - portion of engineered antibody
EIVLTQSPGTLSLSPGERATLSCRASQSVSSFGATFMHWYLQKPGQSPQLLIYLASNRASGVPDRFSGSGSGTDFTLKISRVEAEDVGVYYCQQNNEDPYTFGQGTKLEIK

SEQ ID NO: 35 - protein, 111 aa, synthetic construct
  REGION 1..111: note = Synthetic - portion of engineered antibody
DIVLTQSPASLAVSPGQRATITCKASESVDSFGATFMHWYQQKPGQPPKLLIYLASNRASGVPDRFSGSGSGTDFTLKISRVEAEDVGVYYCQQNNEDPYTFGQGTKLEIK

SEQ ID NO: 36 - protein, 445 aa, synthetic construct
  REGION 1..445: note = Synthetic - portion of engineered antibody
EVQLVESGGGLVQPGGSLRLSCAASGFTFSNYYLNWVRQAPGKGLEWVGDIRLKSNNYEKHYAESVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 443 aa, synthetic construct
  REGION 1..443: note = Synthetic - portion of engineered antibody
EVQLVESGGGLVQPGGSLRLSCAASGFTFSNYYMNWVRQAPGKGLEWVSYISSSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 445 aa, synthetic construct
  REGION 1..445: note = Synthetic - portion of engineered antibody
EVQLVESGGGLVQPGGSLRLSCAASGFTFSNYYMNWVRQAPGKGLEWVGRIKSKTDGGTTDYAAPVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 443 aa, synthetic construct
  REGION 1..443: note = Synthetic - portion of engineered antibody
EVQLVESGGGLVQPGGSLRLSCAASGFTFSNYYMNWVRQAPGQGLEWMGRIIPILGIANYAQKFQGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 445 aa, synthetic construct
  REGION 1..445: note = Synthetic - portion of engineered antibody
EVQLVESGGGLVQPGGSLRLSCAASGFTFSNYYMSWVRQAPGKGLEWVGDIRLKSNNYEKHYAESVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 445 aa, synthetic construct
  REGION 1..445: note = Synthetic - portion of engineered antibody
EVQLVESGGGLVQPGGSLRLSCAASGFTFSNYYINWVRQAPGKGLEWVGDIRLKSNNYEKHYAESVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 218 aa, synthetic construct
  REGION 1..218: note = Synthetic - portion of engineered antibody
DIVLTQSPASLAVSPGQRATITCKASESVDSFGATFMHWYQQKPGQPPKLLIYLASNLESGVPARFSGSGSGTDFTLTINPVEADDTANYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 218 aa, synthetic construct
  REGION 1..218: note = Synthetic - portion of engineered antibody
DIVMTQSPLSLPVTPGEPASISCRSSESVDSFGATFMHWYQQKPGQPPKLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 218 aa, synthetic construct
  REGION 1..218: note = Synthetic - portion of engineered antibody
DIVMTQSPLSLPVTPGEPASISCRSSESVDSFGATFMHWYLQKPGQSPQLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 218 aa, synthetic construct
  REGION 1..218: note = Synthetic - portion of engineered antibody
EIVLTQSPGTLSLSPGERATLSCRASESVDSFGATFMHWYLQKPGQSPQLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLTINPVEADDTANYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 218 aa, synthetic construct
  REGION 1..218: note = Synthetic - portion of engineered antibody
DIVLTQSPASLAVSPGQRATITCKASQSVSSFGATFMHWYQQKPGQPPKLLIYLASNRASGVPDRFSGSGSGTDFTLTINPVEADDTANYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 218 aa, synthetic construct
  REGION 1..218: note = Synthetic - portion of engineered antibody
DIVLTQSPASLAVSPGQRATITCKASQSVSSFGATFMHWYLQKPGQSPQLLIYLASNRASGVPDRFSGSGSGTDFTLTINPVEADDTANYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 218 aa, synthetic construct
  REGION 1..218: note = Synthetic - portion of engineered antibody
DIVLTQSPASLAVSPGQRATITCKASQSVSSFGATFMHWYLQKPGQSPQLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 218 aa, synthetic construct
  REGION 1..218: note = Synthetic - portion of engineered antibody
DIVLTQSPASLAVSPGQRATITCKASQSVSSFGATFMHWYQQKPGQPPKLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 218 aa, synthetic construct
  REGION 1..218: note = Synthetic - portion of engineered antibody
EIVLTQSPGTLSLSPGERATLSCRASQSVSSFGATFMHWYLQKPGQSPQLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 218 aa, synthetic construct
  REGION 1..218: note = Synthetic - portion of engineered antibody
DIVLTQSPASLAVSPGQRATITCKASESVDSFGATFMHWYQQKPGQPPKLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 15 bp, synthetic construct
  misc_feature 1..15: note = Synthetic - portion of engineered antibody
  source 1..15: mol_type = other DNA
aactactacctgaac

SEQ ID NO: 55 - DNA, 15 bp, synthetic construct
  misc_feature 1..15: note = Synthetic - portion of engineered antibody
  source 1..15: mol_type = other DNA
aactactacatgaac

SEQ ID NO: 56 - DNA, 15 bp, synthetic construct
  misc_feature 1..15: note = Synthetic - portion of engineered antibody
  source 1..15: mol_type = other DNA
aactactacatgagc

SEQ ID NO: 57 - DNA, 15 bp, synthetic construct
  misc_feature 1..15: note = Synthetic - portion of engineered antibody
  source 1..15: mol_type = other DNA
aactactacatcaac

SEQ ID NO: 58 - DNA, 57 bp, synthetic construct
  misc_feature 1..57: note = Synthetic - portion of engineered antibody
  source 1..57: mol_type = other DNA
gacatccggctgaagtccaacaactacgagaagcactacgccgagagcgtgaagggc

SEQ ID NO: 59 - DNA, 51 bp, synthetic construct
  misc_feature 1..51: note = Synthetic - portion of engineered antibody
  source 1..51: mol_type = other DNA
tacatcagcagcagctccagcaccatctactacgccgacagcgtgaagggc

SEQ ID NO: 60 - DNA, 57 bp, synthetic construct
  misc_feature 1..57: note = Synthetic - portion of engineered antibody
  source 1..57: mol_type = other DNA
cggatcaagagcaagaccgatggcggcaccaccgattatgctgcccctgtgaagggc

SEQ ID NO: 61 - DNA, 51 bp, synthetic construct
  misc_feature 1..51: note = Synthetic - portion of engineered antibody
  source 1..51: mol_type = other DNA
agaatcatccccatcctgggaatcgccaactacgcccagaagttccagggc

SEQ ID NO: 62 - DNA, 21 bp, synthetic construct
  misc_feature 1..21: note = Synthetic - portion of engineered antibody
  source 1..21: mol_type = other DNA
gagggcgactacttcgactat

SEQ ID NO: 63 - DNA, 354 bp, synthetic construct
  misc_feature 1..354: note = Synthetic - portion of engineered antibody
  source 1..354: mol_type = other DNA
gaagtgcagctggtggaatctggcggaggactggttcaacctggcggctctctgagactgtcttgtgccgccagcggcttcaccttcagcaactactacctgaactgggtccgacaggcccctggcaaaggactggaatgggtcggagacatccggctgaagtccaacaactacgagaagcactacgccgagagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 64 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 65 - DNA, 354 bp, synthetic construct
  misc_feature 1..354: note = Synthetic - portion of engineered antibody
  source 1..354: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtcggacggatcaagagcaagaccgatggcggcaccaccgattatgctgcccctgtgaagggcagattcaccatcagcagggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 66 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgaactgggtccgacaggcccctggacagggacttgaatggatgggcagaatcatccccatcctgggaatcgccaactacgcccagaagttccagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 67 - DNA, 354 bp, synthetic construct
  misc_feature 1..354: note = Synthetic - portion of engineered antibody
  source 1..354: mol_type = other DNA
gaagtgcagctggtggaatctggcggaggactggttcaacctggcggctctctgagactgtcttgtgccgccagcggcttcaccttcagcaactactacatgagctgggtccgacaggcccctggcaaaggacttgagtgggtcggagacatccggctgaagtccaacaactacgagaagcactacgccgagagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 68 - DNA, 354 bp, synthetic construct
  misc_feature 1..354: note = Synthetic - portion of engineered antibody
  source 1..354: mol_type = other DNA
gaagtgcagctggtggaatctggcggaggactggttcaacctggcggctctctgagactgtcttgtgccgccagcggcttcaccttcagcaactactacatcaactgggtccgacaggcccctggcaaaggccttgaatgggtcggagacatccggctgaagtccaacaactacgagaagcactacgccgagagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 69 - DNA, 45 bp, synthetic construct
  misc_feature 1..45: note = Synthetic - portion of engineered antibody
  source 1..45: mol_type = other DNA
aaggccagcgagagcgtggacagctttggcgccaccttcatgcac

SEQ ID NO: 70 - DNA, 45 bp, synthetic construct
  misc_feature 1..45: note = Synthetic - portion of engineered antibody
  source 1..45: mol_type = other DNA
agaagcagcgagagcgtggacagcttcggcgccaccttcatgcac

SEQ ID NO: 71 - DNA, 45 bp, synthetic construct
  misc_feature 1..45: note = Synthetic - portion of engineered antibody
  source 1..45: mol_type = other DNA
agagccagcgagagcgtggactcttttggcgccaccttcatgcac

SEQ ID NO: 72 - DNA, 45 bp, synthetic construct
  misc_feature 1..45: note = Synthetic - portion of engineered antibody
  source 1..45: mol_type = other DNA
agagccagccagagcgttagctctttcggcgccaccttcatgcac

SEQ ID NO: 73 - DNA, 45 bp, synthetic construct
  misc_feature 1..45: note = Synthetic - portion of engineered antibody
  source 1..45: mol_type = other DNA
aaggccagccagagcgtgtccagctttggcgccaccttcatgcac

SEQ ID NO: 74 - DNA, 21 bp, synthetic construct
  misc_feature 1..21: note = Synthetic - portion of engineered antibody
  source 1..21: mol_type = other DNA
ctggcctccaacctggaaagc

SEQ ID NO: 75 - DNA, 21 bp, synthetic construct
  misc_feature 1..21: note = Synthetic - portion of engineered antibody
  source 1..21: mol_type = other DNA
ctggcctccaatagagccagc

SEQ ID NO: 76 - DNA, 21 bp, synthetic construct
  misc_feature 1..21: note = Synthetic - portion of engineered antibody
  source 1..21: mol_type = other DNA
ctggccagcaatagagccagc

SEQ ID NO: 77 - DNA, 21 bp, synthetic construct
  misc_feature 1..21: note = Synthetic - portion of engineered antibody
  source 1..21: mol_type = other DNA
ctggccagcaatagagcctcc

SEQ ID NO: 78 - DNA, 27 bp, synthetic construct
  misc_feature 1..27: note = Synthetic - portion of engineered antibody
  source 1..27: mol_type = other DNA
cagcagaacaacgaggacccctacacc

SEQ ID NO: 79 - DNA, 333 bp, synthetic construct
  misc_feature 1..333: note = Synthetic - portion of engineered antibody
  source 1..333: mol_type = other DNA
gatatcgtgctgacacagagccctgccagtctggctgtttctcctggacagagggccaccatcacatgcaaggccagcgagagcgtggacagctttggcgccaccttcatgcactggtatcagcagaagcctggccagcctcctaagctgctgatctacctggcctccaacctggaaagcggagtgcctgccagattttctggctctggcagcggcaccgacttcaccctgacaatcaatcccgtggaagccgacgacaccgccaactactactgccagcagaacaacgaggacccctacacctttggccagggcaccaagctggaaatcaaa

SEQ ID NO: 80 - DNA, 333 bp, synthetic construct
  misc_feature 1..333: note = Synthetic - portion of engineered antibody
  source 1..333: mol_type = other DNA
gacatcgtgatgacacagagccctctgagcctgcctgtgacacctggcgaacctgccagcatcagctgtagaagcagcgagagcgtggacagcttcggcgccaccttcatgcactggtatcagcagaagcctggccagcctcctaagctgctgatctacctggcctccaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaa

SEQ ID NO: 81 - DNA, 333 bp, synthetic construct
  misc_feature 1..333: note = Synthetic - portion of engineered antibody
  source 1..333: mol_type = other DNA
gacatcgtgatgacacagagccctctgagcctgcctgtgacacctggcgaacctgccagcatcagctgtagaagcagcgagagcgtggacagcttcggcgccaccttcatgcactggtatctgcagaagcccggccagtctcctcagctgctgatctacctggccagcaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaa

SEQ ID NO: 82 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagcgagagcgtggactcttttggcgccaccttcatgcactggtatctgcagaagcccggccagtctcctcagctgctgatctacctggccagcaatagagcctccggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 83 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaccatcaatcccgtggaagccgacgacaccgccaactactactgccagcagaacaacgaggacccctacacctttggccagggcaccaagctggaaatcaaa

SEQ ID NO: 84 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 85 - DNA, 333 bp, synthetic construct
  misc_feature 1..333: note = Synthetic - portion of engineered antibody
  source 1..333: mol_type = other DNA
gatatcgtgctgacacagagccctgccagtctggctgtttctcctggacagagggccaccatcacatgcaaggccagccagagcgtgtccagctttggcgccaccttcatgcactggtatcagcagaagcctggccagcctcctaagctgctgatctacctggcctccaatagagccagcggcgtgcccgatagattttctggctctggcagcggcaccgacttcaccctgacaatcaatcccgtggaagccgacgacaccgccaactactactgccagcagaacaacgaggacccctacacctttggccagggcaccaagctggaaatcaaa

SEQ ID NO: 86 - DNA, 333 bp, synthetic construct
  misc_feature 1..333: note = Synthetic - portion of engineered antibody
  source 1..333: mol_type = other DNA
gatatcgtgctgacacagagccctgccagtctggctgtttctcctggacagagggccaccatcacatgcaaggccagccagagcgtgtccagctttggcgccaccttcatgcactggtatctgcagaagcccgggcagtctcctcagctgctgatctacctggccagcaatagagccagcggcgtgcccgatagattttctggctctggcagcggcaccgacttcaccctgacaatcaatcccgtggaagccgacgacaccgccaactactactgccagcagaacaacgaggacccctacacctttggccagggcaccaagctggaaatcaaa

SEQ ID NO: 87 - DNA, 333 bp, synthetic construct
  misc_feature 1..333: note = Synthetic - portion of engineered antibody
  source 1..333: mol_type = other DNA
gatatcgtgctgacacagagccctgccagtctggctgtttctcctggacagagggccaccatcacatgcaaggccagccagagcgtgtccagctttggcgccaccttcatgcactggtatctgcagaagcccgggcagtctcctcagctgctgatctacctggccagcaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaa

SEQ ID NO: 88 - DNA, 333 bp, synthetic construct
  misc_feature 1..333: note = Synthetic - portion of engineered antibody
  source 1..333: mol_type = other DNA
gatatcgtgctgacacagagccctgccagtctggctgtttctcctggacagagggccaccatcacatgcaaggccagccagagcgtgtccagctttggcgccaccttcatgcactggtatcagcagaagcctggccagcctcctaagctgctgatctacctggcctccaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaa

SEQ ID NO: 89 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatctgcagaagcccggccagtctcctcagctgctgatctacctggccagcaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 90 - DNA, 333 bp, synthetic construct
  misc_feature 1..333: note = Synthetic - portion of engineered antibody
  source 1..333: mol_type = other DNA
gatatcgtgctgacacagagccctgccagtctggctgtttctcctggacagagggccaccatcacatgcaaggccagcgagagcgtggacagctttggcgccaccttcatgcactggtatcagcagaagcctggccagcctcctaagctgctgatctacctggcctccaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaa

SEQ ID NO: 91 - DNA, 1335 bp, synthetic construct
  misc_feature 1..1335: note = Synthetic - portion of engineered antibody
  source 1..1335: mol_type = other DNA
gaagtgcagctggtggaatctggcggaggactggttcaacctggcggctctctgagactgtcttgtgccgccagcggcttcaccttcagcaactactacctgaactgggtccgacaggcccctggcaaaggactggaatgggtcggagacatccggctgaagtccaacaactacgagaagcactacgccgagagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 92 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 93 - DNA, 1335 bp, synthetic construct
  misc_feature 1..1335: note = Synthetic - portion of engineered antibody
  source 1..1335: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtcggacggatcaagagcaagaccgatggcggcaccaccgattatgctgcccctgtgaagggcagattcaccatcagcagggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 94 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgaactgggtccgacaggcccctggacagggacttgaatggatgggcagaatcatccccatcctgggaatcgccaactacgcccagaagttccagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 95 - DNA, 1335 bp, synthetic construct
  misc_feature 1..1335: note = Synthetic - portion of engineered antibody
  source 1..1335: mol_type = other DNA
gaagtgcagctggtggaatctggcggaggactggttcaacctggcggctctctgagactgtcttgtgccgccagcggcttcaccttcagcaactactacatgagctgggtccgacaggcccctggcaaaggacttgagtgggtcggagacatccggctgaagtccaacaactacgagaagcactacgccgagagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 96 - DNA, 1335 bp, synthetic construct
  misc_feature 1..1335: note = Synthetic - portion of engineered antibody
  source 1..1335: mol_type = other DNA
gaagtgcagctggtggaatctggcggaggactggttcaacctggcggctctctgagactgtcttgtgccgccagcggcttcaccttcagcaactactacatcaactgggtccgacaggcccctggcaaaggccttgaatgggtcggagacatccggctgaagtccaacaactacgagaagcactacgccgagagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 97 - DNA, 654 bp, synthetic construct
  misc_feature 1..654: note = Synthetic - portion of engineered antibody
  source 1..654: mol_type = other DNA
gatatcgtgctgacacagagccctgccagtctggctgtttctcctggacagagggccaccatcacatgcaaggccagcgagagcgtggacagctttggcgccaccttcatgcactggtatcagcagaagcctggccagcctcctaagctgctgatctacctggcctccaacctggaaagcggagtgcctgccagattttctggctctggcagcggcaccgacttcaccctgacaatcaatcccgtggaagccgacgacaccgccaactactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 98 - DNA, 654 bp, synthetic construct
  misc_feature 1..654: note = Synthetic - portion of engineered antibody
  source 1..654: mol_type = other DNA
gacatcgtgatgacacagagccctctgagcctgcctgtgacacctggcgaacctgccagcatcagctgtagaagcagcgagagcgtggacagcttcggcgccaccttcatgcactggtatcagcagaagcctggccagcctcctaagctgctgatctacctggcctccaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 99 - DNA, 654 bp, synthetic construct
  misc_feature 1..654: note = Synthetic - portion of engineered antibody
  source 1..654: mol_type = other DNA
gacatcgtgatgacacagagccctctgagcctgcctgtgacacctggcgaacctgccagcatcagctgtagaagcagcgagagcgtggacagcttcggcgccaccttcatgcactggtatctgcagaagcccggccagtctcctcagctgctgatctacctggccagcaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 100 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagcgagagcgtggactcttttggcgccaccttcatgcactggtatctgcagaagcccggccagtctcctcagctgctgatctacctggccagcaatagagcctccggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 101 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaccatcaatcccgtggaagccgacgacaccgccaactactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 102 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 103 - DNA, 654 bp, synthetic construct
  misc_feature 1..654: note = Synthetic - portion of engineered antibody
  source 1..654: mol_type = other DNA
gatatcgtgctgacacagagccctgccagtctggctgtttctcctggacagagggccaccatcacatgcaaggccagccagagcgtgtccagctttggcgccaccttcatgcactggtatcagcagaagcctggccagcctcctaagctgctgatctacctggcctccaatagagccagcggcgtgcccgatagattttctggctctggcagcggcaccgacttcaccctgacaatcaatcccgtggaagccgacgacaccgccaactactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 104 - DNA, 654 bp, synthetic construct
  misc_feature 1..654: note = Synthetic - portion of engineered antibody
  source 1..654: mol_type = other DNA
gatatcgtgctgacacagagccctgccagtctggctgtttctcctggacagagggccaccatcacatgcaaggccagccagagcgtgtccagctttggcgccaccttcatgcactggtatctgcagaagcccgggcagtctcctcagctgctgatctacctggccagcaatagagccagcggcgtgcccgatagattttctggctctggcagcggcaccgacttcaccctgacaatcaatcccgtggaagccgacgacaccgccaactactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 105 - DNA, 654 bp, synthetic construct
  misc_feature 1..654: note = Synthetic - portion of engineered antibody
  source 1..654: mol_type = other DNA
gatatcgtgctgacacagagccctgccagtctggctgtttctcctggacagagggccaccatcacatgcaaggccagccagagcgtgtccagctttggcgccaccttcatgcactggtatctgcagaagcccgggcagtctcctcagctgctgatctacctggccagcaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 106 - DNA, 654 bp, synthetic construct
  misc_feature 1..654: note = Synthetic - portion of engineered antibody
  source 1..654: mol_type = other DNA
gatatcgtgctgacacagagccctgccagtctggctgtttctcctggacagagggccaccatcacatgcaaggccagccagagcgtgtccagctttggcgccaccttcatgcactggtatcagcagaagcctggccagcctcctaagctgctgatctacctggcctccaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 107 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatctgcagaagcccggccagtctcctcagctgctgatctacctggccagcaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 108 - DNA, 654 bp, synthetic construct
  misc_feature 1..654: note = Synthetic - portion of engineered antibody
  source 1..654: mol_type = other DNA
gatatcgtgctgacacagagccctgccagtctggctgtttctcctggacagagggccaccatcacatgcaaggccagcgagagcgtggacagctttggcgccaccttcatgcactggtatcagcagaagcctggccagcctcctaagctgctgatctacctggcctccaatagagccagcggcgtgcccgatagattttctggctctggcagcggcaccgacttcaccctgaagatctctagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 109 - protein, 5 aa, synthetic construct
  REGION 1..5: note = Synthetic - portion of engineered antibody
IYYMN

SEQ ID NO: 110 - protein, 5 aa, synthetic construct
  REGION 1..5: note = Synthetic - portion of engineered antibody
NFYMN

SEQ ID NO: 111 - protein, 5 aa, synthetic construct
  REGION 1..5: note = Synthetic - portion of engineered antibody
PYYMN

SEQ ID NO: 112 - protein, 5 aa, synthetic construct
  REGION 1..5: note = Synthetic - portion of engineered antibody
FYYMN

SEQ ID NO: 113 - protein, 5 aa, synthetic construct
  REGION 1..5: note = Synthetic - portion of engineered antibody
NYYFN

SEQ ID NO: 114 - protein, 17 aa, synthetic construct
  REGION 1..17: note = Synthetic - portion of engineered antibody
YISTSSSTIYYADSVKG

SEQ ID NO: 115 - protein, 17 aa, synthetic construct
  REGION 1..17: note = Synthetic - portion of engineered antibody
YISSRSSTIYYADSVKG

SEQ ID NO: 116 - protein, 17 aa, synthetic construct
  REGION 1..17: note = Synthetic - portion of engineered antibody
YISSSSRTIYYADSVKG

SEQ ID NO: 117 - protein, 17 aa, synthetic construct
  REGION 1..17: note = Synthetic - portion of engineered antibody
YISSSSSPIYYADSVKG

SEQ ID NO: 118 - protein, 7 aa, synthetic construct
  REGION 1..7: note = Synthetic - portion of engineered antibody
EGDYMDY

SEQ ID NO: 119 - protein, 116 aa, synthetic construct
  REGION 1..116: note = Synthetic - portion of engineered antibody
EVQLVESGGGLVQPGGSLRLSCAASGFTFSNYYMNWVRQAPGKGLEWVSYISTSSSTIYYADSVKGRFTISRDDSKNSLYLQMNSLKTEDTAVYYCAREGDYFDYWGQGTLVTVSS

SEQ ID NO: 120 - protein, 116 aa, synthetic construct
  REGION 1..116: note = Synthetic - portion of engineered antibody
EVQLVESGGGLVQPGGSLRLSCAASGFTFSIYYMNWVRQAPGKGLEWVSYISSSSSTIYYADSVKGRFTISRDDSKNSLYLQMNSLKTEDTAVYYCAREGDYFDYWGQGTLVTVSS

SEQ ID NO: 121 - protein, 116 aa, synthetic construct
  REGION 1..116: note = Synthetic - portion of engineered antibody
EVQLVESGGGLVQPGGSLRLSCAASGFTFSNFYMNWVRQAPGKGLEWVSYISSSSSTIYYADSVKGRFTISRDDSKNSLYLQMNSLKTEDTAVYYCAREGDYFDYWGQGTLVTVSS

SEQ ID NO: 122 - protein, 116 aa, synthetic construct
  REGION 1..116: note = Synthetic - portion of engineered antibody
EVQLVESGGGLVQPGGSLRLSCAASGFTFSPYYMNWVRQAPGKGLEWVSYISSSSSTIYYADSVKGRFTISRDDSKNSLYLQMNSLKTEDTAVYYCAREGDYFDYWGQGTLVTVSS

SEQ ID NO: 123 - protein, 116 aa, synthetic construct
  REGION 1..116: note = Synthetic - portion of engineered antibody
EVQLVESGGGLVQPGGSLRLSCAASGFTFSNYYMSWVRQAPGKGLEWVSYISSSSSTIYYADSVKGRFTISRDDSKNSLYLQMNSLKTEDTAVYYCAREGDYFDYWGQGTLVTVSS

SEQ ID NO: 124 - protein, 116 aa, synthetic construct
  REGION 1..116: note = Synthetic - portion of engineered antibody
EVQLVESGGGLVQPGGSLRLSCAASGFTFSFYYMNWVRQAPGKGLEWVSYISSSSSTIYYADSVKGRFTISRDDSKNSLYLQMNSLKTEDTAVYYCAREGDYFDYWGQGTLVTVSS

SEQ ID NO: 125 - protein, 116 aa, synthetic construct
  REGION 1..116: note = Synthetic - portion of engineered antibody
EVQLVESGGGLVQPGGSLRLSCAASGFTFSNYYMNWVRQAPGKGLEWVSYISSSSSTIYYADSVKGRFTISRDDSKNSLYLQMNSLKTEDTAVYYCAREGDYMDYWGQGTLVTVSS

SEQ ID NO: 126 - protein, 116 aa, synthetic construct
  REGION 1..116: note = Synthetic - portion of engineered antibody
EVQLVESGGGLVQPGGSLRLSCAASGFTFSNYYMNWVRQAPGKGLEWVSYISSRSSTIYYADSVKGRFTISRDDSKNSLYLQMNSLKTEDTAVYYCAREGDYFDYWGQGTLVTVSS

SEQ ID NO: 127 - protein, 116 aa, synthetic construct
  REGION 1..116: note = Synthetic - portion of engineered antibody
EVQLVESGGGLVQPGGSLRLSCAASGFTFSNYYMNWVRQAPGKGLEWVSYISSSSRTIYYADSVKGRFTISRDDSKNSLYLQMNSLKTEDTAVYYCAREGDYFDYWGQGTLVTVSS

SEQ ID NO: 128 - protein, 116 aa, synthetic construct
  REGION 1..116: note = Synthetic - portion of engineered antibody
EVQLVESGGGLVQPGGSLRLSCAASGFTFSNYYFNWVRQAPGKGLEWVSYISSSSSTIYYADSVKGRFTISRDDSKNSLYLQMNSLKTEDTAVYYCAREGDYFDYWGQGTLVTVSS

SEQ ID NO: 129 - protein, 116 aa, synthetic construct
  REGION 1..116: note = Synthetic - portion of engineered antibody
EVQLVESGGGLVQPGGSLRLSCAASGFTFSNYYMNWVRQAPGKGLEWVSYISSSSSPIYYADSVKGRFTISRDDSKNSLYLQMNSLKTEDTAVYYCAREGDYFDYWGQGTLVTVSS

SEQ ID NO: 130 - protein, 15 aa, synthetic construct
  REGION 1..15: note = Synthetic - portion of engineered antibody
RASESVSSFGATFMH

SEQ ID NO: 131 - protein, 15 aa, synthetic construct
  REGION 1..15: note = Synthetic - portion of engineered antibody
RASQRVSSFGATFMH

SEQ ID NO: 132 - protein, 7 aa, synthetic construct
  REGION 1..7: note = Synthetic - portion of engineered antibody
LAWNRAS

SEQ ID NO: 133 - protein, 7 aa, synthetic construct
  REGION 1..7: note = Synthetic - portion of engineered antibody
LASWRAS

SEQ ID NO: 134 - protein, 7 aa, synthetic construct
  REGION 1..7: note = Synthetic - portion of engineered antibody
LKSNRAS

SEQ ID NO: 135 - protein, 7 aa, synthetic construct
  REGION 1..7: note = Synthetic - portion of engineered antibody
LASHRAS

SEQ ID NO: 136 - protein, 7 aa, synthetic construct
  REGION 1..7: note = Synthetic - portion of engineered antibody
LARNRAS

SEQ ID NO: 137 - protein, 7 aa, synthetic construct
  REGION 1..7: note = Synthetic - portion of engineered antibody
LASRRAS

SEQ ID NO: 138 - protein, 9 aa, synthetic construct
  REGION 1..9: note = Synthetic - portion of engineered antibody
QQNNEDPFT

SEQ ID NO: 139 - protein, 9 aa, synthetic construct
  REGION 1..9: note = Synthetic - portion of engineered antibody
QQNNEDPWT

SEQ ID NO: 140 - protein, 9 aa, synthetic construct
  REGION 1..9: note = Synthetic - portion of engineered antibody
TQNNEDPYT

SEQ ID NO: 141 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCQQNNEDPFTFGQGTKLEIK

SEQ ID NO: 142 - protein, 111 aa, synthetic construct
  REGION 1..111: note = Synthetic - portion of engineered antibody
EIVLTQSPGTLSLSPGERATLSCRASESVSSFGATFMHWYQQKPGQPPKLLIYLASNRASGVPDRFSGSGSGTDFTLKISRVEAEDVGVYYCQQNNEDPYTFGQGTKLEIK

SEQ ID NO: 143 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLAWNRASGVPDRFSGSGSGTDFTLKISRVEAEDVGVYYCQQNNEDPYTFGQGTKLEIK

SEQ ID NO: 144 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASWRASGVPDRFSGSGSGTDFTLKISRVEAEDVGVYYCQQNNEDPYTFGQGTKLEIK

SEQ ID NO: 145 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLKSNRASGVPDRFSGSGSGTDFTLKISRVEAEDVGVYYCQQNNEDPYTFGQGTKLEIK

SEQ ID NO: 146 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASHRASGVPDRFSGSGSGTDFTLKISRVEAEDVGVYYCQQNNEDPYTFGQGTKLEIK

SEQ ID NO: 147 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCQQNNEDPWTFGQGTKLEIK

SEQ ID NO: 148 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLARNRASGVPDRFSGSGSGTDFTLKISRVEAEDVGVYYCQQNNEDPYTFGQGTKLEIK

SEQ ID NO: 149 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCTQNNEDPYTFGQGTKLEIK

SEQ ID NO: 150 - protein, 111 aa, synthetic construct
  REGION 1..111: note = Synthetic - portion of engineered antibody
EIVLTQSPGTLSLSPGERATLSCRASQRVSSFGATFMHWYQQKPGQPPKLLIYLASNRASGVPDRFSGSGSGTDFTLKISRVEAEDVGVYYCQQNNEDPYTFGQGTKLEIK

SEQ ID NO: 151 - protein, 111 aa, synthetic construct
  REGION 1..111: note = Synthetic - portion of engineered antibody
EIVLTQSPGTLSLSPGERATLSCRASQSVSSFGATFMHWYQQKPGQPPKLLIYLASRRASGVPDRFSGSGSGTDFTLKISRVEAEDVGVYYCQQNNEDPYTFGQGTKLEIK

SEQ ID NO: 152 - protein, 443 aa, synthetic construct
  REGION 1..443: note = Synthetic - portion of engineered antibody
EVQLVESGGGLVQPGGSLRLSCAASGFTFSNYYMNWVRQAPGKGLEWVSYISTSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 153 - protein, 443 aa, synthetic construct
  REGION 1..443: note = Synthetic - portion of engineered antibody
EVQLVESGGGLVQPGGSLRLSCAASGFTFSIYYMNWVRQAPGKGLEWVSYISSSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 154 - protein, 443 aa, synthetic construct
  REGION 1..443: note = Synthetic - portion of engineered antibody
EVQLVESGGGLVQPGGSLRLSCAASGFTFSNFYMNWVRQAPGKGLEWVSYISSSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 155 - protein, 443 aa, synthetic construct
  REGION 1..443: note = Synthetic - portion of engineered antibody
EVQLVESGGGLVQPGGSLRLSCAASGFTFSPYYMNWVRQAPGKGLEWVSYISSSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - protein, 443 aa, synthetic construct
  REGION 1..443: note = Synthetic - portion of engineered antibody
EVQLVESGGGLVQPGGSLRLSCAASGFTFSNYYMSWVRQAPGKGLEWVSYISSSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - protein, 443 aa, synthetic construct
  REGION 1..443: note = Synthetic - portion of engineered antibody
EVQLVESGGGLVQPGGSLRLSCAASGFTFSFYYMNWVRQAPGKGLEWVSYISSSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 443 aa, synthetic construct
  REGION 1..443: note = Synthetic - portion of engineered antibody
EVQLVESGGGLVQPGGSLRLSCAASGFTFSNYYMNWVRQAPGKGLEWVSYISSSSSTIYYADSVKGRFTISRDDSKNSLYLQMNSLKTEDTAVYYCAREGDYMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 443 aa, synthetic construct
  REGION 1..443: note = Synthetic - portion of engineered antibody
EVQLVESGGGLVQPGGSLRLSCAASGFTFSNYYMNWVRQAPGKGLEWVSYISSRSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - protein, 443 aa, synthetic construct
  REGION 1..443: note = Synthetic - portion of engineered antibody
EVQLVESGGGLVQPGGSLRLSCAASGFTFSNYYMNWVRQAPGKGLEWVSYISSSSRTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 443 aa, synthetic construct
  REGION 1..443: note = Synthetic - portion of engineered antibody
EVQLVESGGGLVQPGGSLRLSCAASGFTFSNYYFNWVRQAPGKGLEWVSYISSSSSTIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 443 aa, synthetic construct
  REGION 1..443: note = Synthetic - portion of engineered antibody
EVQLVESGGGLVQPGGSLRLSCAASGFTFSNYYMNWVRQAPGKGLEWVSYISSSSSPIYYADSVKGRFTISRDDSKNSLYLQMNSLKTEDTAVYYCAREGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 218 aa, synthetic construct
  REGION 1..218: note = Synthetic - portion of engineered antibody
EIVLTQSPGTLSLSPGERATLSCRASESVSSFGATFMHWYQQKPGQPPKLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLAWNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASWRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLKSNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASHRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCQQNNEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLARNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASNRASGVPDRFSGSGSGTDFTLKISRVEAEDVGVYYCTQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 218 aa, synthetic construct
  REGION 1..218: note = Synthetic - portion of engineered antibody
EIVLTQSPGTLSLSPGERATLSCRASQRVSSFGATFMHWYQQKPGQPPKLLIYLASNRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 218 aa, synthetic construct
  REGION 1..218: note = Synthetic - portion of engineered antibody
EIVLTQSPGTLSLSPGERATLSCRASQSVSSFGATFMHWYQQKPGQPPKLLIYLASRRASGVPDRFSGSGSGTDFTLKISRVEAEDVGVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - DNA, 15 bp, synthetic construct
  misc_feature 1..15: note = Synthetic - portion of engineered antibody
  source 1..15: mol_type = other DNA
atctactacatgaac

SEQ ID NO: 175 - DNA, 15 bp, synthetic construct
  misc_feature 1..15: note = Synthetic - portion of engineered antibody
  source 1..15: mol_type = other DNA
aacttctacatgaac

SEQ ID NO: 176 - DNA, 15 bp, synthetic construct
  misc_feature 1..15: note = Synthetic - portion of engineered antibody
  source 1..15: mol_type = other DNA
ccctactacatgaac

SEQ ID NO: 177 - DNA, 15 bp, synthetic construct
  misc_feature 1..15: note = Synthetic - portion of engineered antibody
  source 1..15: mol_type = other DNA
ttctactacatgaac

SEQ ID NO: 178 - DNA, 15 bp, synthetic construct
  misc_feature 1..15: note = Synthetic - portion of engineered antibody
  source 1..15: mol_type = other DNA
aactactacttcaac

SEQ ID NO: 179 - DNA, 51 bp, synthetic construct
  misc_feature 1..51: note = Synthetic - portion of engineered antibody
  source 1..51: mol_type = other DNA
tacatcagcaccagctccagcaccatctactacgccgacagcgtgaagggc

SEQ ID NO: 180 - DNA, 51 bp, synthetic construct
  misc_feature 1..51: note = Synthetic - portion of engineered antibody
  source 1..51: mol_type = other DNA
tacatcagcagcagctccagcacaatctactacgccgacagcgtgaagggc

SEQ ID NO: 181 - DNA, 51 bp, synthetic construct
  misc_feature 1..51: note = Synthetic - portion of engineered antibody
  source 1..51: mol_type = other DNA
tacatcagcagccggtccagcaccatctactacgccgatagcgtgaagggc

SEQ ID NO: 182 - DNA, 51 bp, synthetic construct
  misc_feature 1..51: note = Synthetic - portion of engineered antibody
  source 1..51: mol_type = other DNA
tacatcagcagcagctcccggaccatctactacgccgatagcgtgaagggc

SEQ ID NO: 183 - DNA, 51 bp, synthetic construct
  misc_feature 1..51: note = Synthetic - portion of engineered antibody
  source 1..51: mol_type = other DNA
tacatcagcagcagctctagccccatctactacgccgacagcgtgaagggc

SEQ ID NO: 184 - DNA, 21 bp, synthetic construct
  misc_feature 1..21: note = Synthetic - portion of engineered antibody
  source 1..21: mol_type = other DNA
gaaggcgactacatggactat

SEQ ID NO: 185 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcaccagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 186 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcatctactacatgaactgggtccgacaggcccctggcaaaggccttgaatgggtgtcctacatcagcagcagctccagcacaatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 187 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaacttctacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 188 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcacattcagcccctactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 189 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgagctgggtccgacaggcccctggcaaaggacttgagtgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 190 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcttctactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 191 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgcgccagagaaggcgactacatggactattggggccagggcacactggtcacagtctcttca

SEQ ID NO: 192 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagccggtccagcaccatctactacgccgatagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 193 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctcccggaccatctactacgccgatagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 194 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacttcaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 195 - DNA, 348 bp, synthetic construct
  misc_feature 1..348: note = Synthetic - portion of engineered antibody
  source 1..348: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctctagccccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttca

SEQ ID NO: 196 - DNA, 45 bp, synthetic construct
  misc_feature 1..45: note = Synthetic - portion of engineered antibody
  source 1..45: mol_type = other DNA
agagccagcgagagcgttagctctttcggcgccaccttcatgcac

SEQ ID NO: 197 - DNA, 45 bp, synthetic construct
  misc_feature 1..45: note = Synthetic - portion of engineered antibody
  source 1..45: mol_type = other DNA
agagccagccagagagtgtctagcttcggcgccaccttcatgcac

SEQ ID NO: 198 - DNA, 21 bp, synthetic construct
  misc_feature 1..21: note = Synthetic - portion of engineered antibody
  source 1..21: mol_type = other DNA
ctggcctccaatagagcctcc

SEQ ID NO: 199 - DNA, 21 bp, synthetic construct
  misc_feature 1..21: note = Synthetic - portion of engineered antibody
  source 1..21: mol_type = other DNA
ctggcctggaatagagccagc

SEQ ID NO: 200 - DNA, 21 bp, synthetic construct
  misc_feature 1..21: note = Synthetic - portion of engineered antibody
  source 1..21: mol_type = other DNA
ctggcctcttggagagccagc

SEQ ID NO: 201 - DNA, 21 bp, synthetic construct
  misc_feature 1..21: note = Synthetic - portion of engineered antibody
  source 1..21: mol_type = other DNA
ctgaagtccaatagagccagc

SEQ ID NO: 202 - DNA, 21 bp, synthetic construct
  misc_feature 1..21: note = Synthetic - portion of engineered antibody
  source 1..21: mol_type = other DNA
ctggcctctcacagagcctct

SEQ ID NO: 203 - DNA, 21 bp, synthetic construct
  misc_feature 1..21: note = Synthetic - portion of engineered antibody
  source 1..21: mol_type = other DNA
ctggccagaaatagagccagc

SEQ ID NO: 204 - DNA, 21 bp, synthetic construct
  misc_feature 1..21: note = Synthetic - portion of engineered antibody
  source 1..21: mol_type = other DNA
ctggcttctcggagagccagc

SEQ ID NO: 205 - DNA, 27 bp, synthetic construct
  misc_feature 1..27: note = Synthetic - portion of engineered antibody
  source 1..27: mol_type = other DNA
cagcagaacaacgaggaccccttcacc

SEQ ID NO: 206 - DNA, 27 bp, synthetic construct
  misc_feature 1..27: note = Synthetic - portion of engineered antibody
  source 1..27: mol_type = other DNA
cagcagaacaacgaggacccctggaca

SEQ ID NO: 207 - DNA, 27 bp, synthetic construct
  misc_feature 1..27: note = Synthetic - portion of engineered antibody
  source 1..27: mol_type = other DNA
acccagaacaacgaggacccctacacc

SEQ ID NO: 208 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggaccccttcacctttggccagggcaccaagctggaaatcaaa

SEQ ID NO: 209 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagcgagagcgttagctctttcggcgccaccttcatgcactggtatcagcagaagcctggccagcctcctaagctgctgatctacctggcctccaatagagcctccggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 210 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctggaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 211 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctcttggagagccagcggcgtgccagatagattttctggcagcggctccggcaccgacttcaccctgaagatctctagagtggaagccgaggacgtgggcgtgtactactgccagcagaacaacgaggacccctacacctttggccagggcaccaagctggaaatcaaa

SEQ ID NO: 212 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctgaagtccaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 213 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctctcacagagcctctggcgtgcccgatagattttctggcagcggctctggcaccgacttcaccctgaagatctccagagtggaagccgaggacgtgggcgtgtactactgccagcagaacaacgaggacccctacacctttggccagggcaccaagctggaaatcaaa

SEQ ID NO: 214 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggacccctggacatttggccagggcaccaagctggaaatcaaa

SEQ ID NO: 215 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggccagaaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 216 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgcacccagaacaacgaggacccctacacctttggccagggcaccaagctggaaatcaaa

SEQ ID NO: 217 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagagtgtctagcttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaa

SEQ ID NO: 218 - DNA, 333 bp, synthetic construct
  misc_feature 1..333: note = Synthetic - portion of engineered antibody
  source 1..333: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcttctcggagagccagcggcgtgccagatagattttctggcagcggctccggcaccgacttcaccctgaagatctctagagtggaagccgaggacgtgggcgtgtactactgccagcagaacaacgaggacccctacacctttggccagggcaccaagctggaaatcaaa

SEQ ID NO: 219 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcaccagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 220 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcatctactacatgaactgggtccgacaggcccctggcaaaggccttgaatgggtgtcctacatcagcagcagctccagcacaatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 221 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaacttctacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 222 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcacattcagcccctactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 223 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgagctgggtccgacaggcccctggcaaaggacttgagtgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 224 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcttctactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 225 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgcgccagagaaggcgactacatggactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 226 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagccggtccagcaccatctactacgccgatagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 227 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctcccggaccatctactacgccgatagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 228 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacttcaactgggtccgacaggcccctggcaaaggccttgaatgggtgtcctacatcagcagcagctccagcaccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 229 - DNA, 1329 bp, synthetic construct
  misc_feature 1..1329: note = Synthetic - portion of engineered antibody
  source 1..1329: mol_type = other DNA
gaagtgcagctggtggaatctggcggaggactggttcaacctggcggctctctgagactgtcttgtgccgccagcggcttcaccttcagcaactactacatgaactgggtccgacaggcccctggcaaaggccttgaatgggtgtcctacatcagcagcagctctagccccatctactacgccgacagcgtgaagggcagattcaccatcagccgggacgacagcaagaacagcctgtacctgcagatgaactccctgaaaaccgaggacaccgccgtgtactactgtgccagagagggcgactacttcgactattggggccagggcacactggtcacagtctcttcagccagcaccaagggccccagcgtgttccctctggccccttgtagcagaagcaccagcgagtctacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 230 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggaccccttcacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 231 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagcgagagcgttagctctttcggcgccaccttcatgcactggtatcagcagaagcctggccagcctcctaagctgctgatctacctggcctccaatagagcctccggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 232 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctggaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 233 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctcttggagagccagcggcgtgccagatagattttctggcagcggctccggcaccgacttcaccctgaagatctctagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 234 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctgaagtccaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 235 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctctcacagagcctctggcgtgcccgatagattttctggcagcggctctggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 236 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggacccctggacatttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 237 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggccagaaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 238 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgcacccagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 239 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagagtgtctagcttcggcgccaccttcatgcactggtatcagcagaagcctggccagcctcctaagctgctgatctacctggcctccaatagagccagcggcgtgcccgatagattttctggctctggaagcggcaccgacttcaccctgaagatctccagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 240 - DNA, 654 bp, synthetic construct
  misc_feature 1..654: note = Synthetic - portion of engineered antibody
  source 1..654: mol_type = other DNA
gagattgtgctgacacagagccccggcacactgtcactttctccaggcgaaagagccacactgagctgcagagccagccagagcgttagctctttcggcgccaccttcatgcactggtatcagcagaagcctggccagcctcctaagctgctgatctacctggcttctcggagagccagcggcgtgccagatagattttctggcagcggctccggcaccgacttcaccctgaagatctctagagtggaagccgaggacgtgggcgtgtactactgccagcagaacaacgaggacccctacacctttggccagggcaccaagctggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 241 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 242 - protein, 405 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 243 - protein, 5 aa, synthetic construct
NYYIS

SEQ ID NO: 244 - protein, 5 aa, synthetic construct
NAYMS

SEQ ID NO: 245 - protein, 5 aa, synthetic construct
NAYIN

SEQ ID NO: 246 - protein, 5 aa, synthetic construct
SYYLN

SEQ ID NO: 247 - protein, 5 aa, synthetic construct
SAYMS

SEQ ID NO: 248 - protein, 5 aa, synthetic construct
FYYLN

SEQ ID NO: 249 - protein, 19 aa, synthetic construct
DIRLKKNNYEKHYAESVKG

SEQ ID NO: 250 - protein, 116 aa, synthetic construct
EVQLVESGGGLVQPGGSLRLSCAASGFTFSNYYMSWVRQAPGQGLEWMGRIIPILGIANYAQKFQGRFTISRDDSKNSLYLQMNSLKTEDTAVYYCAREGDYFDYWGQGTLVTVSS

SEQ ID NO: 251 - protein, 116 aa, synthetic construct
EVQLVESGGGLVQPGGSLRLSCAASGFTFSNYYINWVRQAPGKGLEWVSYISSSSSTIYYADSVKGRFTISRDDSKNSLYLQMNSLKTEDTAVYYCAREGDYFDYWGQGTLVTVSS

SEQ ID NO: 252 - protein, 118 aa, synthetic construct
EVQLVESGGGLVQPGGSLRLSCAASGFTFSNYYISWVRQAPGKGLEWVGRIKSKTDGGTTDYAAPVKGRFTISRDDSKNSLYLQMNSLKTEDTAVYYCAREGDYFDYWGQGTLVTVSS

SEQ ID NO: 253 - protein, 118 aa, synthetic construct
EVQLVESGGGLVQPGGSLRLSCAASGFTFSNAYMSWVRQAPGKGLEWVGRIKSKTDGGTTDYAAPVKGRFTISRDDSKNSLYLQMNSLKTEDTAVYYCAREGDYFDYWGQGTLVTVSS

SEQ ID NO: 254 - protein, 116 aa, synthetic construct
EVQLVESGGGLVQPGGSLRLSCAASGFTFSNAYINWVRQAPGKGLEWVSYISSSSSTIYYADSVKGRFTISRDDSKNSLYLQMNSLKTEDTAVYYCAREGDYFDYWGQGTLVTVSS

SEQ ID NO: 255 - protein, 116 aa, synthetic construct
EVQLVESGGGLVQPGGSLRLSCAASGFTFSSYYLNWVRQAPGKGLEWVSYISSSSSTIYYADSVKGRFTISRDDSKNSLYLQMNSLKTEDTAVYYCAREGDYFDYWGQGTLVTVSS

SEQ ID NO: 256 - protein, 116 aa, synthetic construct
EVQLVESGGGLVQPGGSLRLSCAASGFTFSSAYMSWVRQAPGQGLEWMGRIIPILGIANYAQKFQGRFTISRDDSKNSLYLQMNSLKTEDTAVYYCAREGDYFDYWGQGTLVTVSS

SEQ ID NO: 257 - protein, 117 aa, synthetic construct
EVQLVESGGGLVQPGGSLRLSCAASGFTFSNYYLNWVRQAPGKGLEWVGDIRLKSNNYEKHYAESVKGRFTISRDDSKSITYLQMNSLRAEDTAVYYCAREGDYFDYWGQGTTVTSS

SEQ ID NO: 258 - protein, 117 aa, synthetic construct
EVQLVESGGGLVQPGGSLRLSCAASGFTFSFYYLNWVRQAPGKGLEWVGDIRLKKNNYEKHYAESVKGRFTISRDDSKSITYLQMNSLRAEDTAVYYCAREGDYFDYWGQGTTVTSS

SEQ ID NO: 259 - protein, 14 aa, synthetic construct
RSSQSVSSFGATFM

SEQ ID NO: 260 - protein, 7 aa, synthetic construct
LASRLGS

SEQ ID NO: 261 - protein, 9 aa, synthetic construct
QQNLQTPYT

SEQ ID NO: 262 - protein, 9 aa, synthetic construct
VQNNEDPYT

SEQ ID NO: 263 - protein, 111 aa, synthetic construct
DIVLTQSPASLAVSPGQRATITCKASESVDSFGATFMHWYQQKPGQPPKLLIYLASNRASGVPDRFSGSGSGTDFTLTINPVEADDTANYYCQQNNEDPYTFGQGTKLEIK

SEQ ID NO: 264 - protein, 111 aa, synthetic construct
EIVLTQSPGTLSLSPGERATLSCRASESVDSFGATFMHWYQQKPGQPPKLLIYLASNRASGVPDRFSGSGSGTDFTLKISRVEAEDVGVYYCQQNNEDPYTFGQGTKLEIK

SEQ ID NO: 265 - protein, 111 aa, synthetic construct
DIVLTQSPASLAVSPGQRATITCKASESVDSFGATFMHWYQQKPGQPPKLLIYLASNLESGVPARFSGSGSGTDFTLTINPVEADDTANYYCQQNLQTPYTFGQGTKLEIK

SEQ ID NO: 266 - protein, 111 aa, synthetic construct
DIVLTQSPASLAVSPGQRATITCKASESVDSFGATFMHWYLQKPGQSPQLLIYLASNLESGVPARFSGSGSGTDFTLKISRVEAEDVGVYYCQQNNEDPYTFGQGTKLEIK

SEQ ID NO: 267 - protein, 111 aa, synthetic construct
DIVLTQSPASLAVSPGQRATITCKASQSVSSFGATFMHWYQQKPGQPPKLLIYLASNLESGVPARFSGSGSGTDFTLKISRVEAEDVGVYYCQQNNEDPYTFGQGTKLEIK

SEQ ID NO: 268 - protein, 111 aa, synthetic construct
DIVLTQSPASLAVSPGQRATITCKASQSVSSFGATFMHWYLQKPGQSPQLLIYLASNLESGVPARFSGSGSGTDFTLTINPVEADDTANYYCQQNNEDPYTFGQGTKLEIK

SEQ ID NO: 269 - protein, 111 aa, synthetic construct
DIVMTQSPLSLPVTPGEPASISCRSSESVDSFGATFMHWYLQKPGQSPQLLIYLASNLESGVPARFSGSGSGTDFTLTINPVEADDTANYYCQQNNEDPYTFGQGTKLEIK

SEQ ID NO: 270 - protein, 111 aa, synthetic construct
DIVMTQSPLSLPVTPGEPASISCRSSESVDSFGATFMHWYLQKPGQSPQLLIYLASNLESGVPARFSGSGSGTDFTLTINPVEADDTANYYCQQNLQTPYTFGQGTKLEIK

SEQ ID NO: 271 - protein, 111 aa, synthetic construct
DIVMTQSPLSLPVTPGEPASISCRSSQSVSSFGATFMHWYQQKPGQPPKLLIYLASNLESGVPARFSGSGSGTDFTLTINPVEADDTANYYCQQNNEDPYTFGQGTKLEIK

SEQ ID NO: 272 - protein, 111 aa, synthetic construct
DIVMTQSPLSLPVTPGEPASISCRSSQSVSSFGATFMHWYQQKPGQPPKLLIYLASNRASGVPDRFSGSGSGTDFTLTINPVEADDTANYYCQQNNEDPYTFGQGTKLEIK

SEQ ID NO: 273 - protein, 111 aa, synthetic construct
EIVLTQSPGTLSLSPGERATLSCRASESVDSFGATFMHWYQQKPGQPPKLLIYLASNLESGVPARFSGSGSGTDFTLTINPVEADDTANYYCQQNLQTPYTFGQGTKLEIK

SEQ ID NO: 274 - protein, 111 aa, synthetic construct
EIVLTQSPGTLSLSPGERATLSCRASESVDSFGATFMHWYLQKPGQSPQLLIYLASNLESGVPARFSGSGSGTDFTLKISRVEAEDVGVYYCQQNNEDPYTFGQGTKLEIK

SEQ ID NO: 275 - protein, 111 aa, synthetic construct
EIVLTQSPGTLSLSPGERATLSCRASQSVSSFGATFMHWYQQKPGQPPKLLIYLASNRASGVPDRFSGSGSGTDFTLKISRVEAEDVGVYYCQQNLQTPYTFGQGTKLEIK

SEQ ID NO: 276 - protein, 111 aa, synthetic construct
EIVLTQSPGTLSLSPGERATLSCRASQSVSSFGATFMHWYLQKPGQSPQLLIYLASNLESGVPARFSGSGSGTDFTLTINPVEADDTANYYCQQNNEDPYTFGQGTKLEIK

SEQ ID NO: 277 - protein, 111 aa, synthetic construct
EIVLTQSPGTLSLSPGERATLSCRASQSVSSFGATFMHWYLQKPGQSPQLLIYLASNLESGVPARFSGSGSGTDFTLKISRVEAEDVGVYYCQQNNEDPYTFGQGTKLEIK

SEQ ID NO: 278 - protein, 111 aa, synthetic construct
DIQLTQSPSSLSASVGDRVTITCRASESVDSFGATFMHWYQQKPGKAPKLLIYLASNLESGVPSRFSGSGSGTDFTLTISSLQPEDFATYYCQQNNEDPYTFGQGTKLEIK

SEQ ID NO: 279 - protein, 111 aa, synthetic construct
DIQLTQSPSSLSASVGDRVTITCRASESVDSFGATFMHWYQQKPGKAPKLLIYLASRLGSGVPSRFSGSGSGTDFTLTISSLQPEDFATYYCVQNNEDPYTFGQGTKLEIK

SEQ ID NO: 280 - protein, 111 aa, synthetic construct
DIQLTQSPSSLSASVGDRVTITCRASESVDSFGATFMHWYQQKPGKAPKLLIYLASRLGSGVPSRFSGSGSGTDFTLTISSLQPEDFATYYCQQNNEDPYTFGQGTKLEIK

(19) United States
(12) Patent Application Publication
(10) Pub. No.: US 2025/0188189 A1
(43) Pub. Date: Jun. 12, 2025

(54) HUMAN ANTIBODIES AGAINST ACTIVATED PROTEIN C AND USES THEREOF

(71) Applicant: COAGULANT THERAPEUTICS CORPORATION, Dongdaemun-gu (KR)

(72) Inventors: Terry HERMISTON, Mill Valley, CA (US); Maxine BAUZON, Hercules, CA (US); Derek SIM, Burlingame, CA (US); Douglas M. SCHNEIDER, Layfayette, CA (US); Jan TEBBE, Köln (DE); Oliver KENSCH, Pulheim (DE); Ernst WEBER, Langenfeld (DE); Philipp ELLINGER, Düsseldorf (DE); Katharina FILARSKY, Düsseldorf (DE); Christoph MAHLERT, Wuppertal (DE); Bernardo MARTINS DA CUNHA ABECASIS, Lisboa (PT); Inês Margarida CASTRO FERREIRA, Paco de Arcos (PT)

(21) Appl. No.: 18/843,100

(22) PCT Filed: Mar. 3, 2023
(86) PCT No.: PCT/US2023/063716

§ 371 Date: Aug. 30, 2024

Related U.S. Application Data
(60) Provisional application No. 63/316,928, filed on Mar. 4, 2022.

(51) Int. Cl. C07K 16/40; A61P 7/04

(52) U.S. Cl. CPC C07K 16/40; A61P 7/04; C07K 2317/33; C07K 2317/565; C07K 2317/76; C07K 2317/92

(57) ABSTRACT

The present invention provides antibodies against activated protein C (APC). Certain disclosed antibodies inhibit the anticoagulant activity of APC while preserving its beneficial cytoprotective functions. The present invention also provides nucleic acids, vectors, and host cells for producing the antibodies disclosed herein, as well as methods of using the antibodies to treat medical conditions such as bleeding, sepsis, and inflammation.

### CROSS-REFERENCE TO RELATED APPLICATIONS

This application claims priority to U.S. Provisional Application No. 63/316,928 filed on Mar. 4, 2022, the contents of which are incorporated by reference in their entireties.

### SEQUENCE LISTING

This application includes a sequence listing in XML format titled “172085.00118_ST26.xml”, which is 412,954 bytes in size and was created on Feb. 28, 2023. The sequence listing is electronically submitted with this application via Patent Center and is incorporated herein by reference in its entirety.

### FIELD

This application concerns antibodies, antibody fragments, and other binders of activated protein C (APC) and related formulations, nucleic acids, methods of preparation, and methods of use.

### BACKGROUND

Protein C (PC) is a zymogen that is synthesized in the liver as a single-chain, 461-amino acid precursor (SEQ ID NO: 633) and secreted into the blood. Prior to secretion, the single-chain precursor is converted into a heterodimer by removal of a lysine-arginine dipeptide (amino acids 198-199 in SEQ ID NO: 633) and a 42-amino acid preproleader (amino acids 1-42 in SEQ ID NO: 633). The heterodimeric form (417 amino acids) consists of a light chain (155 amino acids, 21 kDa) and a heavy chain (262 amino acids, 41 kDa) linked by a disulfide bridge. The light chain contains one gamma-carboxy glutamic acid (Gla) domain (45 amino acids), two EGF-like domains (46 amino acids), and linker sequences, while the heavy chain contains a highly polar activation peptide (amino acids 200-211 in SEQ ID NO: 633) and a catalytic domain with a typical serine protease catalytic triad. Cleavage of the PC zymogen at the thrombin cleavage site leads to removal of the activation peptide and activation of PC to activated PC (APC; SEQ ID NO: 634; 405 amino acids).

Human PC undergoes extensive post-translational modifications, including glycosylation, vitamin K-dependent gamma-carboxylation, and gamma-hydroxylation. It contains four potential N-linked glycosylation sites: one in the light chain (Asn97) and three in the heavy chain (Asn248, Asn313, and Asn329). Its Gla domain contains nine Gla residues and is responsible for the calcium-dependent binding of PC to negatively charged phospholipid membranes. The Gla domain can also bind to endothelial protein C receptor (EPCR), which aligns thrombin and thrombomodulin on the endothelial membrane during PC activation.

PC activation occurs on the surface of endothelial cells in a two-step process. It requires binding of PC (via the Gla domain) to EPCR on endothelial cells, followed by proteolytic cleavage of PC by thrombin/thrombomodulin complexes at Arg12 of the heavy chain. This single cleavage liberates the 12-amino acid activation peptide, converting the zymogen PC into the active serine protease APC.

Under physiological conditions, APC circulates at very low concentrations (1-2 ng/ml or 40 pM) in human blood and has a half-life of 20-30 minutes, whereas PC circulates at a much higher concentration (3-5 μg/ml or 65 nM) and has a half-life of 6-8 hours.

The PC pathway plays a pivotal role in regulating coagulation and serves as a natural defense mechanism against thrombosis by amplifying an anticoagulant response as a coagulant response mounts. Upon injury, thrombin is generated to initiate coagulation. Thrombin also triggers an anticoagulant response by binding to thrombomodulin in the lining of the vascular surface, thereby promoting PC activation. As a result, APC generation is correlated with thrombin concentration and PC levels. APC functions as an anticoagulant by inactivating two coagulation cofactors (i.e., factor Va and factor VIIIa) via proteolytic cleavage, thereby inhibiting the generation of thrombin. APC also directly contributes to an enhanced fibrinolytic response via complex formation with plasminogen activator inhibitors.

Bleeding is a major problem, both in medical conditions and in connection with medical procedures. For example, bleeding causes complications following surgery, organ transplant, intracranial hemorrhage, aortic aneurysm, post-partum hemorrhage, trauma, and overdose of certain anticoagulants. In fact, the leading cause of death in humans 1-44 years of age is acute bleeding associated with traumatic injury. There are currently few options for treating life-threatening, non-compressible hemorrhage. Bleeding disorders may be treated by inhibiting APC to promote thrombin generation and coagulation. Thus, a first object of the present invention is to provide a safe and efficacious treatment for bleeding.

In addition to its anticoagulant functions, APC also induces cytoprotective effects via its anti-apoptotic, anti-inflammatory, and endothelial barrier stabilization activities, all of which contribute to the regenerative outcomes associated with APC (e.g., stimulation of neurogenesis, angiogenesis, and wound healing).

Ischemia is a life-threatening condition that results from an inadequate blood supply to an organ or tissue. An insufficient blood supply causes tissues to become starved of oxygen and can potentially result in a heart attack or stroke. In highly metabolically active tissues, such as the heart and brain, ischemia can cause irreversible damage in as little as 3-4 minutes. APC's cytoprotective effects include decreasing apoptosis and inhibiting expression of inflammatory mediators following ischemia. Thus, a second object of the present invention is to provide a safe and efficacious treatment for ischemia.

Studies of human diseases and animal models suggest that extracellular histones are involved in either the onset or development of inflammatory processes in various organs (Moiana et al., Clin Biochemistry 94 (2021) 12-19 at 15). Thus, histones are being studied as potential therapeutic targets in diseases in which inflammation and thrombosis play a key pathophysiological role. APC reduces the cytotoxicity of extracellular histones through histone proteolysis. This function of APC relies on a negatively charged exosite, which includes Glu330 and Glu333.

Extracellular histones are known to be major mediators of death in sepsis. Sepsis is a life-threatening condition that occurs when the body's response to infection causes injury to its own tissues and organs. Sepsis is a leading cause of morbidity and mortality in the United States, accounting for nearly 270,000 deaths annually. APC's cytoprotective effects include inhibiting histone toxicity associated with death in sepsis. Thus, a third object of the present invention is to provide a safe and efficacious treatment for sepsis.

Histones form part of neutrophil extracellular traps (NETs), networks of extracellular fibers, primarily composed of DNA from neutrophils. NETs have been shown to be involved in the pathogenesis and progression of cancer. Cancer is a leading cause of death worldwide, accounting for nearly 10 million deaths in 2020, or one in six deaths. NETs affect cancer via several mechanisms, including the establishment of an inflammatory microenvironment and interaction with other pro-tumor mechanisms such as inflammasomes and autophagy (Shao et al., Frontiers in Oncology vol. 11 article 714357 (2021) p. 1). Evidence suggests that NETs play a role in various cancer types, including breast, lung, colorectal, pancreatic, blood, neurological, and cutaneous cancers (Id. at p. 6). Thus, interventions that affect the PC pathway can potentially impact cancer, and a fourth object of the present invention is to provide a safe and efficacious treatment for cancer.

APC-based therapeutics have been under development for several years. Genetic engineering of APC has produced APC variants with a selective reduction of either its anticoagulant or cytoprotective activities. (Griffin et al., Blood 125:2898, 2015). For example, one such variant, 3K3A-APC (Lys191-193Ala), has less than 10% of wild-type APC's anticoagulant activity while retaining its full cytoprotective activity, and has advanced to clinical trials for ischemic stroke. However, previous attempts to translate research observations into APC-based medicines have failed, including the initial attempts to use APC as a treatment for sepsis. While encouraging clinical trial results led to the approval of a recombinant APC protein-based drug (drotrecogin alfa (activated), recombinant human activated protein C (rhAPC), trade name Xigris™) in 2001 (N Engl J Med 344:699, 2001), the positive initial findings were not replicated in subsequent randomized clinical trials, and this drug has been withdrawn from the market.

One potential problem with using exogenous proteins such as APC and APC variants as therapeutics is the potential for the recipient's immune system to generate anti-drug antibodies (ADAs) against them. The production of ADAs can have dramatic consequences, such as loss of efficacy and neutralization of the drug or other adverse events (J Pharm Sci 110 (7): 2575-2584, 2021). Thus, a fifth object of the present invention is to provide an APC-based therapeutic that has a lower immunogenicity risk.

Antibodies to APC have been explored as a means to selectively enhance or inhibit different functions of APC (see, e.g., US Pat. App. Pub. 20150307625 to Zhao et al. published Oct. 29, 2015; and US Pat. App. Pub. 2018326053 to Egner et al. published Nov. 15, 2018). For example, Magisetty et al. reported that a murine antibody that selectively inhibits the anticoagulant activity of APC while preserving its cytoprotective activity markedly reduced the severity of joint bleeding in a mouse model of hemophilic arthropathy, whereas an antibody that inhibits both the anticoagulant and cytoprotective activities of APC did not (Blood (2022) blood.2021013119). In Zhao et al., two different antibodies to APC were assessed as an APC-based treatment for hemophilia (Nat Commun 11 (1): 2992, 2020). One of the antibodies, termed class I, targeted the active site of APC, while the other, termed class II, targeted an exosite of APC. When these antibodies were administered to monkeys, both increased thrombin generation and promoted plasma clotting. However, whereas the class II antibody was well tolerated, the class I antibody resulted in animal death. Yet, the class II antibody still partially inhibited the active site of APC, which suggests that it may partially inhibit the cytoprotective effects of APC. Thus, a sixth object of the present invention is to provide an anti-APC antibody that maintains or enhances the cytoprotective effects of APC.

### SUMMARY

In a first aspect, the present invention provides isolated antibodies that specifically bind to activated protein C (APC) and minimally bind to unactivated protein C (PC). In some embodiments, the antibodies comprise a heavy chain variable region (V_{H}) comprising a CDR3 comprising an amino acid sequence selected from the group consisting of SEQ ID NOs: 9 and 118. In some embodiments, the antibodies comprise a V_{H} selected from the group consisting of NOs: 61-80 and 473-496. In some embodiments, the antibodies comprise a light chain variable region (V_{L}) comprising a CDR3 comprising an amino acid sequence selected from the group consisting of SEQ ID NOs: 23 and 138-140. In some embodiments, the antibodies comprise a V_{L} selected from the group consisting of SEQ ID NOs: 24-35 and 141-151. In some embodiments, the antibodies comprise both a V_{H} and a V_{L} described herein. In some embodiments, the antibodies comprise a heavy chain and a light chain that are both from a single antibody described herein.

In a second aspect, the present invention provides nucleic acids encoding the antibodies disclosed herein.

In a third aspect, the present invention provides vectors comprising the nucleic acids disclosed herein.

In a fourth aspect, the present invention provides host cells comprising a nucleic acid or vector disclosed herein.

In a fifth aspect, the present invention provides methods of producing an antibody disclosed herein. The methods comprise: (a) culturing a host cell disclosed herein under conditions that result in production of the antibody, and (b) isolating the antibody from the host cell.

In a sixth aspect, the present invention provides pharmaceutical compositions comprising an antibody disclosed herein and a pharmaceutically acceptable carrier.

In a seventh aspect, the present invention provides methods for treating or preventing a condition in a subject. The methods comprise administering a therapeutically effective amount of an antibody disclosed herein, an antibody disclosed herein specifically bound to an exogenous APC protein or variant thereof, or a pharmaceutical composition disclosed herein to the subject. In some embodiments, the condition is a condition that can be treated or prevented by enhancing or inhibiting the anticoagulant function of APC. In other embodiments, the condition is a condition that can be treated or prevented by enhancing or inhibiting the cytoprotective functions of APC.

In an eighth aspect, the present invention provides uses of the antibodies disclosed herein as a medicament.

### BRIEF DESCRIPTION OF THE DRAWINGS

FIG. 1 is a table presenting the results of the screening assays used to assess the 20 Set 1 antibodies. These results include protein expression levels in transiently transfected HEK293 cells, amidolytic activity (SPECTROZYME® PCa), binding to PC (i.e., K_{D}) based surface plasmon resonance (SPR), and aggregation temperature (Tagg) based on UNcle. The results are compared to those generated with the reformatted parental antibody TPP-24727 (highlighted in bold font).

FIG. 2 is a table presenting binding kinetics and affinity data for the Set 1 antibodies. These data were generated by performing SPR binding assays using human APC and PC as analytes and fitting the resulting sensorgrams to a 1:1 Langmuir binding model. The results are compared to those generated with the parental antibody TPP-24727 (highlighted in bold font).

FIG. 3 is a table presenting expression, purity, and stability data for four of the Set 1 antibodies. IgG concentrations were quantified via sandwich ELISA. Melting temperatures (Tm) were determined via differential scanning calorimetry using a MicroCal VP-Capillary DSC device, according to the manufacture's protocol.

FIG. 4 is a table presenting amidolytic activity data for human APC in the presence of the Set 1 antibodies at 8 nM and 3.2 nM. The results are compared to those generated with the parental antibody TPP-24727 (highlighted in bold font).

FIG. 5 is a set of three plots and tables presenting the results of a FXa- and thrombin-generation assay, which was used to measure the ability of the Set 1 antibodies to inhibit APC's inactivation of FVa. In this assay, FVa was incubated with each antibody at concentrations ranging from 0.5 to 1000 nM. After 30 minutes, a prothrombinase mixture containing FXa, prothrombin, and thrombin chromogenic substrate (S-2238) was added, and substrate hydrolysis was monitored using a plate reader.

FIG. 6A-FIG. 6E show the results of a Protac®-activated partial thromboplastin time (APTT) assay that was used to test the effects of the Set 1 antibodies on the clotting time of normal human plasma. This assay was performed in two independent screens (i.e., screen 1 and screen 2) on plasma samples containing different concentrations of the antibodies (i.e., 10 nM or 30 nM). The results are presented as a plot of clotting time at 30 nM versus clotting time at 10 nM in FIG. 6A and FIG. 6B and are summarized in a table in FIG. 6C. The results are also presented as a plot of rank order in screen 2 versus rank order in screen 1 in FIG. 6D and FIG. 6E.

FIG. 7 is a table showing secreted embryonic alkaline phosphatase (SEAP)-protease-activated receptor 1 (PAR1) cleavage data for human APC in the presence of the Set 1 antibodies. For this assay, SEAP-PAR1 was expressed on HEK293 cells with wild-type endothelial protein C receptor (wt-EPCR). APC was preincubated with the antibodies for 30 minutes before it was added to confluent SEAP-PAR1/wt-EPCR cells. After 60 minutes, SEAP release was measured using 1-step p-nitrophenyl phosphate.

FIG. 8A-FIG. 8B show the results of an endothelial barrier integrity assay for human APC in the presence of the Set 1 antibodies. The results are compared to those generated with the parental antibody TPP-24727. Endothelial cell permeability was measured in real time using the iCELLigence™ system. Confluent EA.hy926 endothelial cells were exposed to APC (50 nM) in the presence and absence of antibody. Permeability of confluent monolayers after treatment with APC for 3 hours was determined upon addition of thrombin (2 nM). The results are presented as a plot of percent barrier protection versus antibody concentration in FIG. 8A and are summarized in a table in FIG. 8B.

FIG. 9A-FIG. 9C show the results of a histone H3 cleavage assay for human APC in the presence of the Set 1 antibodies. A. APC (50 nM) and antibody (500 nM) were preincubated together for 30 minutes. After the incubation, the mixtures were added to 100 μg/ml histone H3. Over a period of 2 hours, the mixtures were subsampled at different timepoints to monitor the digestion of histone H3 over time. FIG. 9A shows the results generated with the parental antibody TPP-24727. FIG. 9B shows the results generated with each Set 1 antibody. FIG. 9C shows the results of a modified assay in which histone H3 digestion was measured in the presence of different ratios of antibody to APC after 1 hour of incubation.

FIG. 10 is a table showing the results of an in vitro plasma half-life assay for human APC in the presence of the Set 1 antibodies. In this assay, APC and antibody are preincubated for 30 minutes and normal pooled plasma is added. Samples are taken at different timepoints, and the residual chromogenic activity of APC is determined by one-phase exponential decay curve fitting.

FIG. 11 is a table outlining the mutations present in the Set 2 antibodies.

FIG. 12 is a table showing the results of the screening assays used to assess the Set 2 antibodies. These results include measures of antibody binding to APC and PC (i.e., K_{D}) based on SPR, the effect of the antibodies on the amidolytic activity (SPECTROZYME® PCa) of APC, and aggregation temperature (Tagg) based on UNcle. A hyphen indicates that the indicated data was not collected for the indicated antibody.

FIG. 13 shows the results of a Protac®-APTT assay that was used to test the effects of the Set 2 antibodies on the clotting time of normal human plasma. This assay was performed in two independent screens (i.e., screen 1 and screen 2) on plasma samples containing 30 nM of antibody. The results are presented as a plot of clotting time in screen 1 versus screen 2 (left) and are summarized in a table (right).

FIG. 14A-FIG. 14B show the results of a histone H3 cleavage assay for human APC in the presence of the Set 2 antibodies. APC (50 nM) and antibody (500 nM) were preincubated for 30 minutes. After the incubation, the mixtures were added to 100 μg/mL histone H3. Over a period of 2 hours, the mixtures were subsampled at different timepoints to monitor the digestion of the histone H3 over time. The digestion in the presence of each antibody was compared to the digestion in the presence of TPP-26870. The results are presented as a plot of histone H3 cleavage versus time in FIG. 14A and are summarized in a table in FIG. 14B.

FIG. 15 is a table showing the results of an in vitro plasma half-life assay for human APC in the presence of the Set 2 antibodies. In this assay, APC and antibody are preincubated for 30 minutes and normal pooled plasma is added. Samples are taken at different timepoints, and the residual chromogenic activity of APC is determined by one-phase exponential decay curve fitting.

FIG. 16 is a table in which the 20 Set 1 antibodies are divided into 7 functional categories based on their pharmacological properties, as determined in Example 3. In the anticoagulation activity column, a + indicates that the antibody is more inhibitory than the parental antibody TPP-24727, whereas a − indicates that the antibody is less inhibitory than TPP-24727. In the SEAP-PAR1 cleavage column, a + indicates that the antibody results in more PAR1 cleavage than TPP-24727, whereas a − indicates that the antibody results in less PAR1 cleavage than TPP-24727, and an ND indicates that no data was collected. In the histone cleavage column, a + indicates that the antibody results in more histone cleavage than TPP-24727, whereas a − indicates that the antibody results in less histone cleavage than TPP-24727, and an = indicates that the antibody results in similar histone cleavage to TPP-24727. In the sensitivity to physiological inhibitor column, a + indicates that the antibody makes APC more sensitive to physiological inhibitors than TPP-24727, whereas a − indicates that the antibody makes APC less sensitive to physiological inhibitors than TPP-24727, and an ND indicates that no data was collected.

FIG. 17 is a table in which the 24 Set 2 antibodies are divided into 11 functional categories based on their pharmacological properties, as determined in Example 4. In the anticoagulation activity column, a + indicates that the antibody is more inhibitory than the parental antibody TPP-26870, whereas a − indicates that the antibody is less inhibitory than TPP-26870. In the histone cleavage column, a + indicates that the antibody results in more histone cleavage than TPP-26870, whereas a − indicates that the antibody results in less histone cleavage than TPP-26870. In the sensitivity to physiological inhibitor column, a + indicates that the antibody makes APC more sensitive to physiological inhibitors than TPP-26870, whereas a − indicates that the antibody makes APC less sensitive to physiological inhibitors than TPP-26870, and an = indicates that the antibody results in similar APC sensitivity as TPP-26870.

FIG. 18 is a schematic outlining how the Set 1 and Set 2 antibodies were generated and screened.

FIG. 19 is a table showing the effect of humanizing the TPP-24727 heavy chain on immunogenicity score, expression level, and APC binding kinetics. The table is divided into a first group of antibodies that showed an overall improvement in these parameters, and a second group of antibodies that were deemed suboptimal based on this analysis. A third group of antibodies that was disclosed in International Patent Application Publication WO2023284012 is included for comparison (referred to in the figure as “OMRF”).

FIG. 20 is a table showing the effect of humanizing the TPP-24727 light chain on immunogenicity score, expression level, and APC binding kinetics. The table is divided into a first group of antibodies that showed an overall improvement in these parameters, and a second group of antibodies that were deemed suboptimal based on this analysis. A third group of antibodies that was disclosed in International Patent Application Publication WO2023284012 is included for comparison (referred to in the figure as “OMRF”). The immunogenicity scores of the improved antibodies were substantially lower than those of the antibodies disclosed in WO2023284012.

### DETAILED DESCRIPTION

The present disclosure relates to antibodies that specifically bind to activated protein C (APC). In preferred embodiments, the antibodies bind to the activated form of this enzyme (i.e., APC) and minimally bind to the zymogen form of this enzyme (i.e., protein C (PC)). In some embodiments, the disclosed antibodies inhibit the anticoagulant activity of APC while at least partially preserving or even enhancing the pleiotropic cytoprotective functions of APC. In some embodiments, the disclosed antibodies enhance the cytoprotective functions of APC while partially or completely preserving the anticoagulant activity of APC. The present invention also provides polynucleotides encoding these antibodies, pharmaceutical compositions comprising these antibodies, methods of making these antibodies, and methods for treating conditions by administering therapeutically effective amounts of these antibodies.

The anti-APC antibodies of the present invention offer several advantages over the recombinant APC protein therapeutics that are currently on the market or in development. For example, anti-APC antibodies have a lower risk of immunogenicity than therapeutics comprising exogenous APC proteins and variants thereof (e.g., 3K3A-APC). Anti-drug antibodies (ADAs) against exogenous APC proteins may cross-react with the endogenous PC/APC protein, resulting in an autoimmune response. For example, the APC variant 3K3A-APC from ZZ Biotech, LLC is in clinical trials where it is being administered to patients in a high number of repeated doses to treat ischemic stroke (ClinicalTrials.gov Trial Id. NCT02222714). This may effectively immunize patients, causing them to develop anti-APC antibodies that could cross-react with endogenous APC. In contrast, ADAs against anti-APC antibodies should not cross-react with endogenous PC/APC protein, making the use of anti-APC antibodies a safer therapeutic strategy. Further, depending on the selected antibody format, fewer doses of an anti-APC antibody may be required to achieve a desired therapeutic effect as compared to the number of doses required for an APC protein-based drug.

### Antibodies:

In a first aspect, the present invention provides antibodies that specifically bind to APC. As used herein, the term “antibody” refers to a protein that comprises at least one antigen-binding domain from an immunoglobulin. This term encompasses both full-length immunoglobulins and antigen-binding fragments thereof.

As stated above, the term “antibody” includes fragments of full-length immunoglobulins that comprise an antigen-binding domain. Examples of antigen-binding fragments include, without limitation, (i) Fab fragments, i.e., monovalent fragments consisting a heavy chain variable region (V_{H}), a light chain variable region (V_{L}), a constant domain of the κ light chain (CL), and a first constant domain of the heavy chain (CH1); (ii) F (ab′) 2 fragments, i.e., bivalent fragments comprising two Fab fragments linked by a disulfide bridge at the hinge region; (iii) Fd fragments consisting of the V_{H} and CH1; (iv) Fv fragments consisting of the V_{L} and V_{H} of a single arm of an antibody; (v) dAb fragments (Nature 341:544-546, 1989), which consists of a V_{H}; (vi) isolated complementarity determining regions (CDRs); (vii) minibodies, diabodies, triabodies, tetrabodies, and kappa bodies (see, e.g., Protein Eng 10:949-57, 1997); (viii) fragments of camelid antibodies, including V_{H}H antibodies, V_{H}H dimers, and V_{H}H-Fc fusions; and (ix) fragments of cartilaginous fish antibodies, including VNAR antibodies.

In some embodiments, the antibody is selected from the group consisting of an IgG1 antibody, an IgG2 antibody, an IgG3 antibody, an IgG4 antibody, an IgM antibody, an IgA1 antibody, an IgA2 antibody, a secretory IgA antibody, an IgD antibody, an IgE antibody, and antigen-binding fragments thereof. In other embodiments, the antibody comprises an alternative scaffold, such as a scaffold that comprises non-immunoglobulin binding proteins (e.g., an Affibody®, Affilin™, Affimer®, Alphabody, Anticalin®, Atrimer, Avimer, Centyrin, DARPin®, Fynomer, Kunitz domain, OBody, Pronectin®, or repebody). In certain preferred embodiments, the antibodies are in the IgG4 format. The IgG4 antibody format offers therapeutic advantages, as IgG4 is the only subclass of IgG that does not mediate common IgG effector functions, such as antibody-dependent cell-mediated cytotoxicity or complement dependent-cytotoxicity. Further, IgG4 antibodies are readily manufacturable and have been used in several commercially available therapeutics (e.g., natalizumab (Tysabri®), gemtuzumab (Mylotarg®), dupilumab (Dupixant®), nivolumab (Keytruda®)).

The antibodies of the present invention are anti-APC antibodies. As used herein, the term “anti-APC antibody” refers to an antibody that specifically binds to an epitope of APC. Preferably, the antibodies specifically bind to human APC, which has the amino acid sequence of SEQ ID NO: 242, or a variant thereof. As used herein, the term “specific binding” refers to an ability to bind to a particular antigen (e.g., APC) in preference to other molecules. Typically, an antibody that exhibits specific binding binds to an antigen with an equilibrium dissociation constant (K_{D}) of at least about 10⁻⁵ M and binds to that antigen with an affinity that is higher (e.g., at least two-fold higher) than its binding affinity for an irrelevant protein (e.g., BSA, casein). Often, a higher affinity (i.e., lower K_{D}, e.g., in the low nanomolar range) translates to a more potent and specific therapeutic, as it increases the likelihood that the antibody will locate and bind to its target antigen.

The antibodies disclosed herein have a high specificity for the activated form of protein C (APC), as opposed to the inactive zymogen form (PC). This is useful given that there is a 1700-fold difference in plasma concentrations of APC (˜40 pM) and PC (70 nM). Thus, it is preferable that the antibodies of the present invention minimally bind to PC. An antibody that “minimally binds” to a particular antigen either (a) does not bind to the antigen at detectable levels, or (b) binds to the antigen with an equilibrium dissociation constant (K_{D}) that is lower than about 10⁻² M.

The term “protein C” or “PC” may refer to any variant, isoform, or homolog of the zymogen PC. Preferably, PC is human PC, which has the amino acid sequence of SEQ ID NO: 241, or a variant thereof.

The term “epitope” refers to the region of an antigen to which an antibody specifically binds. For examples of APC epitopes, see U.S. Patent Application Publication 2018/326053, which describes epitopes outside of the catalytic triad of the active site of human APC. Conversely, the term “paratope” refers to the area of the antibody to which the antigen specifically binds.

Antibody binding activities may be assessed using methods that are known in the art, including enzyme-linked immunosorbent assay (ELISA), surface plasmon resonance (SPR), radioimmunoassay, bio-layer interferometry (BLI), and the like. For example, to ensure that the antibodies disclosed herein have a suitable association constant (i.e., kon) for use as human therapeutics, they were assessed via SPR analysis.

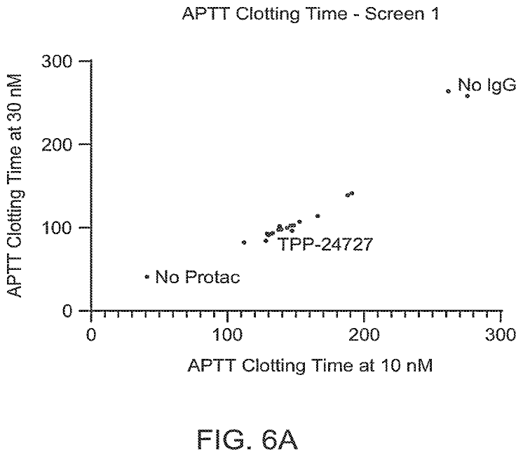

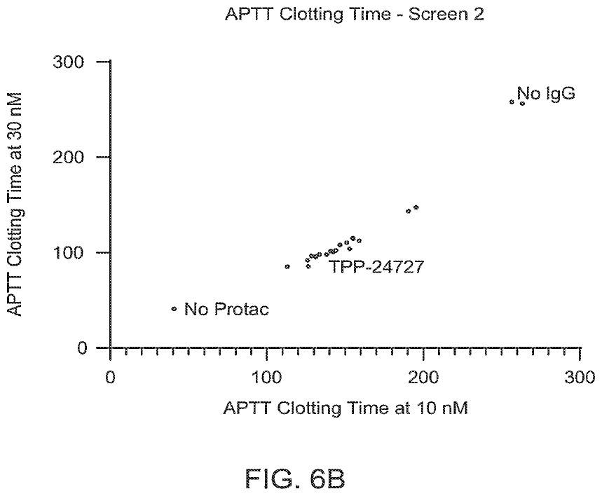

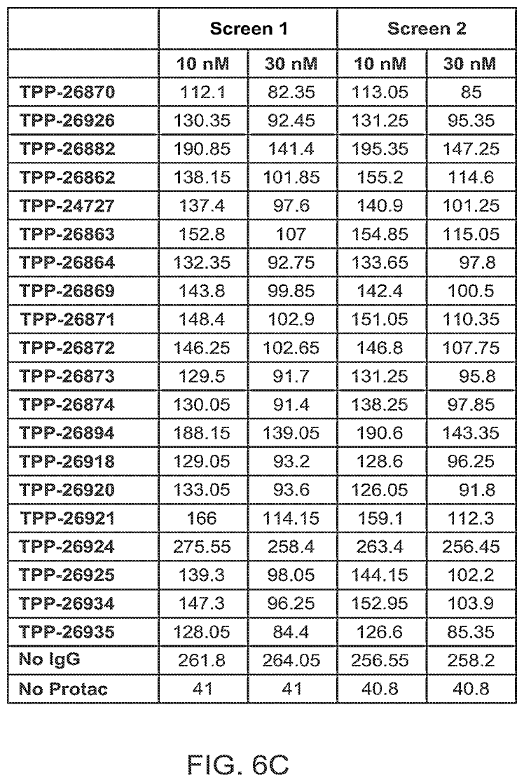

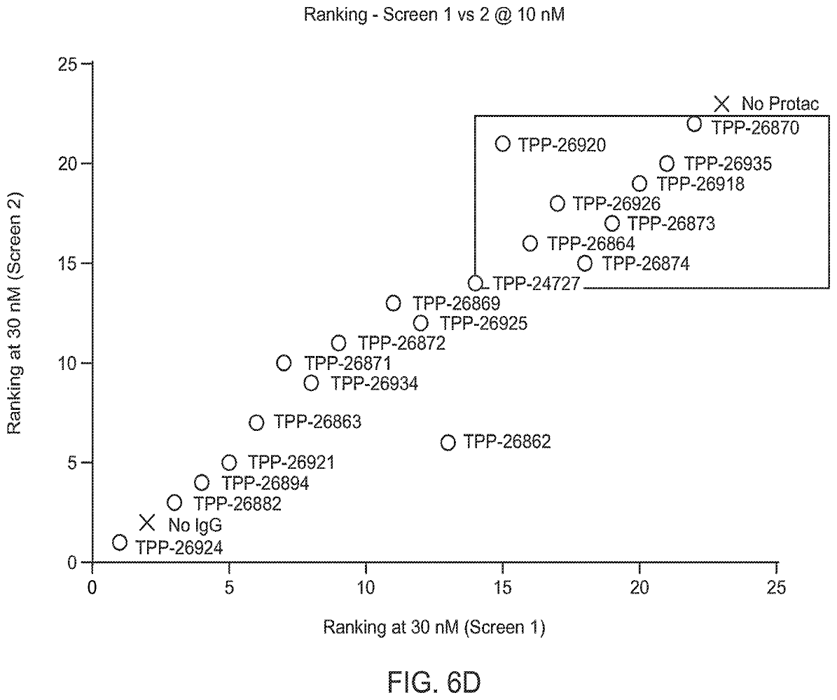

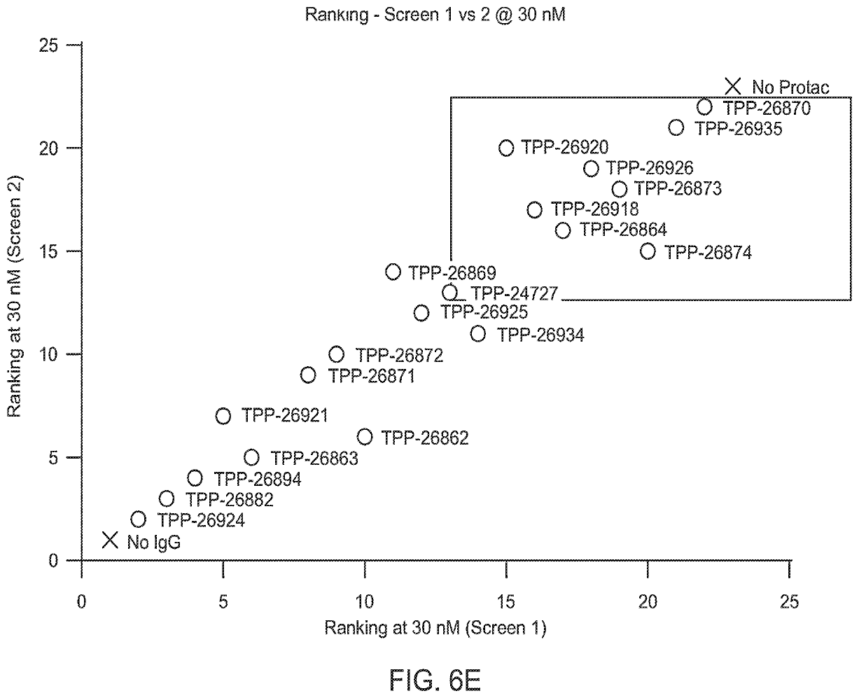

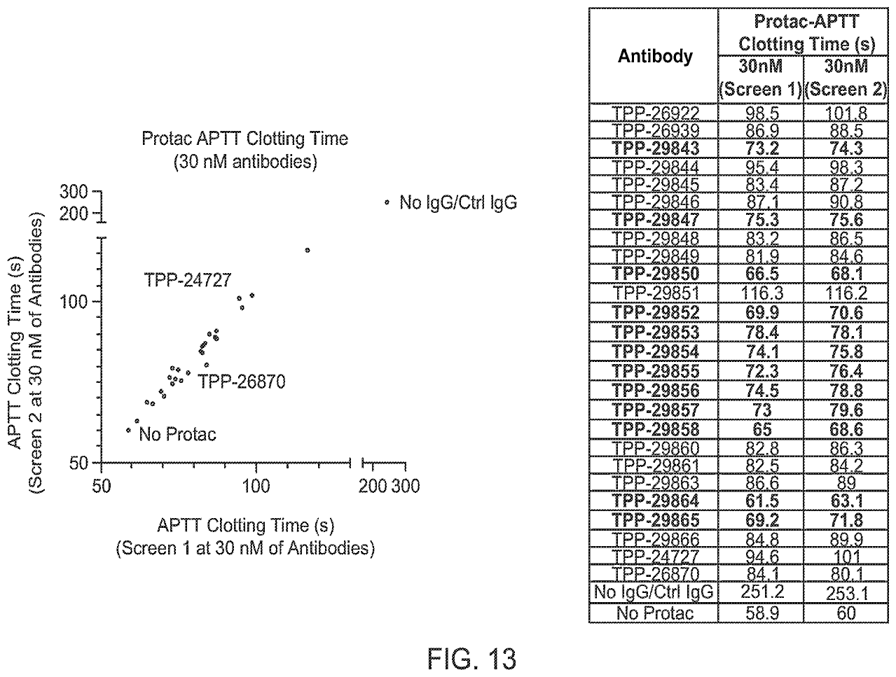

In some embodiments, the antibodies of the present invention inhibit the anticoagulant activity of APC (i.e., relative to the anticoagulant activity in a no-antibody control) and thereby promote blood clot formation. In some embodiments, the antibodies inhibit the anticoagulant activity of APC by at least about 10%, about 20%, about 30%, about 40%, about 50%, about 60%, about 70%, about 80%, about 90%, about 95%, about 96%, about 97%, about 98%, about 99%, or about 100%. The antibodies may exert this effect by blocking APC's ability to inactivate the clotting factors factor Va and factor VIIIa, thereby increasing thrombin generation. Suitable assays for assessing the ability of a particular antibody to inhibit the anticoagulant activity of APC include, without limitation, amidolytic activity assays, substrate cleavage assays (e.g., S-2366), and thrombin-generation assays. Alternatively, the ability of an antibody to decrease clotting time may be measured using an activated partial thromboplastin time (APTT) clotting assay. See Nat Commun 11 (1): 2992, 2020 for a description of these assays. In the Examples, the ability of anti-APC antibodies to inhibit the anticoagulant activity of APC was tested using a Protac®-modified APTT clotting assay (see FIG. 6 and FIG. 13).

For some indications, it may be preferable to use an anti-APC antibody that maintains or enhances the anticoagulant activity of APC. For example, such an antibody may be useful for treating patients with cancer-associated thrombosis, which is a major cause of mortality in cancer patients. Thus, in other embodiments, the antibodies of the present invention enhance or minimally affect the anticoagulant activity of APC.

As used herein, an antibody “minimally affects” a particular APC activity if the level of the APC activity in the presence of the antibody is within +/−20% of the level of the APC activity in a no-antibody control. In some cases, an antibody that “minimally affects” an APC activity is one that has no detectable effect on the APC activity (i.e., one for which the level of the APC activity in the presence of the antibody is the same as the level of the APC activity in a no-antibody control). As used herein, a “no-antibody control” is a comparable sample to which no anti-APC antibody has been added.

For most human therapeutics, it is preferable that an anti-APC antibody maintains or enhances APC's pleiotropic cytoprotective functions. The terms “cytoprotective functions” and “cytoprotective activities” are used interchangeably herein to describe the anti-apoptotic, anti-inflammatory, and endothelial barrier stabilization functions of APC, which all contribute to the regenerative outcomes associated with APC. These functions are often interrelated. For example, cell death (e.g., due to an injury or infection) leads to the release of histones into the extracellular space where they interact with endothelial cells, triggering endothelial cell apoptosis and contributing to systemic inflammation. Thus, the histone cleavage function of APC reduces apoptosis, endothelial barrier disruption, and inflammation.

However, for some indications, it may be preferable to use an anti-APC antibody that blocks APC's cytoprotective functions. For example, a short-term disruption in endothelial barrier function could be used to temporarily increase vascular permeability to induce inflammation for a therapeutic purpose. For cancer treatment, it may be advantageous to block cytoprotective activity to promote an immune response against a tumor.

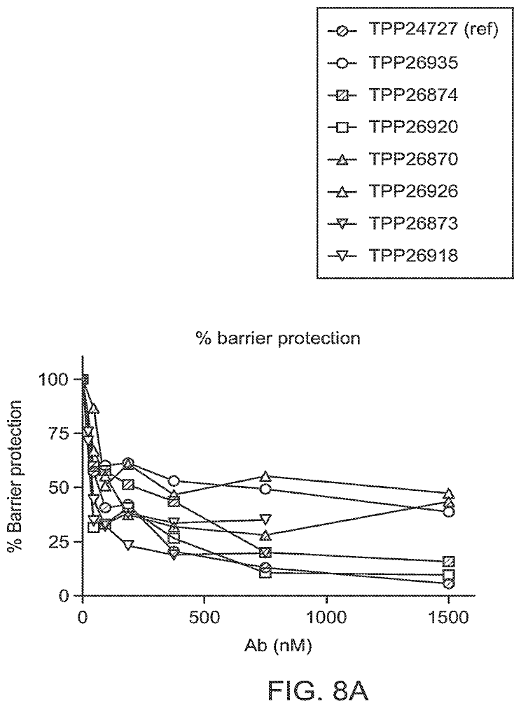

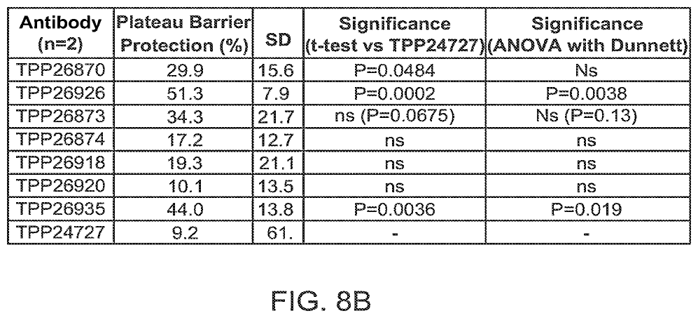

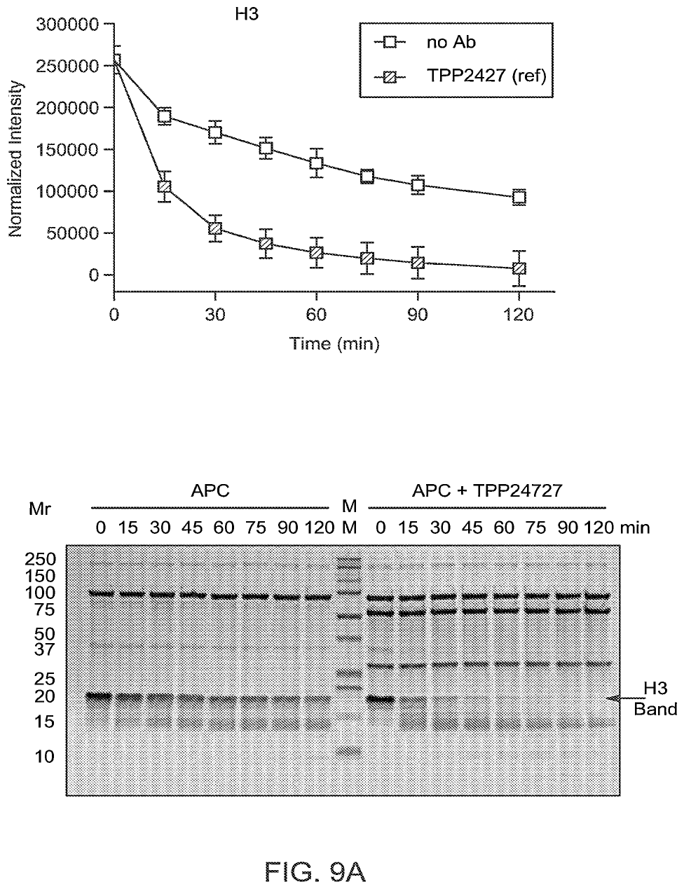

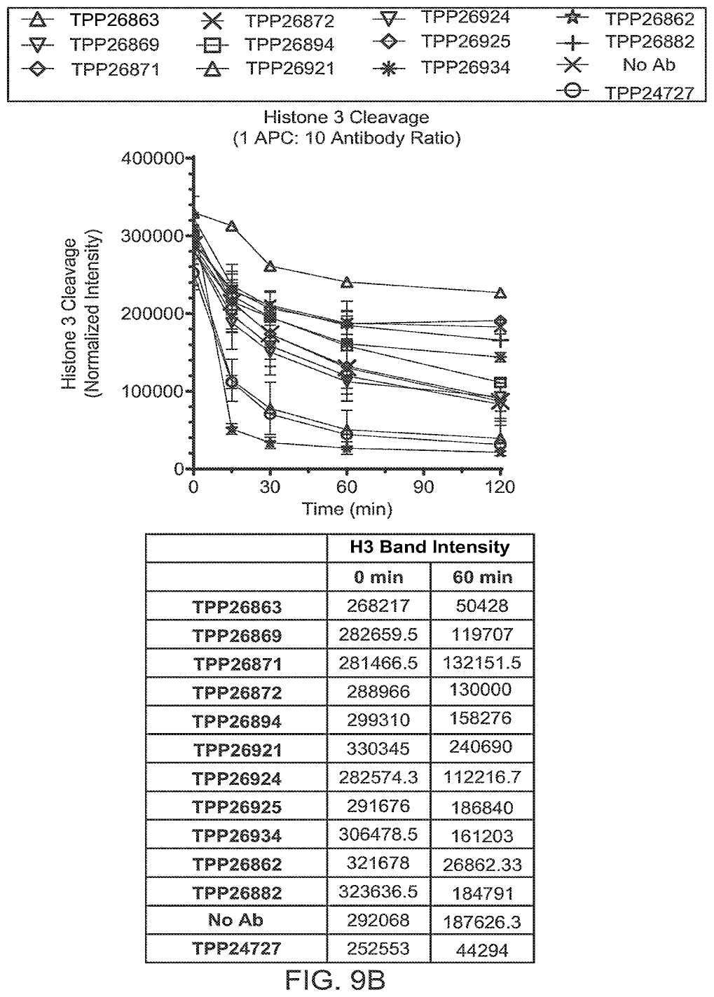

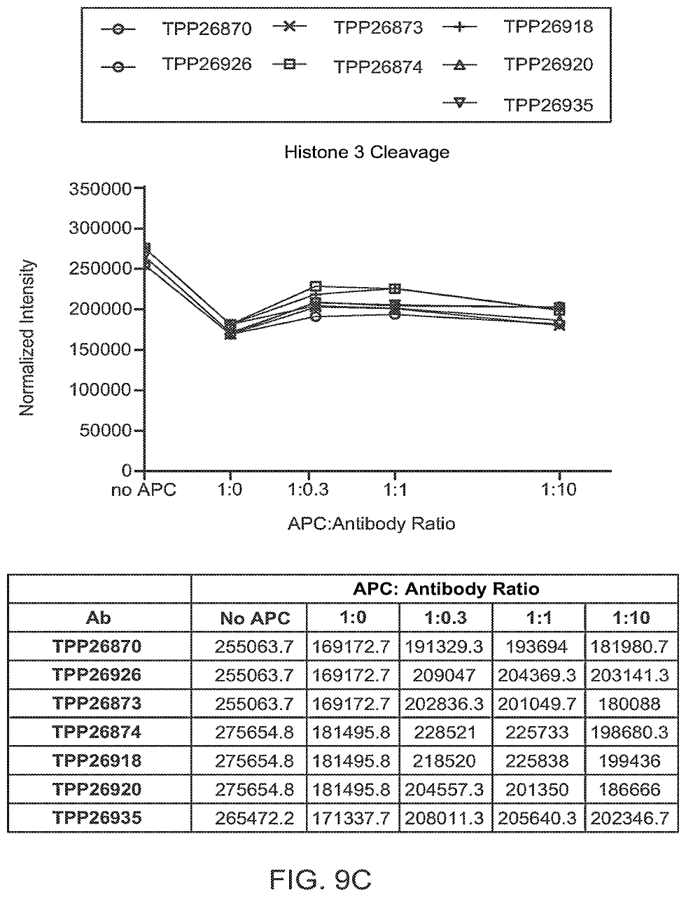

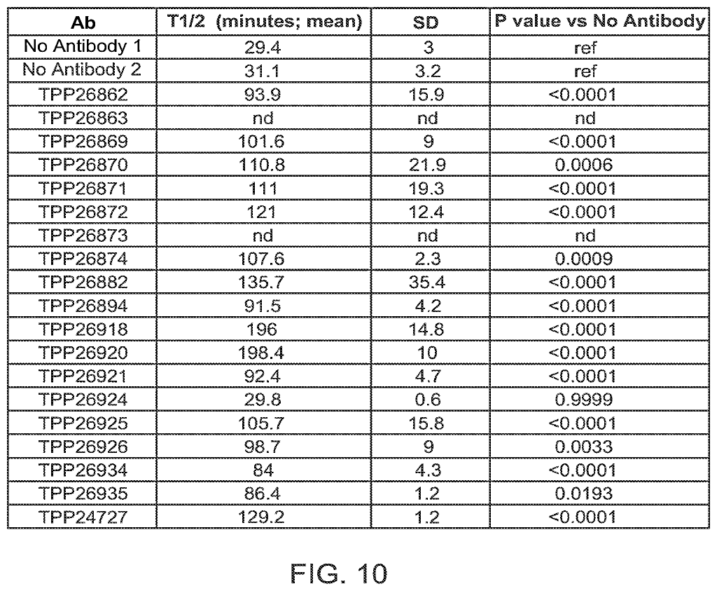

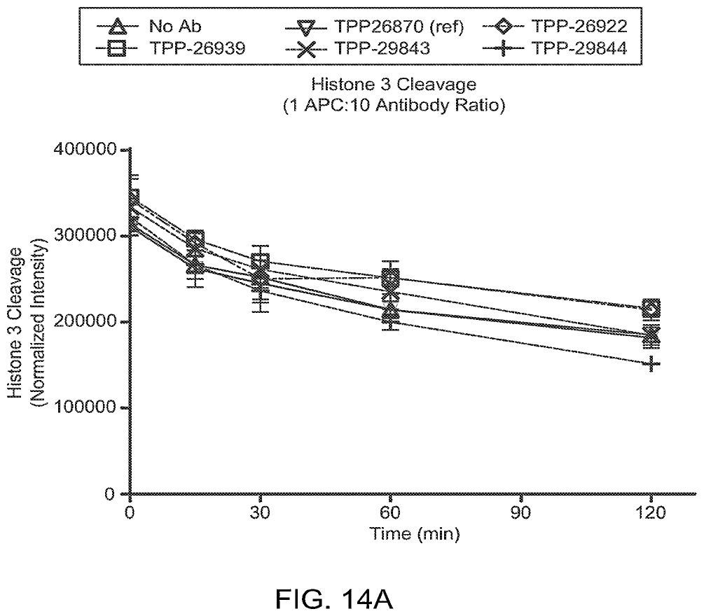

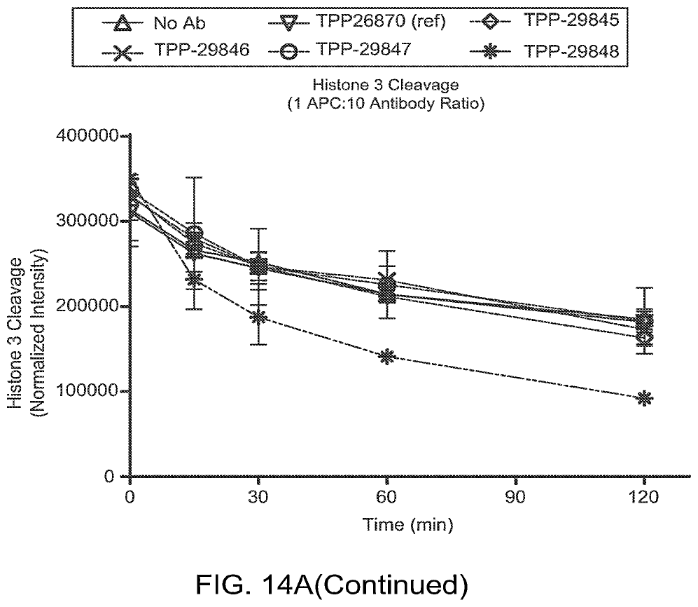

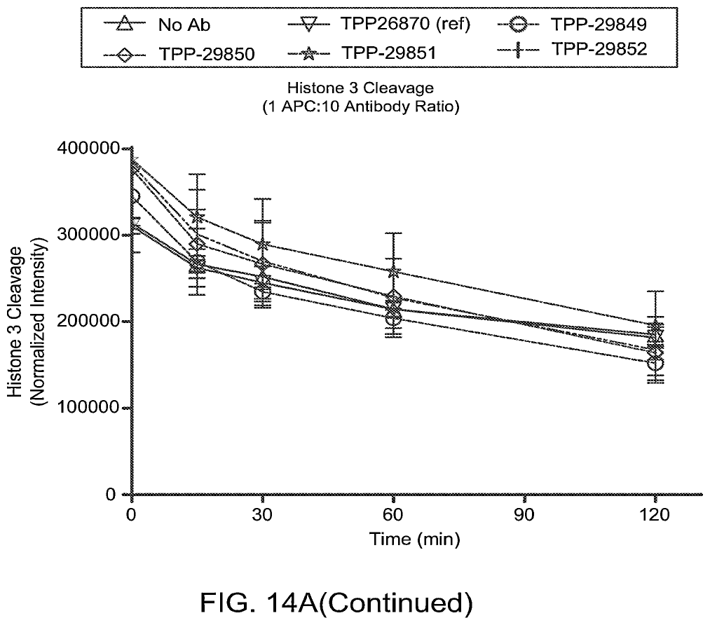

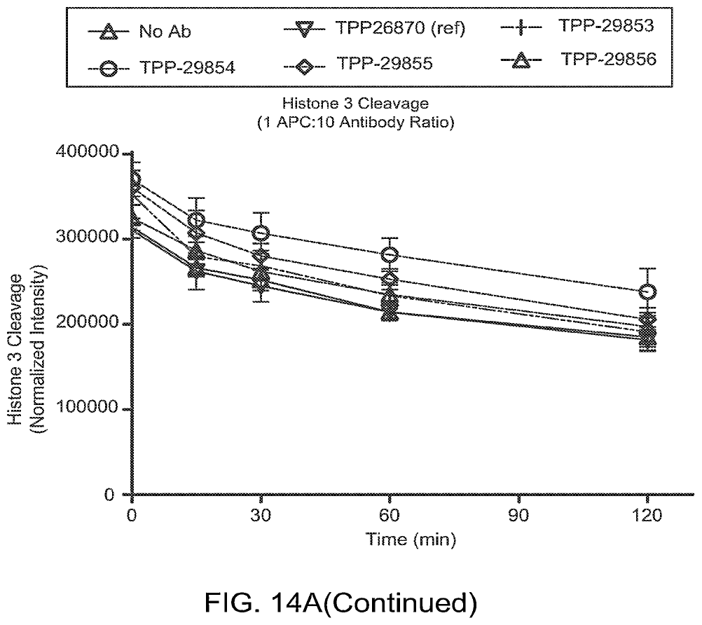

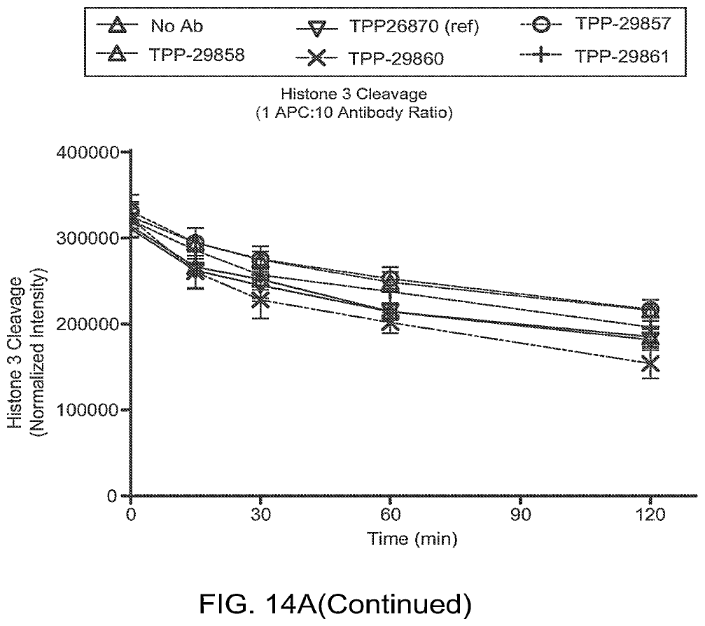

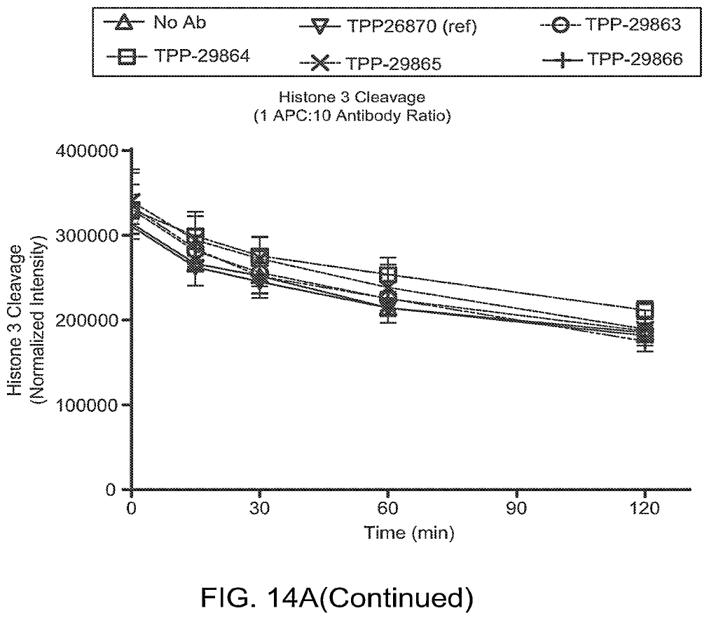

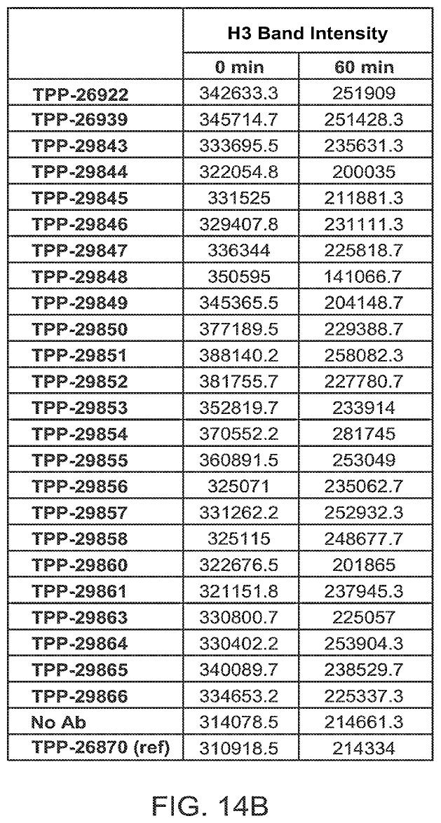

Thus, in some embodiments, the antibodies of the present invention enhance, minimally affect, or inhibit a cytoprotective function of APC (i.e., relative to the cytoprotective function in a no-antibody control). Specifically, in some embodiments, the antibodies (1) enhance, minimally affect, or inhibit APC-mediated histone cleavage, and/or (2) enhance, minimally affect, or inhibit affect APC-mediated endothelial barrier protection. In the Examples, the effects of anti-APC antibodies on APC-mediated histone cleavage were tested using a histone H3 cleavage assay (see FIG. 9 and FIG. 14) and the effects of anti-APC antibodies on APC-mediated endothelial barrier protection were tested using an in vitro endothelial barrier function assay (see FIG. 8).

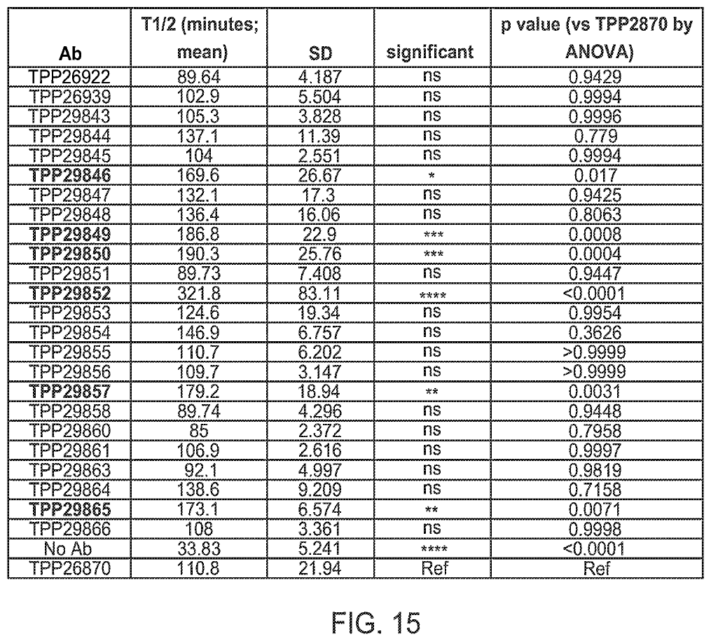

In some embodiments, the antibodies of the present invention increase or decrease the half-life of APC (i.e., relative to the half-life in the absence of the antibody). In other embodiments, the antibodies minimally affect the half-life of APC. In the Examples, the effects of anti-APC antibodies on the half-life of APC were tested using an in vitro plasma half-life assay (see FIG. 10 and FIG. 15).

The antibodies disclosed herein bind to exosites (i.e., sites other than the active site) of APC. The antibodies were generated from the exosite-binding parent antibody TPP-4885. TPP-4885 has been shown to inhibit APC's antithrombotic activity while persevering its beneficial cytoprotective functions (Nat Commun 11 (1): 2992, 2020). The separation of APC's anticoagulant and cytoprotective functions is possible because these functions involve distinct sites on the protein surface, i.e., the amino acid residues that mediate APC's interactions with cofactors and substrates are found in exosites that are far removed from the active site.

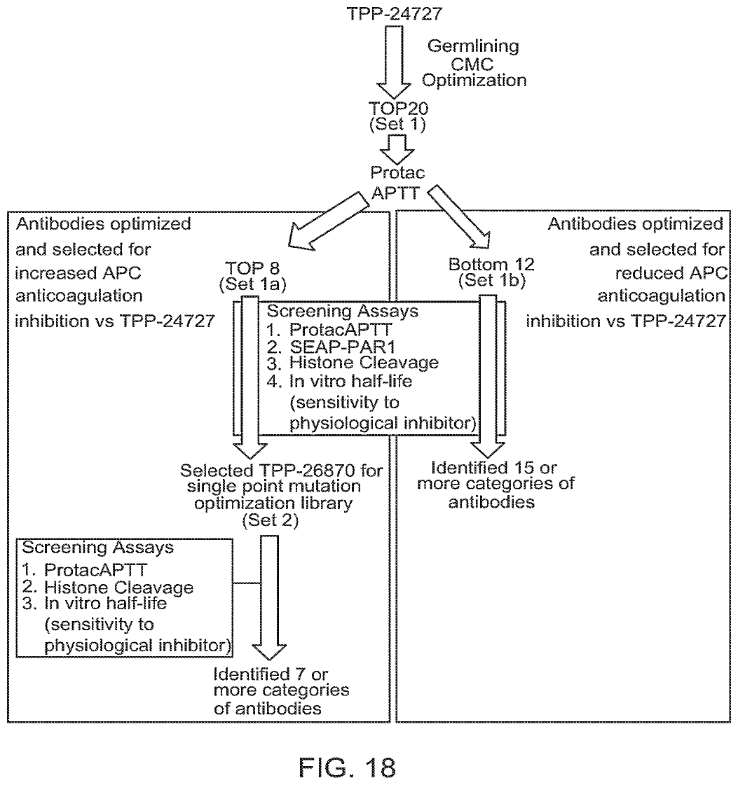

Example 1 describes the methods that were used to optimize the parental antibody TPP-4885. First, TPP-4885 (an IgG2 antibody) was reformatted into an IgG4 antibody and renamed TPP-24727. The IgG4 format that was used contains a serine 228 to proline (S228P) mutation that serves to prevent Fab-arm exchange. Next, stretches of the framework sequence from TPP-24727 were exchanged for human germline sequences to produce antibody frameworks that are more germline-like, generating 96 different antibody light chains and 96 different antibody heavy chains. The modified light chains and heavy chains were evaluated against the corresponding chain of the parent antibody (i.e., TPP-24727) using expression assays, enzymatic activity assays, and APC-binding assays. Based on these assays, 13 light chains and 6 heavy chains were selected and combined to generate 78 optimized antibodies. The 78 new antibodies were re-screened using the same assays, and 20 antibodies were selected for further study. These 20 antibodies are referred to in the present application as the “Set 1 antibodies.” The generation and characterization of the Set 1 antibodies is described in greater detail in Examples 1-3. The sequence identifiers for the amino acid and DNA sequences of the Set 1 antibodies are listed in Table 1 and Table 2, respectively. A second set of 24 antibodies, referred to herein as the “Set 2 antibodies,” was generated and evaluated as described in Example 4. The Set 2 antibodies include 22 antibody variants of the promising Set 1 antibody TPP-26870 that were generated via random mutagenesis. The Set 2 antibodies also include two additional antibodies (i.e., TPP-26922 and TPP-26939) that are not related to any of the Set 1 antibodies. The sequence identifiers for the amino acid and DNA sequences of the Set 2 antibodies are listed in Table 3 and Table 4, respectively. A schematic outlining how the Set 1 and Set 2 antibodies were generated and screened is provided in FIG. 18.

TABLE 1
Sequence identifiers for the amino acid sequences of the CDRs, V_{H}, V_{L}, full-length
heavy chain, and full-length light chain of the Set 1 antibodies disclosed herein

| | Amino acid sequences | | | | | | | | | |
|---|---|---|---|---|---|---|---|---|---|---|
| | V_{H} | | | | V_{L} | | | | | |
| | | | | Full- | | | | Full- | Heavy | Light |
| Antibody name | CDR1 | CDR2 | CDR3 | length | CDR1 | CDR2 | CDR3 | length | chain | chain |
| TPP-24727 | 1 | 5 | 9 | 10 | 16 | 21 | 23 | 24 | 36 | 42 |
| TPP-26862 | 2 | 6 | 9 | 11 | 17 | 22 | 23 | 25 | 37 | 43 |
| TPP-26863 | 2 | 6 | 9 | 11 | 17 | 22 | 23 | 26 | 37 | 44 |
| TPP-26864 | 2 | 6 | 9 | 11 | 18 | 22 | 23 | 27 | 37 | 45 |
| TPP-26869 | 2 | 6 | 9 | 11 | 19 | 22 | 23 | 28 | 37 | 46 |
| TPP-26870 | 2 | 6 | 9 | 11 | 19 | 22 | 23 | 29 | 37 | 47 |
| TPP-26871 | 2 | 6 | 9 | 11 | 20 | 22 | 23 | 30 | 37 | 48 |
| TPP-26872 | 2 | 6 | 9 | 11 | 20 | 22 | 23 | 31 | 37 | 49 |
| TPP-26873 | 2 | 6 | 9 | 11 | 20 | 22 | 23 | 32 | 37 | 50 |
| TPP-26874 | 2 | 6 | 9 | 11 | 20 | 22 | 23 | 33 | 37 | 51 |
| TPP-26882 | 2 | 7 | 9 | 12 | 19 | 22 | 23 | 28 | 38 | 46 |
| TPP-26894 | 2 | 8 | 9 | 13 | 19 | 22 | 23 | 34 | 39 | 52 |
| TPP-26918 | 3 | 5 | 9 | 14 | 16 | 22 | 23 | 35 | 40 | 53 |
| TPP-26920 | 3 | 5 | 9 | 14 | 19 | 22 | 23 | 34 | 40 | 52 |
| TPP-26921 | 3 | 5 | 9 | 14 | 19 | 22 | 23 | 28 | 40 | 46 |
| TPP-26924 | 3 | 5 | 9 | 14 | 20 | 22 | 23 | 31 | 40 | 49 |
| TPP-26925 | 3 | 5 | 9 | 14 | 20 | 22 | 23 | 32 | 40 | 50 |
| TPP-26926 | 3 | 5 | 9 | 14 | 20 | 22 | 23 | 33 | 40 | 51 |
| TPP-26934 | 4 | 5 | 9 | 15 | 19 | 22 | 23 | 28 | 41 | 46 |
| TPP-26935 | 4 | 5 | 9 | 15 | 19 | 22 | 23 | 29 | 41 | 47 |

TABLE 2
Sequence identifiers for the DNA sequences of the CDRs, V_{H}, V_{L}, full-length heavy
chain, and full-length light chain of the Set 1 antibodies disclosed herein

| | DNA sequences | | | | | | | | | |
|---|---|---|---|---|---|---|---|---|---|---|
| | V_{H} | | | | V_{L} | | | | | |
| Antibody | | | | Full- | | | | Full- | Heavy | Light |
| name | CDR1 | CDR2 | CDR3 | length | CDR1 | CDR2 | CDR3 | length | chain | chain |
| TPP-24727 | 54 | 58 | 62 | 63 | 69 | 74 | 78 | 79 | 91 | 97 |
| TPP-26862 | 55 | 59 | 62 | 64 | 70 | 75 | 78 | 80 | 92 | 98 |
| TPP-26863 | 55 | 59 | 62 | 64 | 70 | 76 | 78 | 81 | 92 | 99 |
| TPP-26864 | 55 | 59 | 62 | 64 | 71 | 77 | 78 | 82 | 92 | 100 |
| TPP-26869 | 55 | 59 | 62 | 64 | 72 | 75 | 78 | 83 | 92 | 101 |
| TPP-26870 | 55 | 59 | 62 | 64 | 72 | 75 | 78 | 84 | 92 | 102 |
| TPP-26871 | 55 | 59 | 62 | 64 | 73 | 75 | 78 | 85 | 92 | 103 |
| TPP-26872 | 55 | 59 | 62 | 64 | 73 | 76 | 78 | 86 | 92 | 104 |
| TPP-26873 | 55 | 59 | 62 | 64 | 73 | 76 | 78 | 87 | 92 | 105 |
| TPP-26874 | 55 | 59 | 62 | 64 | 73 | 75 | 78 | 88 | 92 | 106 |
| TPP-26882 | 55 | 60 | 62 | 65 | 72 | 75 | 78 | 83 | 93 | 101 |
| TPP-26894 | 55 | 61 | 62 | 66 | 72 | 76 | 78 | 89 | 94 | 107 |
| TPP-26918 | 56 | 58 | 62 | 67 | 69 | 75 | 78 | 90 | 95 | 108 |
| TPP-26920 | 56 | 58 | 62 | 67 | 72 | 76 | 78 | 89 | 95 | 107 |
| TPP-26921 | 56 | 58 | 62 | 67 | 72 | 75 | 78 | 83 | 40 | 101 |
| TPP-26924 | 56 | 58 | 62 | 67 | 73 | 76 | 78 | 86 | 95 | 104 |
| TPP-26925 | 56 | 58 | 62 | 67 | 73 | 76 | 78 | 87 | 95 | 105 |
| TPP-26926 | 56 | 58 | 62 | 67 | 73 | 75 | 78 | 88 | 95 | 106 |
| TPP-26934 | 57 | 58 | 62 | 68 | 72 | 75 | 78 | 83 | 96 | 101 |
| TPP-26935 | 57 | 58 | 62 | 68 | 72 | 75 | 78 | 84 | 96 | 102 |

TABLE 3
Sequence identifiers for the amino acid sequences of the CDRs, V_{H}, V_{L}, full-length
heavy chain, and full-length light chain of the Set 2 antibodies disclosed herein

| | Amino acid sequences | | | | | | | | | |
|---|---|---|---|---|---|---|---|---|---|---|
| | V_{H} | | | | V_{L} | | | | | |
| Antibody | | | | Full- | | | | Full- | Heavy | Light |
| name | CDR1 | CDR2 | CDR3 | length | CDR1 | CDR2 | CDR3 | length | chain | chain |
| TPP-26922 | 3 | 5 | 9 | 14 | 19 | 22 | 23 | 29 | 40 | 47 |
| TPP-26939 | 4 | 5 | 9 | 15 | 20 | 22 | 23 | 33 | 41 | 51 |
| TPP-29843 | 2 | 114 | 9 | 119 | 19 | 22 | 23 | 29 | 152 | 47 |
| TPP-29844 | 109 | 6 | 9 | 120 | 19 | 22 | 23 | 29 | 153 | 47 |
| TPP-29845 | 110 | 6 | 9 | 121 | 19 | 22 | 23 | 29 | 154 | 47 |
| TPP-29846 | 111 | 6 | 9 | 122 | 19 | 22 | 23 | 29 | 155 | 47 |
| TPP-29847 | 2 | 6 | 9 | 11 | 19 | 22 | 138 | 141 | 37 | 163 |
| TPP-29848 | 2 | 6 | 9 | 11 | 130 | 22 | 23 | 142 | 37 | 164 |
| TPP-29849 | 2 | 6 | 9 | 11 | 19 | 132 | 23 | 143 | 37 | 165 |
| TPP-29850 | 2 | 6 | 9 | 11 | 19 | 133 | 23 | 144 | 37 | 166 |
| TPP-29851 | 3 | 6 | 9 | 123 | 19 | 22 | 23 | 29 | 156 | 47 |
| TPP-29852 | 112 | 6 | 9 | 124 | 19 | 22 | 23 | 29 | 157 | 47 |
| TPP-29853 | 2 | 6 | 118 | 125 | 19 | 22 | 23 | 29 | 158 | 47 |
| TPP-29854 | 2 | 6 | 9 | 11 | 19 | 134 | 23 | 145 | 37 | 167 |
| TPP-29855 | 2 | 115 | 9 | 126 | 19 | 22 | 23 | 29 | 159 | 47 |
| TPP-29856 | 2 | 6 | 9 | 11 | 19 | 135 | 23 | 146 | 37 | 168 |
| TPP-29857 | 2 | 6 | 9 | 11 | 19 | 22 | 139 | 147 | 37 | 169 |
| TPP-29858 | 2 | 6 | 9 | 11 | 19 | 136 | 23 | 148 | 37 | 170 |
| TPP-29860 | 2 | 6 | 9 | 11 | 19 | 22 | 140 | 149 | 37 | 171 |
| TPP-29861 | 2 | 116 | 9 | 127 | 19 | 22 | 23 | 29 | 160 | 47 |
| TPP-29863 | 2 | 6 | 9 | 11 | 131 | 22 | 23 | 150 | 37 | 172 |
| TPP-29864 | 2 | 6 | 9 | 11 | 19 | 137 | 23 | 151 | 37 | 173 |
| TPP-29865 | 113 | 6 | 9 | 128 | 19 | 22 | 23 | 29 | 161 | 47 |
| TPP-29866 | 2 | 117 | 9 | 129 | 19 | 22 | 23 | 29 | 162 | 47 |

TABLE 4
Sequence identifiers for the DNA sequences of the CDRs, V_{H}, V_{L}, full-length heavy
chain, and full-length light chain of the Set 2 antibodies disclosed herein

| | DNA sequences | | | | | | | | | |
|---|---|---|---|---|---|---|---|---|---|---|
| | V_{H} | | | | V_{L} | | | | | |
| Antibody | | | | Full- | | | | Full- | Heavy | Light |
| name | CDR1 | CDR2 | CDR3 | length | CDR1 | CDR2 | CDR3 | length | chain | chain |
| TPP-26922 | 56 | 58 | 62 | 67 | 72 | 75 | 78 | 84 | 95 | 102 |
| TPP-26939 | 57 | 58 | 62 | 68 | 73 | 75 | 78 | 88 | 96 | 106 |
| TPP-29843 | 55 | 179 | 62 | 185 | 72 | 75 | 78 | 84 | 219 | 102 |
| TPP-29844 | 174 | 180 | 62 | 186 | 72 | 75 | 78 | 84 | 220 | 102 |
| TPP-29845 | 175 | 59 | 62 | 187 | 72 | 75 | 78 | 84 | 221 | 102 |
| TPP-29846 | 176 | 59 | 62 | 188 | 72 | 75 | 78 | 84 | 222 | 102 |
| TPP-29847 | 55 | 59 | 62 | 64 | 72 | 75 | 205 | 208 | 92 | 230 |
| TPP-29848 | 55 | 59 | 62 | 64 | 196 | 198 | 78 | 209 | 92 | 231 |
| TPP-29849 | 55 | 59 | 62 | 64 | 72 | 199 | 78 | 210 | 92 | 232 |
| TPP-29850 | 55 | 59 | 62 | 64 | 72 | 200 | 78 | 211 | 92 | 233 |
| TPP-29851 | 56 | 59 | 62 | 189 | 72 | 75 | 78 | 84 | 223 | 102 |
| TPP-29852 | 177 | 59 | 62 | 190 | 72 | 75 | 78 | 84 | 224 | 102 |
| TPP-29853 | 55 | 59 | 184 | 191 | 72 | 75 | 78 | 84 | 225 | 102 |
| TPP-29854 | 55 | 59 | 62 | 64 | 72 | 201 | 78 | 212 | 92 | 234 |
| TPP-29855 | 55 | 181 | 62 | 192 | 72 | 75 | 78 | 84 | 226 | 102 |
| TPP-29856 | 55 | 59 | 62 | 64 | 72 | 202 | 78 | 213 | 92 | 235 |
| TPP-29857 | 55 | 59 | 62 | 64 | 72 | 75 | 206 | 214 | 92 | 236 |
| TPP-29858 | 55 | 59 | 62 | 64 | 72 | 203 | 78 | 215 | 92 | 237 |
| TPP-29860 | 55 | 59 | 62 | 64 | 72 | 75 | 207 | 216 | 92 | 238 |
| TPP-29861 | 55 | 182 | 62 | 193 | 72 | 75 | 78 | 84 | 227 | 102 |
| TPP-29863 | 55 | 59 | 62 | 64 | 197 | 75 | 78 | 217 | 92 | 239 |
| TPP-29864 | 55 | 59 | 62 | 64 | 72 | 204 | 78 | 218 | 92 | 240 |
| TPP-29865 | 178 | 59 | 62 | 194 | 72 | 75 | 78 | 84 | 228 | 102 |
| TPP-29866 | 55 | 183 | 62 | 195 | 72 | 75 | 78 | 84 | 229 | 102 |

The antibodies of the present invention are “isolated,” meaning that they are substantially free of other biological molecules, including antibodies having different antigenic specificities and other cellular materials. In some embodiments, the isolated antibodies are at least about 75%, about 80%, about 90%, about 95%, about 97%, about 99%, about 99.9% or about 100% pure by dry weight. Purity may be measured using standard method such as column chromatography, polyacrylamide gel electrophoresis, or high-performance liquid chromatography (HPLC). An isolated antibody that binds to human APC can, however, have cross-reactivity to other closely related antigens, e.g., APC homologs from other species.

The classical representation of an antibody is as a Y-shaped molecule composed of four polypeptide subunits. Each antibody comprises two identical copies of a longer heavy chain, and two identical copies of a shorter light chain. The light chains of an antibody can be classified as either kappa (κ) or lambda (λ) type based on small differences in polypeptide sequence. The heavy chain makeup determines the overall class of each antibody. Each heavy chain has two regions, the heavy chain constant region and the heavy chain variable region (V_{H}). Likewise, each light chain comprises a light chain constant region and a light chain variable region (V_{L}). The constant regions are identical in all antibodies of the same isotype, but differ between antibodies of different isotypes, whereas the variable regions are antibody specific and differ depending on the B cell that produced it. Together, the two V_{H} and the two V_{L} form the variable region of the antibody, which serves as the antigen-binding site.

Each V_{H} and V_{L} of an antibody comprises three complementarity-determining regions. The term “complementarity-determining regions” or “CDRs” refers to hypervariable regions that together form an antigen-binding surface that is complementary to the three-dimensional structure of the antigen. The CDRs are numbered as “CDR1,” “CDR2,” and “CDR3 starting from the N-terminus of the V_{H} or V_{L} (see Proc Natl Acad Sci USA 72 (12): 5107, 1975; J Exp Med 132 (2): 211, 1970). In conventional antibodies (i.e., antibodies that comprise two heavy chains and two light chains), an antigen-binding site includes six CDRs: the three CDRs of the V_{H} and the three CDRs of the V_{L}.

Because CDR sequences are responsible for most antibody-antigen interactions, it is possible to create recombinant antibodies that mimic the properties of a specific naturally occurring antibody by grafting the CDR sequences of the naturally occurring antibody into the framework sequences of a different antibody with different properties (see, e.g., Nature 332:323-327,1998; Nature 321:522-525, 1986; Proc Natl Acad Sci USA 86:10029-10033, 1989). Such framework sequences can be obtained from public databases that include germline antibody gene sequences. Thus, in some embodiments, the CDRs of the antibodies described herein are grafted into another antibody framework.

Of the three CDRs, CDR3 is believed to be the main contributor for antigen recognition and specificity, and CDR1 and CDR2 are believed to contribute to binding strength (J Mol Biol 430:4369, 2018; Protein Eng Des Sel 31:267, 2018; Proteins 86:697, 2018). Thus, in some embodiments the antibodies of the present invention comprise a CDR3 of an antibody disclosed herein. Specifically, in some embodiments, the antibodies of the present invention comprise a V_{H} comprising a CDR3 comprising or consisting of an amino acid sequence selected from the group consisting of SEQ ID NOs: 9 and 118. Further, in some embodiments, the antibodies comprise a V_{L} comprising a CDR3 comprising or consisting of an amino acid sequence selected from the group consisting of SEQ ID NOs: 23 and 138-140.

In some embodiments, the antibodies of the present invention further comprise a CDR1 and a CDR2 of an antibody disclosed herein. Specifically, in some embodiments, the V_{H} of the antibody may comprise (a) a CDR1 comprising or consisting of an amino acid sequence selected from the group consisting of SEQ ID NOs: 1-4 and 109-113, (b) a CDR2 comprising or consisting of an amino acid sequence selected from the group consisting of SEQ ID NOs: 5-8 and 114-117, or (c) both a CDR1 comprising or consisting of an amino acid sequence selected from the group consisting of SEQ ID NOs: 1-4 and 109-113 and a CDR2 comprising or consisting of an amino acid sequence selected from the group consisting of SEQ ID NOs: 5-8 and 114-117. Further, in some embodiments, the V_{L} of the antibody may comprise (a) a CDR1 comprising or consisting of an amino acid sequence selected from the group consisting of SEQ ID NOs: 16-19 and 130-131, (b) a CDR2 comprising or consisting of an amino acid sequence selected from the group consisting of SEQ ID NOs: 21-22 and 132-137, or (c) both a CDR1 comprising or consisting of an amino acid sequence selected from the group consisting of SEQ ID NOs: 16-19 and 130-131 and a CDR2 comprising or consisting of an amino acid sequence selected from the group consisting of SEQ ID NOs: 21-22 and 132-137.

In certain embodiments, the antibodies of the present invention comprise a CDR1, CDR2, and CDR3 that are all derived from a single variable region of an antibody disclosed herein. Specifically, in some embodiments, the V_{H} of the antibodies comprise the CDR1, CDR2, and CDR3 of a V_{H} having an amino acid sequence selected from the group consisting of SEQ ID NOs: 10-15 and 119-129. Further, in some embodiments, the V_{L} of the antibodies comprise the CDR1, CDR2, and CDR3 of a V_{L} having an amino acid sequence selected from the group consisting of SEQ ID NOs: 24-35 and 141-151. In some embodiments, the antibodies comprise all six CDRs (i.e., CDR1, CDR2, and CDR3 of the V_{H} and CDR1, CDR2, and CDR3 of the V_{L}) from a single antibody disclosed herein. Such antibodies comprise the paratope (i.e., a set of six CDRs that form an antigen-binding region) of an antibody disclosed herein.

Additionally, the present invention provides antibodies that comprise or consist of a full-length variable region disclosed herein. Specifically, in some embodiments, the antibodies comprise a V_{H} selected from the group consisting of SEQ ID NOs: 10-15 and 119-129. Further, in some embodiments, the antibodies comprise a V_{L} selected from the group consisting of SEQ ID NOs: 24-35 and 141-151.

In some embodiments, the antibodies of the present invention comprise at least a portion (e.g., CDR3) of both a V_{H} of an antibody disclosed herein and a V_{L} of an antibody disclosed herein. For example, in some embodiments, the antibodies disclosed herein comprise both a V_{H} selected from the group consisting of SEQ ID NOs: 10-15 and 119-129 and a V_{L} selected from the group consisting of SEQ ID NOs: 24-35 and 141-151. In some embodiments, the V_{H} and V_{L} are from a single antibody disclosed herein (see Table 1 and Table 3 for the V_{H} and V_{L} sequences of each antibody disclosed herein). In other embodiments, the V_{H} and the V_{L} are from two different antibodies disclosed herein.

In some embodiments, the antibodies comprise one or more amino acid modifications. As used herein, the term “amino acid modification” refers to a change in a polypeptide sequence. Amino acid modifications include deletions, additions, and substitutions of one or more amino acid residues. The antibodies of the present invention may comprise any combination of amino acid modifications so long as they retain the ability to bind APC with minimal to no binding to PC. In some embodiments, the antibody comprises 1, 2, 3, 4, 5, 6, 7, 8, 9, 10, 11, 12, 13, 14, 15, 16, 17, 18, 19, or 20 or more amino acid modifications. For example, in some embodiments, the antibody comprises a variant of a V_{H} selected from SEQ ID NOs: 10-15 and 119-129 comprising 1, 2, 3, 4, 5, 6, 7, 8, 9, 10, 11, 12, 13, 14, 15, 16, 17, 18, 19, or 20 amino acid modifications relative to the parent antibody. In other embodiments, the antibody comprises a V_{L} selected from SEQ ID NOs: 24-35 and 141-151 comprising 1, 2, 3, 4, 5, 6, 7, 8, 9, 10, 11, 12, 13, 14, 15, 16, 17, 18, 19, or 20 amino acid modifications relative to the parent antibody. In other embodiments, the antibody comprises a CDR3 selected from SEQ ID NOs: 9, 23, 118, and 138-140 comprising at least 1 amino acid modification.

In some embodiments, one or more of the amino acid modifications are conservative substitutions. As used herein, the term “conservative substitution” refers to an amino acid substitution that substantially conserves the structure and the function of the native polypeptide. Specifically, conservative substitutions generally maintain (a) the structure of the polypeptide backbone around the substitution (e.g., as a beta sheet or alpha helix), (b) the charge or hydrophobicity of the molecule at the site of the substitution, and/or (c) the bulk of the side chain. Examples of conservative substitutions are shown in Table 5.

TABLE 5
List of conservative amino acid substitutions

| Original reside | Conservative substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

In some embodiments, the antibodies of the present invention comprise a V_{H} with an amino acid sequence that has at least 99, 98, 97, 96, 95, 94, 93, 92, 91, 90, 85, 80, or 75 percent sequence identity to one of the amino acid sequences of SEQ ID NOs: 10-15 and 119-129, or that comprises a CDR3 having at least 99, 98, 97, 96, 95, 94, 93, 92, 91, or 90 percent sequence identity to one of the amino acid sequences of SEQ ID NOs: 9 and 118. In some embodiments, the antibodies of the present invention comprise a V_{L} with an amino acid sequence that has at least 99, 98, 97, 96, 95, 94, 93, 92, 91, 90, 85, 80, or 75 percent sequence identity to one of the amino acid sequences of SEQ ID NOs: 24-35 and 141-151, or that comprises a CDR3 having at least 99, 98, 97, 96, 95, 94, 93, 92, 91, or 90 percent sequence identity to one of the amino acid sequences of SEQ ID NOs: 23 and 138-140. In some embodiments, the antibodies of the present invention comprise a heavy chain with an amino acid sequence that has at least 99, 98, 97, 96, 95, 94, 93, 92, 91, 90, 85, 80, or 75 percent sequence identity to one of the amino acid sequences of SEQ ID NOs: 36-41 and 152-162. In some embodiments, the antibodies comprise a heavy chain that consists of one of the amino acid sequences of SEQ ID NOs: 36-41 and 152-162. In some embodiments, the antibodies of the present invention comprise a light chain with an amino acid sequence that has at least 99, 98, 97, 96, 95, 04, 93, 92, 91, 90, 85, 80, or 75 percent sequence identity to one of the amino acid sequences of SEQ ID NOs: 42-53 and 163-173. In some embodiments, the antibodies comprise a light chain that consists of one of the amino acid sequences of SEQ ID NOs: 42-53 and 163-173.

“Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100. Protein and nucleic acid sequence identities can be determined using the Basic Local Alignment Search Tool (“BLAST”), which is well known in the art (Proc. Natl. Acad. Sci. USA (1990) 87:2267-2268; Nucl. Acids Res. (1997) 25:3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula (Proc. Natl. Acad. Sci. USA (1990) 87:2267-2268). The BLAST programs can be used with the default parameters or with modified parameters provided by the user.

The present invention further provides antibodies that compete with an antibody described herein for binding to APC. Like the other antibodies described herein, these antibodies specifically bind to APC and minimally bind to PC. An antibody is said to “compete” with the binding of another antibody for a particular epitope if binding of one antibody results in decreased binding of the other antibody. Competition can occur either because the antibodies bind to the same epitope, or because the binding of one antibody interferes sterically with the binding of the other antibody or causes a conformational change that interferes with the binding of the other antibody. In some cases, a first antibody can inhibit the binding of a second antibody to its epitope without the second antibody inhibiting the binding of the first antibody to its epitope. However, in cases where both the first and the second antibody detectably inhibit the binding of the other antibody (whether to the same, greater, or lesser extent) the antibodies are said to “cross-compete” with each other for binding of their epitope(s). Antibodies that compete with or cross-compete with an antibody described herein for binding to APC are encompassed by the present invention.

### Nucleic Acids, Vectors, and Host Cells:

An antibody of the present invention can be produced by introducing a nucleic acid encoding the antibody into a host cell and providing suitable conditions for protein expression. Thus, in a second aspect, the present invention provides nucleic acids encoding the antibodies disclosed herein. Specifically, the invention includes nucleic acids encoding (1) an antibody comprising a V_{H} comprising or consisting of an amino acid sequence selected from SEQ ID NOs: 10-15 and 119-129, (2) an antibody comprising a V_{L} comprising or consisting of an amino acid sequence selected from SEQ ID NOs: 24-35 and 141-151, (3) an antibody comprising a CDR3 comprising or consisting of an amino acid sequence selected from SEQ ID NOs: 9, 23, 118, and 138-140, or (4) any other antibody described herein. The invention further includes nucleic acids comprising or consisting of a DNA sequence provided in Table 2 or Table 4 (i.e., a DNA sequence selected from SEQ ID NOs: 54-102 and 174-240) and variants thereof.

The terms “nucleic acid,” “polynucleotide,” and “oligonucleotide” are used interchangeably to refer to a polymer of DNA or RNA, which may be single-stranded or double-stranded, synthesized, or obtained (e.g., isolated and/or purified) from natural sources. Nucleic acids may contain natural, non-natural, or altered nucleotides, and may contain natural, non-natural, or altered internucleotide linkages (e.g., phosphoroamidate or phosphorothioate linkages). In some embodiments, the nucleic acids of the present invention are “isolated,” meaning that they are separated away from other cellular materials.

In a third aspect, the present invention provides vectors comprising the nucleic acids disclosed herein. The term “vector” refers to a DNA molecule that is used to carry a particular DNA segment (i.e., a DNA segment included in the vector) into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product (e.g., a promoter). Vectors may also include a reporter gene and/or a selectable marker gene. Suitable vectors include plasmids (i.e., circular double-stranded DNA molecules) and mini-chromosomes. Vectors suitable for use with the present invention comprise a DNA segment encoding an antibody described herein and a heterogeneous sequence that allows for expression of the encoded antibody.

In a fourth aspect, the present invention provides host cells comprising the nucleic acids and vectors disclosed herein. The term “host cell” is meant to refer to a transgenic cell in which heterologous DNA can be expressed. The nucleic acids or vectors disclosed herein may be introduced into a host cell using standard techniques including, for example, electroporation, heat shock, lipofection, microinjection, and particle bombardment.

In a fifth aspect, the present invention also provides methods of producing an antibody using the host cells disclosed herein. The methods comprise: (a) culturing a host cell disclosed herein under conditions that result in production of the antibody, and (b) isolating the antibody from the host cell. In these methods, antibodies are produced by culturing host cells for a sufficient period of time to allow for expression of the antibody in the host cells. Antibodies can then be recovered from the cell culture using standard protein purification methods, such as ultrafiltration, affinity chromatography, size exclusion chromatography, ion exchange chromatography, and centrifugation. Methods for expressing and purifying proteins are well known in the art (see, e.g., Nat Methods 5 (2): 135-146, 2008).

### Pharmaceutical Compositions:

In sixth aspect, the present invention provides pharmaceutical compositions comprising a therapeutically effective amount of an antibody disclosed herein and a pharmaceutically acceptable carrier. “Pharmaceutically acceptable” carriers are known in the art and include, but are not limited to, diluents, preservatives, solubilizers, emulsifiers, liposomes, nanoparticles, and adjuvants. Pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, and suspensions, including saline and buffered media.

The compositions of the present invention may further include diluents of various pH, ionic strength, and buffer content (e.g., Tris-HCl, acetate, phosphate), additives to prevent absorption to surfaces, detergents (e.g., Tween 20, Tween 80, Pluronic F68), solubilizing agents (e.g., glycerol, polyethylene glycerol), antioxidants (e.g., ascorbic acid, sodium metabisulfite, L-methionine), bulking substances, or tonicity modifiers (e.g., sucrose, mannitol). Within the compositions, the antibodies may be covalently attached polymers (e.g., polyethylene glycol), complexed with metal ions, or incorporated into or onto particulate preparations of polymeric compounds (e.g., polylactic acid, polyglycolic acid, hydrogels) or onto liposomes, microemulsions, micelles, multilamellar vesicles, erythrocyte ghosts, or spheroplasts. Inclusion of such compounds in the compositions will influence the physical state, solubility, stability, rate of in vivo release, and rate of in vivo clearance. The compositions may also be formulated in lipophilic depots (e.g., fatty acids, waxes, oils) for controlled or sustained release.

### Methods:

In a seventh aspect, the present invention provides methods for treating or preventing a condition. The methods comprise administering a therapeutically effective amount of an antibody or pharmaceutical composition disclosed herein to a subject.

The antibodies of the present invention can, optionally, be administered in combination with an exogenous APC protein to confer additional properties that cannot be achieved via antibody binding to the endogenous APC protein. Thus, in some embodiments, the methods comprise administering an exogenous APC protein or a variant thereof (e.g., 3K3A-APC) that is specifically bound to one or more of the antibodies disclosed herein to a subject.

As used herein, the term “treating” describes the management and care of a patient for the purpose of combating a condition. Treating includes the administration of an antibody or pharmaceutical composition of the present invention to alleviate the symptoms or complications of the condition or to eliminate the condition. As used herein, the term “condition” is used to refer to a health problem with certain characteristics and/or symptoms. The term condition is meant to encompass diseases, disorders, syndromes, and the like.

As used herein, the term “preventing” describes the management and care of a patient for the purpose of preventing the onset of symptoms or complications of a condition.

As used herein, the term “administering” refers to a method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, intravenous administration, intra-arterial administration, intramuscular administration, intradermal administration, intrathecal administration, and subcutaneous administration. Administration can be continuous or intermittent. In some embodiments, administration is systemic rather than local.

The term “therapeutically effective amount” refers to an amount that is sufficient to effect beneficial or desirable biological or clinical results. That result can be reducing, alleviating, inhibiting, or preventing one or more symptoms of a condition, or any other desired alteration of a biological system. For example, in some embodiments, a therapeutically effective amount is an amount suitable to promote blood clot formation. In other embodiments, a therapeutically effective amount is an amount suitable to treat sepsis. Methods for determining a therapeutically effective amount are well known to those of skill in the art. A therapeutically effective amount will vary with several factors including, for example, the formulation of the composition used for therapy, the purpose of the therapy, and the subject being treated. A therapeutically effective amount of a composition may be delivered via single or multiple administrations. For example, a suitable daily dosage may be in the range of 3-20 mg/patient per day, 1-3 mg/patient per day, 20-100 mg/patient per day, or 20-50 mg/patient per day.

In some embodiments, the methods of the present invention are used to treat or prevent a condition that can be treated or prevented by enhancing inhibiting APC's anticoagulant function. For example, in some embodiments, the methods are used to treat a condition in which blood clotting is desirable by inhibiting APC's anticoagulant function. Suitable conditions in which blood clotting is desirable include, without limitation, a hemorrhage (e.g., an intracranial hemorrhage, diffuse alveolar hemorrhage, intracerebral hemorrhage), a contusion (e.g., a brain contusion), a burn, gastrointestinal bleeding, uncontrolled bleeding, bleeding due to a transplantation (e.g., a stem cell transplantation, liver transplantation) or resection procedure, bleeding due to a surgery (e.g., a cardiac, spinal, orthopedic, neuro, oncological, or post-partum surgery), variceal bleeding, thrombocytopenia, idiopathic thrombocytopenia purpura, hemophilia, aortic aneurysm, reversal of an anticoagulant or antithrombotic (e.g., warfarin, heparin), bleeding due to a traumatic injury (e.g., a penetrating or blunt traumatic injury), menorrhagia, bleeding in cirrhosis (e.g., active variceal or non-variceal), deficiency of a clotting factor (e.g., factor VII), Glanzmann's thrombasthenia (e.g., refractory to platelet transfusion), and Bernard-Soulier syndrome. In some embodiments, the condition is an acute bleeding disorder. In some embodiments, the condition is an inherited bleeding disorder.

In other embodiments, the methods of the present invention are used to treat or prevent a condition that can be treated or prevented by enhancing or inhibiting one or more of APC's cytoprotective functions. For example, in some embodiments, the methods may be used to treat or prevent sepsis (Biochem Soc Trans 43:691-5, 2015), inflammation in acute ischemic disease (e.g., via providing neuroprotection in ischemic stroke (Ann Neurol 85:125-136, 2019) or providing cardioprotection in ischemic heart disease or heart failure (Int J Mol Sci 20:1762-1774, 2019)), coronavirus disease 2019 (COVID-19), diabetes (e.g., type 1 diabetes (J Biol Chem 287:16356-16364, 2012), diabetic nephropathy (Proc Natl Acad Sci USA 110:648-653, 2013; Nature Med 13:1349-1358, 2007; J Thromb Haemost 10:337-346, 2012; Blood 119:874-883, 2012), diabetic ulcers, wounds (Am J Pathol 179:2233-2242, 2011; Wound Repair Regen 13:284-294, 2005; Circ Res 95:34-41, 2004; J Invest Dermatol 125:1279-1285, 2005; J Biol Chem 286:6742-6750, 2011; Clin Haemorheol Microcirc 34:153-161, 2006; Arch Dermatol 144:1479-1483, 2008; Intern J Low Extrem Wounds 10:146-151, 2011), amyotrophic lateral sclerosis (ALS) caused by a mutation that is SODI (J Clin Invest 119:3437-3449, 2009), and multiple sclerosis (Nature 451:1076-1081, 2008; J Immunol 191:3764-3777, 2013). In some embodiments, the methods may be used to treat or prevent central nervous system injury (e.g., spinal cord ischemia), ischemic stroke, Alzheimer's disease, acute kidney injury, a lung disorder (e.g., acute lung injury, acute respiratory distress syndrome), or acute pancreatitis (Zhao et al., Int. J. Mol. Sci. 2019, 20, 903 at p. 12 of 20). In some embodiments, the methods are used to treat or prevent a condition that is associated with histones or NETs, such as a cancer (e.g., breast cancer, lung cancer, colorectal cancer, pancreatic cancer, blood cancer, neurological cancer, cutaneous cancer) or an inflammatory or autoimmune disease (e.g., psoriasis, rheumatoid arthritis, systemic lupus erythematosus).

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice, rats) for research purposes. In other embodiments, the methods are used to treat commercially important farm animals (e.g., cows, horses, pigs, rabbits, goats, sheep, chickens) or companion animals (e.g., cats, dogs). In a preferred embodiment, the subject is a human.

In an eighth aspect, the present invention provides uses of the antibodies disclosed herein as a medicament. The medicament may be for the treatment or prevention of (1) a condition that can be treated or prevented by inhibiting APC's anticoagulant function, and/or (2) a condition that can be treated or prevented by enhancing one or more of APC's pleiotropic cytoprotective functions. Examples of such conditions are provided above.

The results of the functional assays provided in the examples can be used to determine if a particular anti-APC antibody described herein is useful for a particular indication. For example, antibodies that inhibit APC's anticoagulation activity while maintaining its cytoprotective activities are useful for treating disorders that require blood coagulation (e.g., acute bleeding). The importance of maintaining APC's cytoprotective activities for such indications is evidenced by Magisetty et al. (Blood (2022) blood.2021013119), discussed supra, in which the ability of two anti-APC antibodies to treat hemophilic arthropathy was assessed in a murine model. One of these antibodies, i.e., MAPC1591, inhibits APC's anticoagulant activity without inhibiting its cytoprotective activities. The second antibody, i.e., MPC1609, inhibits both the anticoagulant and cytoprotective activities of APC. The results of this study suggest that preserving APC's cytoprotective activities is useful for treating hemophilic arthropathy. Although no significant differences were observed between the ability of the two antibodies to inhibit APC's anticoagulant activity, administration of MAPC1591, and not MPC1609, markedly reduced the severity of hemophilic arthropathy.

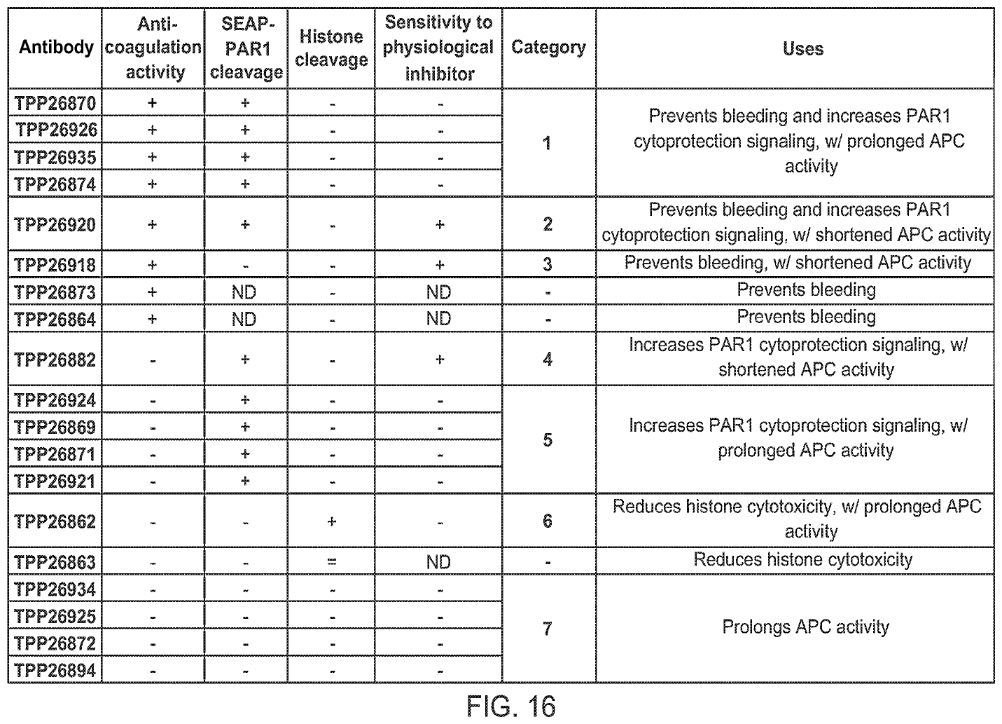

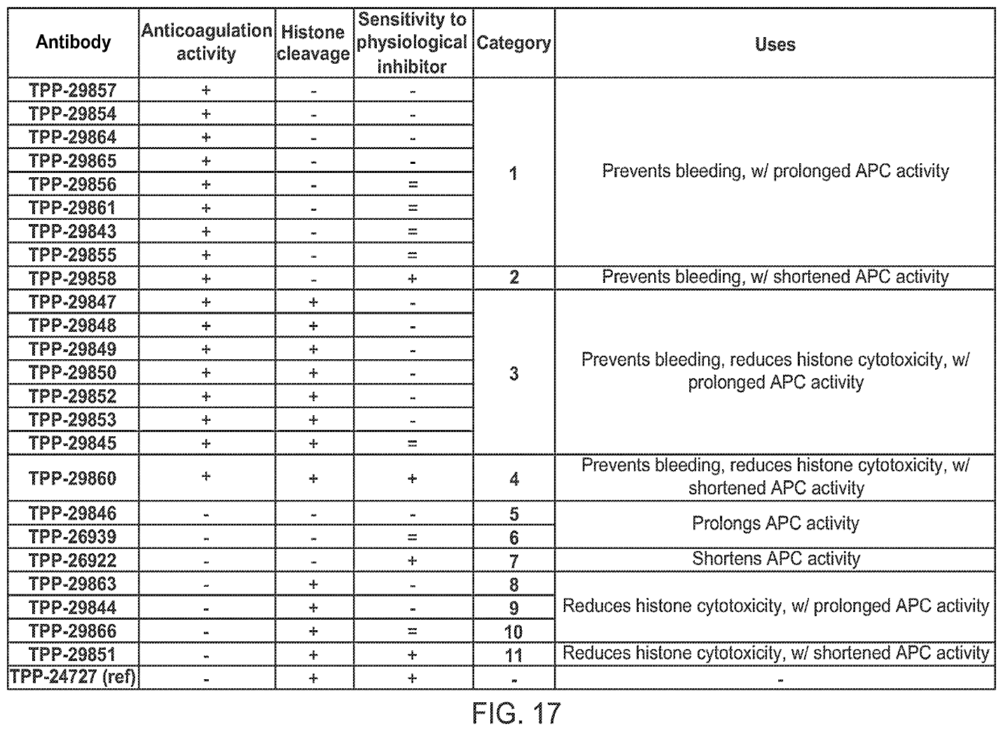

TPP-24727 is the parent antibody from which the antibodies of the present invention are derived. TPP-24727 inhibits APC's anticoagulation activity, enhances APC's histone H3 cleavage activity, and partially reduces APC-mediated PAR1 cleavage. In comparison to this parent antibody, the promising Set 1 antibody TPP-26870 more strongly inhibits APC anticoagulation activity, producing an enhanced pro-hemostatic effect. Further, TPP-26870 exhibits less inhibition of PAR1 cleavage than the parent antibody and does not interfere with APC-mediated histone cleavage. Anti-APC antibodies that provide improved cytoprotective effects in comparison to TPP-24727 or enhance APC's cytoprotective activities directly may also be useful for treating conditions such as sepsis and inflammation in acute ischemic disease. As is summarized in FIG. 16, the Set 1 antibodies can be divided into at least seven functional categories based on the pharmacological properties assayed in Example 3. Similarly, as is summarized in FIG. 17, the Set 2 antibodies can be divided into at least 11 functional categories based on the pharmacological properties assayed in Example 4.

### EXAMPLES

### Example 1—Optimization of the Parental Antibody

A previous study (Nat Commun 11 (1): 2992, 2020) reported two antibodies against APC that were shown to increase thrombin generation and promote plasma clotting in monkeys. One of these antibodies, referred to as TPP-4885, was well tolerated in monkeys and was determined to target an exosite on the APC protein. However, this antibody still partially inhibited the active site of APC and may therefore partially inhibit the cytoprotective effects of this enzyme.

The following Example describes the methods used to optimize TPP-4885. First, this IgG2 antibody was reformatted into an IgG4 antibody and renamed TPP-24727. An IgG Kappa S228P format was used, which contains a serine 228 to proline (S228P) mutation that serves to prevent Fab-arm exchange. Reformatting was performed using standard cloning techniques. Next, stretches of the framework sequence from TPP-24727 were exchanged for human germline sequences, i.e., KV2-28, KV3-20, KV3-11, V3-48-01.1, V3-15-01.1, and V1-69-04.1, to produce antibody frameworks that are more germline-like. Additionally, a deamidation site comprising two asparagine residues was removed from CDR2 of the heavy chain variable region (V_{H}) in a subset of the constructs (i.e., TPP-24737, TPP-24738, TPP-24739, TPP-24743, TPP-24745, TPP-24750, TPP-24766, TPP-24769, TPP-24777, and TPP-24815). This exchange process generated 96 different antibody light chains and 96 different antibody heavy chains. The modified antibody chains were evaluated by pairing each modified chain with the other unmodified chain from the parent antibody (i.e., each modified light chain was paired with the unmodified heavy chain of TPP-24727 and each modified heavy chain was paired with the unmodified light chain of TPP-24727).

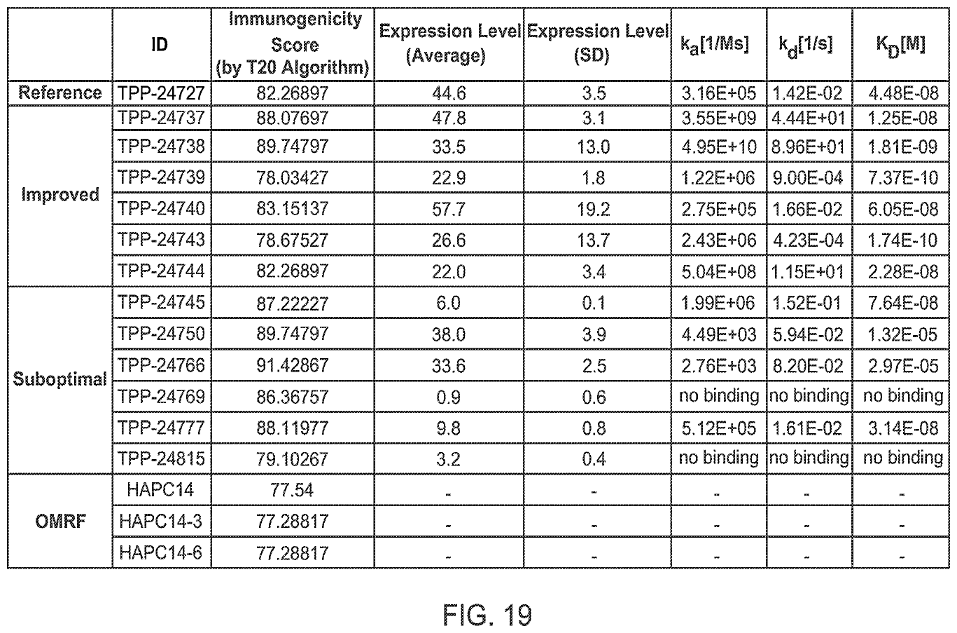

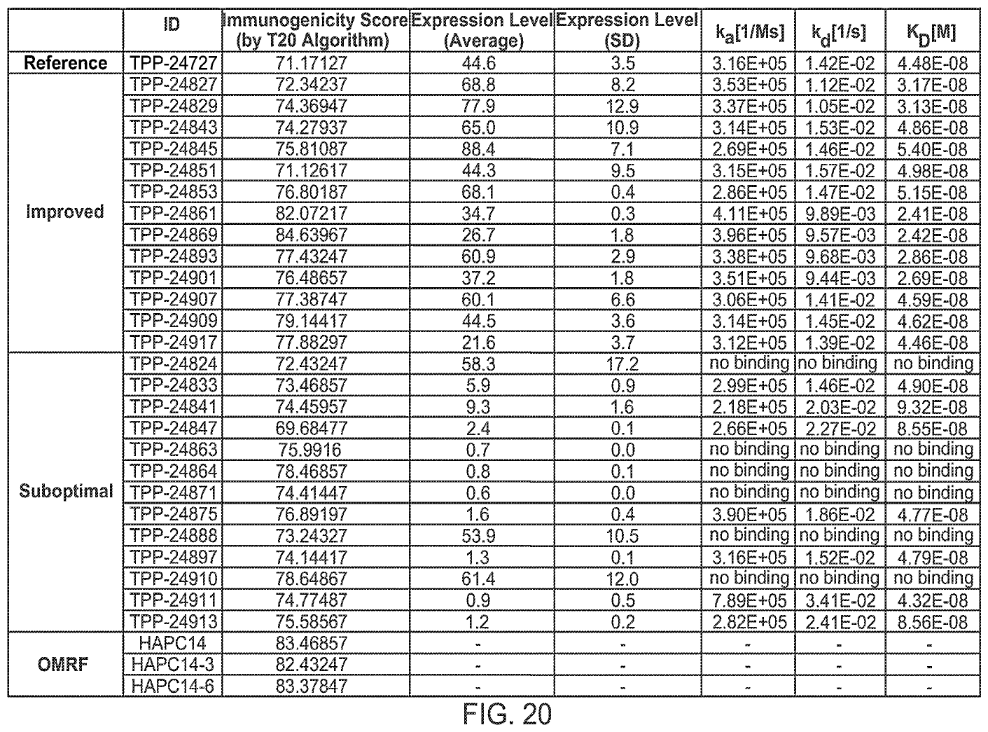

As is demonstrated in FIG. 19 and FIG. 20, the sequence exchanges produced several heavy chains and light chains with improved immunogenicity, expression levels, and/or K_{D} as compared to the parent antibody TPP-24727. The sequence identifiers associated with each of the heavy chains characterized in FIG. 19 are listed in Table 6, and the sequence identifiers associated with each of the light chains characterized in FIG. 20 are listed in Table 7.

TABLE 6
Sequence identifiers assigned to the amino acid sequences
of the CDRs and V_{H} of antibodies described in FIG. 19

| | V_{H} | | | |
|---|---|---|---|---|
| Antibody name | CDR1 | CDR2 | CDR3 | Full-length |
| TPP-24727 | 1 | 5 | 9 | 10 |
| TPP-24737 | 2 | 6 | 9 | 11 |
| TPP-24738 | 2 | 7 | 9 | 12 |
| TPP-24739 | 2 | 8 | 9 | 13 |
| TPP-24740 | 3 | 5 | 9 | 14 |
| TPP-24743 | 3 | 8 | 9 | 250 |
| TPP-24744 | 4 | 5 | 9 | 15 |
| TPP-24745 | 4 | 6 | 9 | 251 |
| TPP-24750 | 243 | 7 | 9 | 252 |
| TPP-24766 | 244 | 7 | 9 | 253 |
| TPP-24769 | 245 | 6 | 9 | 254 |
| TPP-24777 | 246 | 6 | 9 | 255 |
| TPP-24815 | 247 | 8 | 9 | 256 |
| HAPC14 | 1 | 5 | 9 | 257 |
| HAPC14-3 | 248 | 249 | 9 | 258 |
| HAPC14-6 | 248 | 249 | 9 | 258 |

TABLE 7
Sequence identifiers assigned to the amino acid sequences
of the CDRs and V_{L} of antibodies described in FIG. 20

| | V_{L} | | | |
|---|---|---|---|---|
| Antibody name | CDR1 | CDR2 | CDR3 | Full-length |
| TPP-24727 | 16 | 21 | 23 | 24 |
| TPP-24827 | 16 | 22 | 23 | 263 |
| TPP-24829 | 16 | 22 | 23 | 35 |
| TPP-24843 | 20 | 22 | 23 | 30 |
| TPP-24845 | 20 | 22 | 23 | 33 |
| TPP-24851 | 20 | 22 | 23 | 31 |
| TPP-24853 | 20 | 22 | 23 | 32 |
| TPP-24861 | 17 | 22 | 23 | 25 |
| TPP-24869 | 17 | 22 | 23 | 26 |
| TPP-24893 | 18 | 22 | 23 | 264 |
| TPP-24901 | 18 | 22 | 23 | 27 |
| TPP-24907 | 19 | 22 | 23 | 28 |
| TPP-24909 | 19 | 22 | 23 | 29 |
| TPP-24917 | 19 | 22 | 23 | 34 |
| TPP-24824 | 16 | 21 | 261 | 265 |
| TPP-24833 | 16 | 21 | 23 | 266 |
| TPP-24841 | 20 | 21 | 23 | 267 |
| TPP-24847 | 20 | 21 | 23 | 268 |
| TPP-24863 | 17 | 21 | 23 | 269 |
| TPP-24864 | 17 | 21 | 261 | 270 |
| TPP-24871 | 259 | 21 | 23 | 271 |
| TPP-24875 | 259 | 22 | 23 | 272 |
| TPP-24888 | 18 | 21 | 261 | 273 |
| TPP-24897 | 18 | 21 | 23 | 274 |
| TPP-24910 | 19 | 22 | 261 | 275 |
| TPP-24911 | 19 | 21 | 23 | 276 |
| TPP-24913 | 19 | 21 | 23 | 277 |
| HAPC14 | 18 | 21 | 23 | 278 |
| HAPC14-3 | 18 | 260 | 262 | 279 |
| HAPC14-6 | 18 | 260 | 23 | 280 |

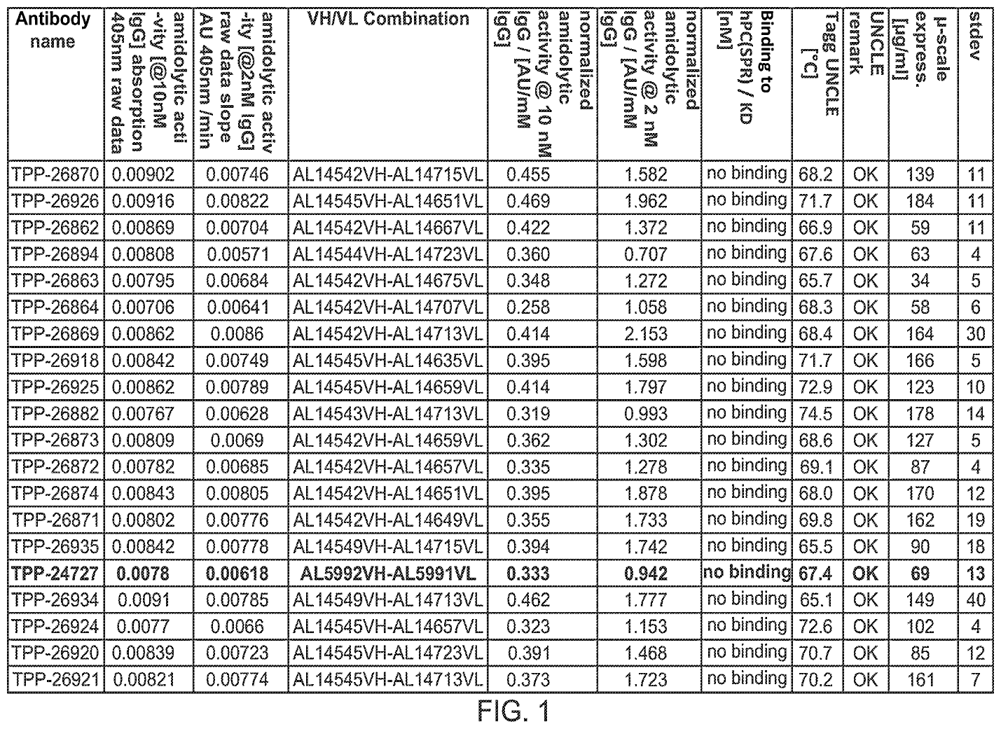

The expression levels in transiently transfected HEK293 cells, amidolytic activity (SPECTROZYME® PCa), binding to both human APC and PC (i.e., kon, koff, kD) based surface plasmon resonance (SPR), and aggregation temperature (Tagg) based on UNcle were tested for each modified antibody chain. Based on these measurements, 13 light chains and 6 heavy chains were selected and combined to generate 78 optimized antibodies. The 78 new antibodies were re-screened using the same assays, and 20 antibodies were selected for further study. These 20 optimized antibodies are referred to herein as the Set 1 antibodies. The results of the initial screening assays for the Set 1 antibodies are shown in FIG. 1.

The Set 1 antibodies include antibodies that possess diverse combinations of improved properties as compared to the parental antibody TPP-24727. They also include a diverse array of heavy and light chains, providing a wide optimization space for further affinity maturation. The sequences of the variable domains of the Set 1 antibodies are outlined in Table 1. Scaled-up production of the 20 Set 1 antibodies was performed in preparation for extended functional and chemistry, manufacturing, and control (CMC) evaluations, including those described in Example 2.

### Materials

Plasma-derived human APC and human PC were procured from Enzyme Research Laboratories (South Bend, IN, USA) and human APC substrate SPECTROZYME® PCa was procured from American Diagnostica (Pfungstadt, Germany).

### Expression and Purification

Genes encoding the antibody heavy and light chains were subcloned separately into the expression vector pTT5 (National Research Council, Canada) and co-transfected into HEK293 cells (National Research Council, Canada). The cells were maintained in FreeStyle™ F17 Expression medium (Invitrogen) supplemented with 2 mM GlutaMAX™ Supplement (Invitrogen), 0.1% Pluronic™ F-68 Non-ionic Surfactant (Sigma-Aldrich), and 0.5% PenStrep (Penicillin-Streptomycin solution) (Invivogen). After 5 days of transient expression, the cleared supernatants were affinity-purified using protein A beads (GE Healthcare) followed by buffer exchange into PBS using Zeba™ Spin desalting columns (Thermo Fisher). IgG concentrations were quantified via sandwich ELISA using anti-human Fc (Sigma) for capturing and anti-Human IgG (Fc specific)-Peroxidase antibody (Sigma) for detection with the Amplex™ Red hydrogen peroxide/peroxidase assay kit (Invitrogen). Human Reference Serum (Bethyl) was used as an IgG concentration standard.

### Aggregation Temperature

Aggregation onset temperature (Tagg) was determined using an UNcle device (an all-in-one biologics stability platform; UNCHAINED Labs) according to the manufacture's protocol. This machine was used to measure the static scattered light of protein solutions at 266 nm, and the onset of aggregation was calculated using the UNcle software. The supernatant was collected from HEK293 cells that were induced to express the antibodies of interest (see above) and was concentrated and buffer exchanged (to PBS) using a VIVASpin® 500 centrifugal concentrator device (10000 MWCO PES, Sartorius). Final protein solutions (<0.5 mg/mL in PBS buffer) were centrifuged for 2 minutes at 8000 g prior to analysis. The following temperature gradient was used for analysis: start temperature: 30° C., rate: 0.33° C./min, end temperature: 95° C.

### Affinity and Binding Kinetics

Surface plasmon resonance (SPR) assays were conducted to assess the binding kinetics and affinity of the anti-APC antibodies. Binding assays were performed on a Biacore™ T200 instrument (GE Healthcare) at 25° C. using assay buffer (HBS-P+, 1 mg/ml BSA, 0.01% Na-azide, 300 mM NaCl, 2.75 mM MgCl2, 0.75 mM CaCl2). Antibodies were captured using anti-human Fc IgG covalently coupled (via an amine) to a CM5 sensor chip. Human APC and PC were used as analytes at a concentration of 100 nM and 1 μM, respectively. To obtain reliable dissociation rate constants (k_{d}), the dissociation rate was set to 600 seconds after association of 180 seconds. The resulting sensorgrams were fitted to a 1:1 Langmuir binding model to derive kinetic and affinity data. The resulting sensorgrams were fitted to a 1:1 Langmuir binding model to derive kinetic and affinity data.

### Effect on APC's Amidolytic Activity

Amidolytic activity assays were performed to assess the effect of antibody binding on the amidolytic activity of human APC (hAPC). hAPC (20 nM) was combined in equal volumes with each monoclonal antibody (mAb) at various concentrations (20 nM, 4 nM, and 0.5 nM) in a 96-well dilution plate and pre-incubated for 20 minutes at room temperature. Thereafter, the APC/antibody mixtures were transferred to 384-well plates at 10 μL/well in quadruplicates, and 10 μL of the substrate SPECTROZYME® PCa (at 0.1 mM) was added to each well. Substrate hydrolysis was monitored at 37° C. using a Tecan Infinite® M1000Pro reader at 405 nm for chromogenic reading.

### Example 2—Screening of the Set 1 Antibodies

The following Example describes several assays that were used to evaluate the top 20 anti-APC antibodies identified in Example 1 (i.e., the Set 1 antibodies). The goal of these evaluations was to assess the effect of the antibodies on APC's anticoagulant activity and cytoprotective functions.

### Affinity and Binding Kinetics

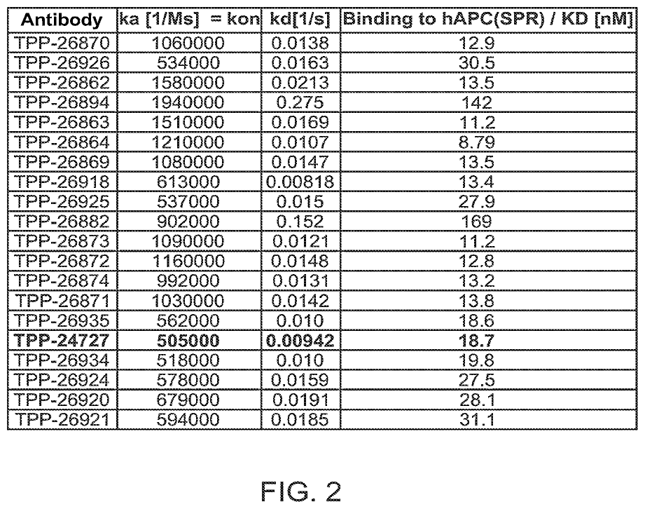

To assess the binding kinetics and affinity of the Set 1 antibodies for APC, binding assays were conducted using surface plasmon resonance (SPR). Binding assays were performed using a Biacore™ T200 instrument (GE Healthcare) at 25° C. using assay buffer (HBS-P+, 1 mg/ml BSA, 0.01% Na-azide, 300 mM NaCl, 2.75 mM MgCl₂, 0.75 mM CaCl₂)). Antibodies were captured by anti-human Fc IgG covalently coupled to a CM5 sensor chip. Human APC and PC were used as analytes, at concentrations of 100 nM and 1 μM, respectively. To obtain reliable dissociation rate constants (k_{d}) the dissociation rate was set to 600 seconds after association of 180 seconds. The resulting sensorgrams were fitted to a 1:1 Langmuir binding model to derive the kinetic and affinity data presented in FIG. 2. All 20 Set 1 antibodies showed improved ka, 1 antibody showed improved k_{d}, and 11 antibodies showed improved K_{D} over the parental antibody TPP-24727.

### Protein Concentration

IgG concentrations were quantified via sandwich ELISA using anti-human Fc (Sigma) for capturing and anti-human IgG (Fc specific) peroxidase antibody (Sigma) for detection with an Amplex™ Red hydrogen peroxide/peroxidase assay kit (Invitrogen). Human Reference Serum (Bethyl) was used as an IgG concentration reference.

### Aggregation Temperature and Melting Temperatures

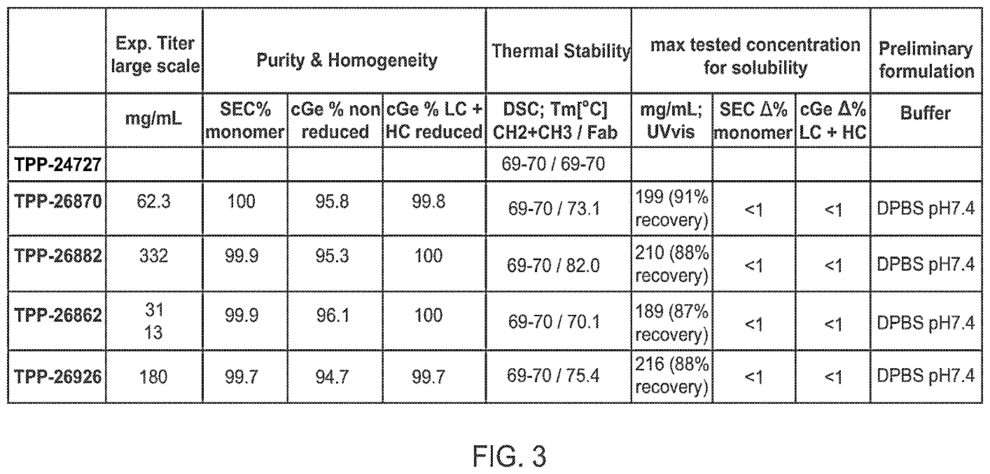

Aggregation onset temperature (Tagg) was determined using an UNcle device (UNCHAINED Labs) according to the manufacture's protocol. This machine was used to measure the static scattered light of protein solutions at 266 nm, and the onset of aggregation was calculated using the UNcle software. Additionally, melting temperatures (Tm) were determined via differential scanning calorimetry using a MicroCal VP-Capillary DSC device, according to the manufacture's protocol. Reference protein solutions (0.5 mg/mL in PBS, pH 7.4) were centrifuged for 2 minutes at 8000 g prior to analysis. The following temperature gradient was used for analysis: start temperature: 25° C., scan rate for heating: 2° C./min, end temperature: 95° C. Heat capacity signals were deconvoluted to yield underlying peaks in case of different melting temperatures (for CH2/CH3 and Fab). HEK293 supernatants containing the antibodies from transient expressions (see above) were concentrated and buffer was exchanged (to PBS) using a VIVASpin® 500 centrifugal concentrator device (10000 MWCO PES, Satorius). Final protein solutions (<0.5 mg/mL in PBS buffer) were centrifuged for 2 minutes at 8000 g prior to analysis. For the analysis, the following temperature gradient was used: start temperature: 30° C., rate: 0.33° C./min, end temperature: 95° C. The results of this analysis are presented in FIG. 3. Of the 20 Set 1 antibodies, all were shown to be of high purity and percent monomer, and to have a solubility of greater than 188 mg/mL. Among them, TPP-26882 has the highest expression titer (332 mg/mL) and thermal stability (Tm=82.0° C.).

### Effect on APC's Amidolytic Activity

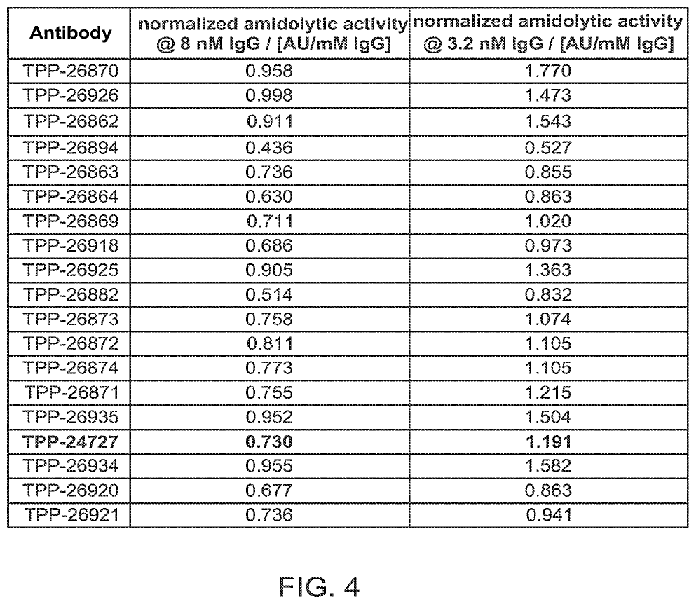

To assess the effect of the Set 1 antibodies on the amidolytic activity of APC, 20 nM human APC was combined at equal volumes with the 20 antibodies at different concentrations in a 96-well plate and pre-incubated for 20 minutes at room temperature. Thereafter, the APC/antibody mixture was transferred to 384-well assay plates at 10 μL/well in quadruplicates, and 10 μL of the substrate SPECTROZYME® PCa (0.1 mM) was added. Substrate hydrolysis was monitored at 37° C. and 405 nm using a Tecan Infinite® M1000Pro reader for chromogenic reading. The results of this assay are presented in FIG. 4. The anti-APC antibodies were tested at 8 nM and 3.2 nM. At 8 nM, 12 antibodies led to higher amidolytic activity as compared to the parental antibody TPP-24727. At 3.2 nM, 7 antibodies led to higher activity than TPP-24727.

### FVa Inactivation

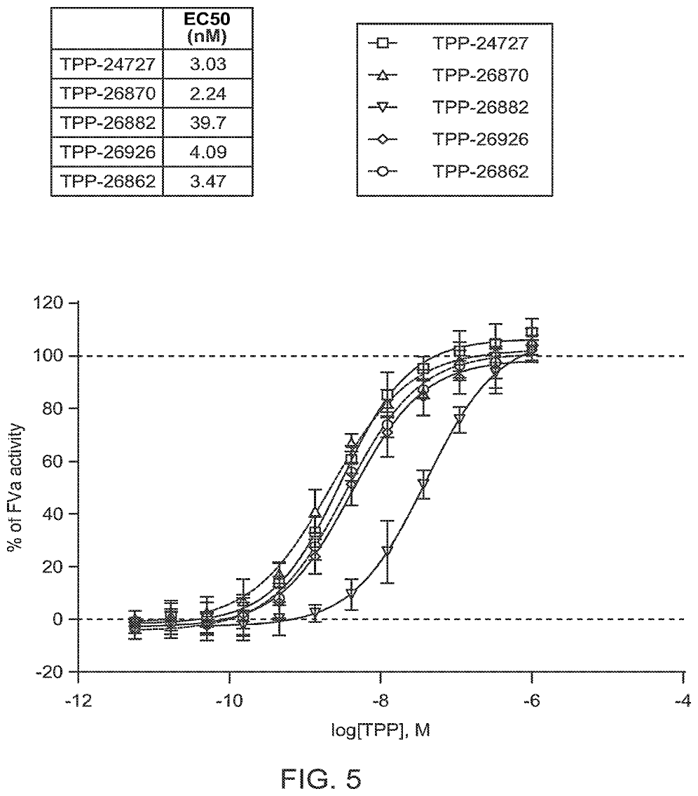

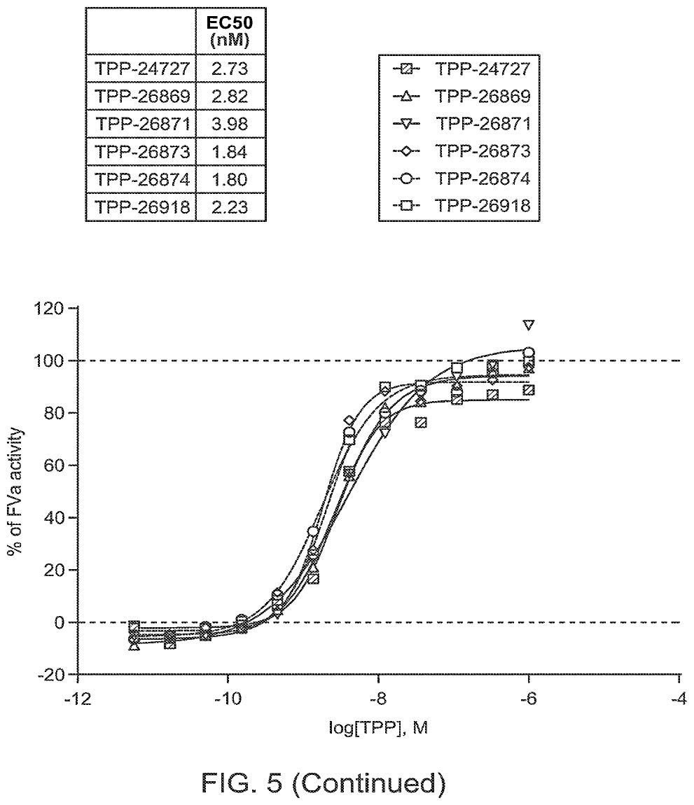

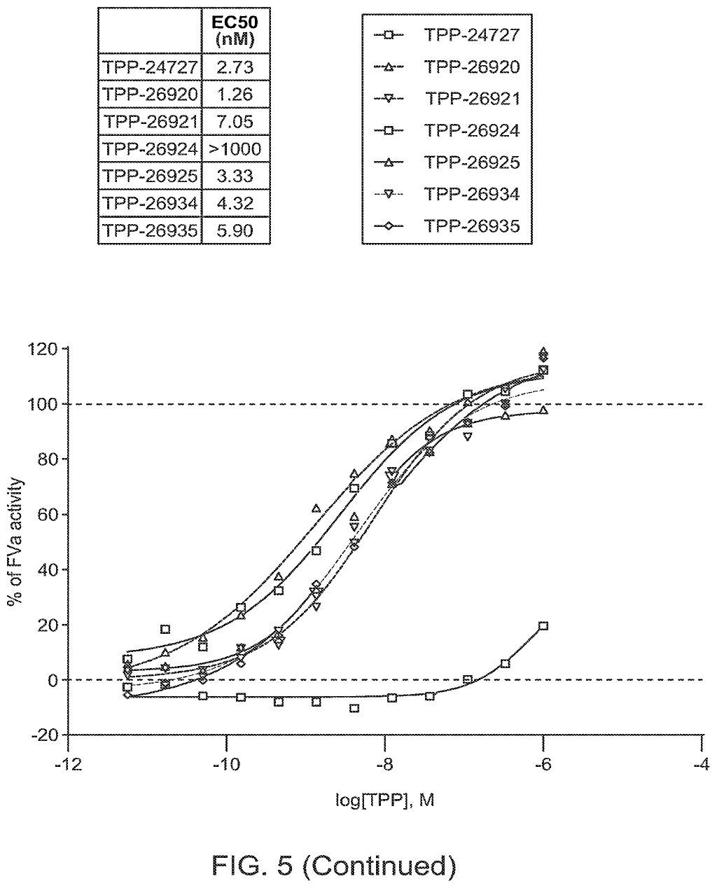

The ability of the Set 1 antibodies to inhibit APC's inactivation of FVa was measured using a FXa- and thrombin-generation assay. In this assay, 20 PM FVa was incubated with 50 pM APC in assay buffer (25 mM Tris-HCl, 130 mM NaCl, 2.7 mM KCl, 25 μM phospholipids (PC:PE:PS 40:40:20), 5 mM CaCl₂), and 1 mg/mL BSA) with each antibody at concentrations ranging from 0.5 to 1000 nM. After a 30-minute incubation at room temperature, 100 μL of prothrombinase mixture containing 400 nM FXa, 1 μM prothrombin, and 0.6 mM thrombin chromogenic substrate (S-2238) was added to the 100-μL incubation mixture. The kinetics of thrombin-mediated substrate hydrolysis was monitored at 405 nm at 25° C. using a plate reader for 3 hours. The results of this assay are presented in FIG. 5. Among the tested antibodies, TPP-26783, TPP-26784, and TPP-26920 showed improved EC50 in inhibiting FVa as compared to the parental antibody TPP-24727.

### Example 3: Functional Assessment of the Set 1 Antibodies

In the following Example, the top 20 anti-APC antibodies identified in Example 1 (i.e., the Set 1 antibodies) are functionally characterized using several assays. Specifically, the effects of the antibodies on the APC's coagulation-related activities are assessed using a plasma clotting assay; the effects of the antibodies on the cytoprotective activities of APC are assessed using a PAR1 cleavage assay, an endothelial barrier integrity assay, and a histone H3 cleavage assay; and the effects of the antibodies on the sensitivity of APC to physiological inhibitors are assessed using an in vitro plasma half-life assay.

The results of these functional assays will aide in determining if a particular Set 1 antibody is useful for a particular indication. For example, antibodies that inhibit APC's anticoagulation activity while maintaining its cytoprotective activities are useful for treating disorders that require blood coagulation (e.g., acute bleeding), whereas antibodies that enhance APC's cytoprotective activities may be useful for treating disorders such as sepsis and inflammation in acute ischemic disease.

### Results:

### Clotting Time

The ability of the Set 1 antibodies to decrease clotting time was measured using a Protac®-APTT clotting assay. The results of this assay revealed that the antibodies reduce in vitro clotting time to varying degrees (FIG. 6). The antibodies TPP-26870, TPP-26935, TPP-26874, TPP-26873, TPP-26926, TPP-26864, TPP-26918, and TPP-26920 were shown to have improved potency for inhibiting APC's anticoagulant effects and reducing plasma clotting time as compared to the parental antibody TPP-24727. Of these antibodies, TPP-26870 showed the greatest reduction in clotting time.

### PAR1 Cleavage

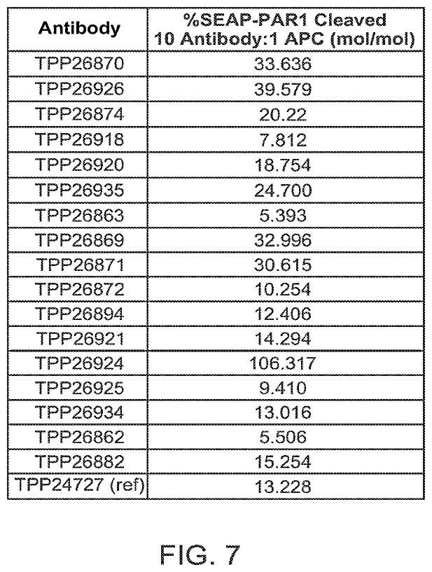

Cleavage and activation of protease-activated receptor 1 (PAR1) by APC alone induces cytoprotective effects, including protective gene expression, enhanced endothelial barrier function, reduced cytokine secretion, and protection against apoptosis. Thus, the ability of the Set 1 antibodies to inhibit APC-mediated PAR1 cleavage was tested using a secreted embryonic alkaline phosphatase (SEAP)-protease-activated receptor 1 (PAR1) cleavage assay. The results of this assay are presented in FIG. 7. Based on these results, antibodies with an increased (e.g., TPP-26918) or reduced (e.g., TPP-26920) ability to inhibit APC-mediated PAR1 cleavage were identified, as well as one antibody (i.e., TPP-26924) that did not show any inhibition of APC-mediated PAR1 cleavage at concentrations of up to 500 nM.

### Endothelial Barrier Integrity

APC has a protective effect on endothelial barrier function. Thus, the effect of the Set 1 antibodies on this cytoprotective activity were tested using an endothelial barrier integrity assay. The results of this assay are presented in FIG. 8. Several antibodies that enhance APC-mediated endothelial barrier protection (e.g., TPP26926 and TPP26935) were identified based on these results.

### Histone Cleavage

Histones act as damage-associated molecular patterns (DAMPs) when they are released into the extracellular space, leading to systemic inflammation and toxic responses. APC inhibits histone-induced cytotoxicity via proteolytic cleavage of histones. Thus, the ability of the Set 1 antibodies to inhibit APC-mediated histone cleavage was tested using a histone H3 cleavage assay. Based on the results of this assay, antibodies that could either inhibit or enhance APC-mediated H3 cleavage were identified (FIG. 9). For example, TPP-26921 inhibited cleavage while TPP-26862 enhanced both the rate of cleavage and the total amount of cleavage.

### Plasma Half-Life

An in vitro plasma half-life assay was used to determine the effect of the Set 1 antibodies on the sensitivity of APC to physiological inhibitors. This assay identified antibodies that increase the serum half-life of APC (FIG. 10). This increase in half-life indicates that the binding of these antibodies reduces the susceptibility of APC to inhibition by physiological inhibitors present in plasma.

### Materials and Methods:

Protac®-APTT clotting assay. Plasma samples (50 μL) containing different concentrations of the Set 1 antibodies were mixed with Protac® (25 μL; 1 U/mL) and STA-PTT reagent (75 μL) at 37° C. After a 4-minute incubation, 75 μL of 25 mM CaCl₂) solution was added to initiate clotting. In this assay, Protac® (a single chain glycoprotein derived from snake venom that activates protein C) is added to the plasma to activate protein C, reduce coagulation activity, and prolong clotting time.

SEAP-PAR1 cleavage assay. A PAR1 cleavage reporter construct was made with an N-terminal SEAP domain. HEK293 cells expressing SEAP-PAR1 and wild-type (wt)-EPCR were grown in 96-well plates until confluent. On the day of the experiment, cells were washed with Hanks' balanced salt solution supplemented with 1.3 mM CaCl₂, 0.6 mM MgCl₂, and 0.1% BSA (HMM2). Plasma-derived APC (50 or 100 nM) and antibody were preincubated for 30 minutes in HMM2 before being added to confluent SEAP-PAR1/wt-EPCR cells in 96-well plates. After 60 minutes, SEAP release was measured using 1-step p-nitrophenyl phosphate (Pierce #37621). After correction for background activity in the absence of APC, values were expressed as the percentage of the total SEAP activity present on the cells. Values are normalized to the values obtained with no antibody (=100%).

Endothelial barrier integrity assay. Endothelial cell permeability was measured in real time using the iCELLigence™ system (ACEA, San Diego, CA), which measures changes in transendothelial electrical resistance (TEER) by electric cell-substrate impedance sensing (ECIS). Briefly, EA.hy926 endothelial cells were grown to confluence for 2-3 days in an 8-well culture dish containing gold-film electrodes. DMEM (Dulbecco's Modified Eagle Medium; Invitrogen) containing 10% fetal calf serum was replaced with serum-free media containing 0.1% BSA 2 hours before addition of APC (50 nM) in the presence and absence of antibody. Permeability of confluent monolayers after treatment with APC for 3 hours was determined upon addition of thrombin (2 nM). All comparisons of the cell index were made using normalized resistance. The results were expressed as percentage of maximal barrier protection by APC in the absence of antibodies.

Histone H3 cleavage assay. APC (50 nM) and antibody (500 nM) were preincubated for 30 minutes in HBS buffer ((20 mM Hepes, 147 mM NaCl, 3 mM KCl)+100 μg/ml BSA+2 mM CaCl₂), pH 7.4). After the incubation, the mixtures were added to 100 μg/ml histone H3 (H3; Roche). Over a period of 2 hours, the mixtures were subsampled at different time points and added to reducing sample buffer. The subsamples were then heated for 15 minutes at 95° C., spun for 5 minutes at 4000 rpm, briefly vortexed, and loaded into a 12% Bis-Tris gel with MES running buffer (30 μl/well; 750 ng H3). Odyssey® marker (10 μL) was used as molecular weight standard with the 20 kDa marker as a reference for normalization between gels. Gels were stained overnight with Biotium One-Step Blue® Protein Gel Stain (50 ml/gel). Protein bands were scanned on LI-COR imaging system (700 channel, intensity 6, 1 mm offset, resolution 169 μm, quality highest, manual background). Images were analyzed using Image Studio V5.2.

In vitro plasma half-life assay. APC (70 nM) and antibody (700 nM) were preincubated for 30 minutes and normal pooled plasma was added to 90% (v/v). Samples were collected at various timepoints (0-90 minutes) and quenched in ice-cold TBS. The chromogenic activity of APC was determined using Pefachrome® PCa. Background chromogenic activity of plasma (without APC) was subtracted, and APC activity was normalized to the t=0 timepoint. Half-life (T_{1/2}) was determined by one-phase exponential decay curve fitting.

### Example 4—Screening and Functional Assessment of the Set 2 Antibodies

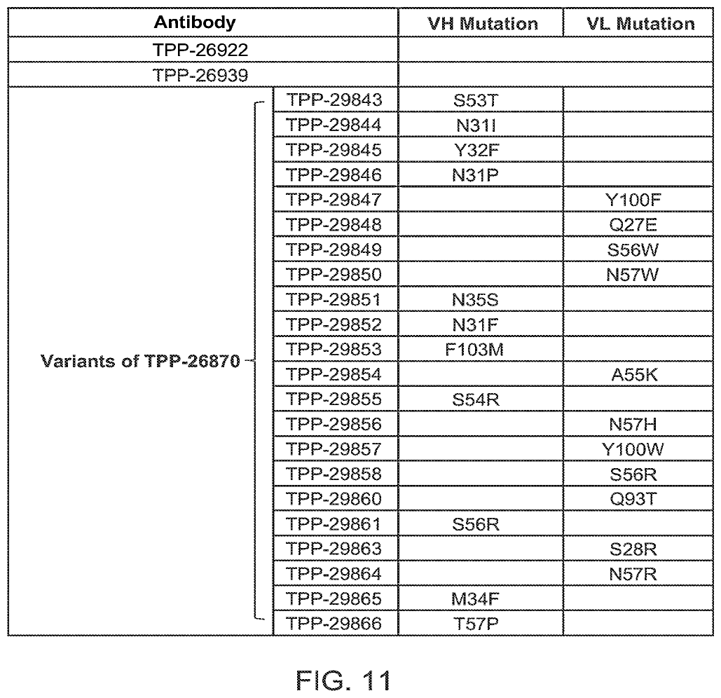

The following Example describes several assays that were used to evaluate a second set of 24 anti-APC antibodies, referred to herein as the Set 2 antibodies. The Set 2 antibodies include 22 variants of the promising Set 1 antibody TPP-26870 that have greater affinity for APC as compared to TPP-26870. These variants were generated by creating a single NNK site saturation mutagenesis library based on TPP-26870. The mutations found in each these 22 antibody variants are outlined in FIG. 11. Set 2 also includes two additional antibodies (i.e., TPP-26922 and TPP-26939) that are not related to any of the set 1 antibodies. The sequences of the variable domains of the Set 2 antibodies are outlined in Table 3.

Several assays were used to screen and assess the functional characteristics of the Set 2 antibodies. Again, the goal of these evaluations was to assess the effect of the antibodies on APC's anticoagulant activity and cytoprotective functions.

### Screening Assays

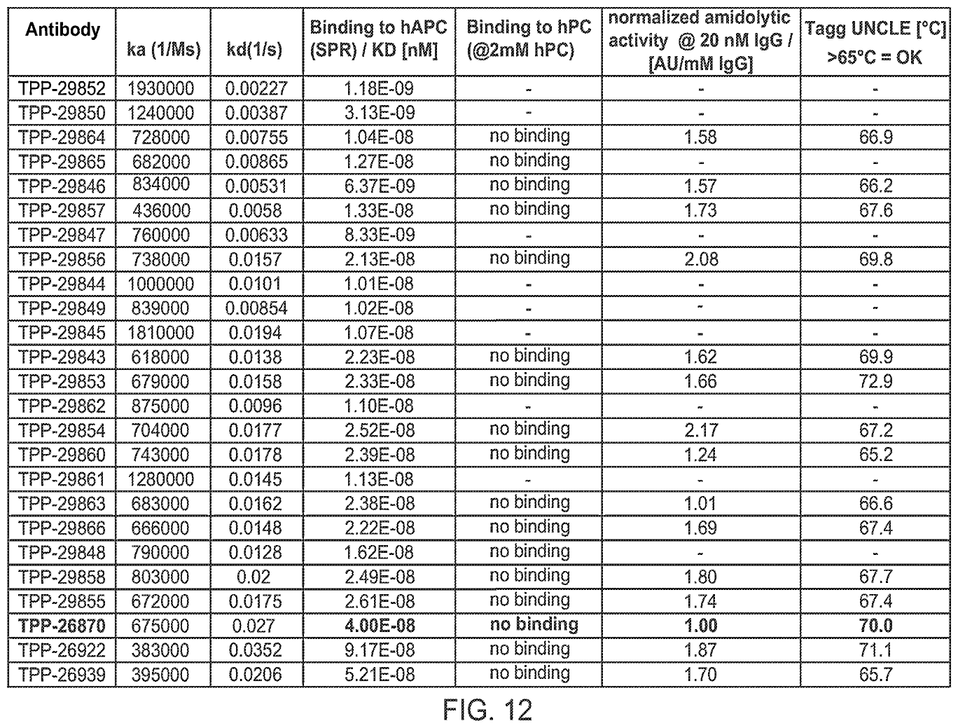

Several of the assays described in Example 2 were used to screen the Set 2 antibodies. Specifically, (1) surface plasmon resonance (SPR) was used to assess the binding kinetics and affinity of the antibodies for APC and PC, (2) amidolytic activity assays were used to assess the effects of the antibodies on APC's substrate cleavage activity, and (3) the aggregation onset temperatures (Tagg) of the antibodies were determined using an UNcle device. The results of these assays are presented in FIG. 12. Each of these assays was performed as described in Example 2.

### Clotting Time

The ability of the Set 2 antibodies to decrease clotting time was measured using a Protac®-APTT clotting assay. The results of this assay revealed that several of the TPP-26870 variant antibodies (i.e., TPP-29843, TPP-29847, TPP-29850, TPP-29852, TPP-29853, TPP-29854, TPP-29855, TPP-29856, TPP-29857, TPP-29858, TPP-29864, and TPP-29865) exhibit enhanced inhibition of APC's anticoagulation activity relative to the parent antibody and resulted in reduced in vitro clotting time, whereas others (e.g., TPP-29851, TPP-29844) exhibited reduced inhibition of APC anticoagulation activity (FIG. 13). The Protac®-APTT clotting assay was performed as described in Example 3.

### Histone Cleavage

The ability of the Set 2 antibodies to inhibit APC-mediated histone cleavage was assayed using a histone H3 cleavage assay. This assay identified antibodies that could either inhibit or enhance histone cleavage to varying degrees (FIG. 14). For example, TPP-29864 inhibited APC-mediated H3 cleavage while TPP-29848 enhanced the total amount of cleavage. The histone H3 cleavage assay was performed as described in Example 3.

### Plasma Half-Life

An in vitro plasma half-life assay was used to determine the effect of Set 2 antibodies on the sensitivity of APC to physiological inhibitors. The results of this assay were used to identified antibodies that increase the serum half-life of APC (FIG. 15). This increase in half-life indicates that the binding of these antibodies reduces the susceptibility of APC to inhibition by physiological inhibitors present in plasma. The in vitro plasma half-life assay was performed as described in Example 3.

### REFERENCES: ADDIN EN.REFLIST

Each patent and non-patent publication cited herein is hereby incorporated herein by reference in its entirety, including without limitation the references listed below.
1. Peyvandi, F., Garagiola, I. & Young, G. The past and future of haemophilia: diagnosis, treatments, and its complications. Lancet 388, 187-197 (2016).
2. Lusher, J. M. Inhibitor antibodies to factor VIII and factor IX: management. Semin. Thromb. Hemost. 26, 179-188 (2000).
3. Hartmann, J. & Croteau, S. E. 2017 clinical trials update: innovations in hemophilia therapy. Am. J. Hematol. 91, 1252-1260 (2016).
4. Esmon, C. T. Protein C anticoagulant system-anti-inflammatory effects. Semin. Immunopathol. 34, 127-132 (2012).
5. Griffin, J. H., Zlokovic, B. V. & Mosnier, L. O. Activated protein C: biased for translation. Blood 125, 2898-2907 (2015).
6. Polderdijk, S. G. et al. Design and characterization of an APC-specific serpin for the treatment of hemophilia. Blood 129, 105-113 (2017).
7. Escuriola Ettingshausen, C. et al. Symptomatic onset of severe hemophilia A in childhood is dependent on the presence of prothrombotic risk factors. Thromb. Haemost. 85, 218-220 (2001).
8. Vianello, F. et al. Mild bleeding diathesis in a boy with combined severe haemophilia B (C (10400)->T) and heterozygous factor V Leiden. Haemophilia 7, 511-514 (2001).
9. Nichols, W. C. et al. Moderation of hemophilia A phenotype by the factor V R506Q mutation. Blood 88, 1183-1187 (1996).
10. Xu, J. et al. Extracellular histones are major mediators of death in sepsis. Nat. Med. 15, 1318-1321 (2009).
11. Bae, J. S., Yang, L., Manithody, C. & Rezaie, A. R. Engineering a disulfide bond to stabilize the calcium-binding loop of activated protein C eliminates its anticoagulant but not its protective signaling properties. J. Biol. Chem. 282, 9251-9259 (2007).
12. Mosnier, L. O., Gale, A. J., Yegneswaran, S. & Griffin, J. H. Activated protein C variants with normal cytoprotective but reduced anticoagulant activity. Blood 104, 1740-1744 (2004).
13. Mosnier, L. O. et al. Hyperantithrombotic, noncytoprotective Glu149Ala activated protein C mutant. Blood 113, 5970-5978 (2009).
14. Lyden et al., Ann Neural 85:125 (2019).
15. Mann, H. J., Short, M. A. & Schlichting, D. E. Protein C in critical illness. Am. J. Health Syst. Pharm. 66, 1089-1096 (2009).
16. Ranieri, V M et al, N Engl J Med 366:2055 (2012).
17. Angus, DC, Crit Care 16:107 (2012).
18. Expert Opin Biol Ther. 13:1149 (2013).
19. Appl Microbial Biotechnol. 77:13 (2007).
20. Klarenbeek A, et al. Mabs-Austin.; 7:693 (2015).
21. Trends Biochem Sci. 26:230 (2001).
22. Van Audenhove I, Gettemans J. EBioMedicine 8:40 (2016).
23. Van de Water J A, et al. Proc Natl Acad Sci USA. 109:16642 (2012).

1. An isolated antibody comprising a heavy chain variable region (V_{H}) comprising a CDR3 comprising an amino acid sequence selected from the group consisting of SEQ ID NOs: 9 and 118, wherein said antibody specifically binds to activated protein C (APC) and minimally binds to unactivated protein C (PC).

2. The antibody of claim 1, wherein the V_{H} further comprises (a) a CDR1 comprising an amino acid sequence selected from the group consisting of SEQ ID NOs: 1-4 and 109-113, (b) a CDR2 comprising an amino acid sequence selected from the group consisting of SEQ ID NOs: 5-8 and 114-117, or (c) both a CDR1 comprising an amino acid sequence selected from the group consisting of SEQ ID NOs: 1-4 and 109-113 and a CDR2 comprising an amino acid sequence selected from the group consisting of SEQ ID NOs: 5-8 and 114-117.

3. The antibody of claim 2, wherein the V_{H} comprises the CDR1, CDR2, and CDR3 of a V_{H} having an amino acid sequence selected from the group consisting of SEQ ID NOs: 10-15 and 119-129.

4. An isolated antibody comprising a V_{H} comprising an amino acid sequence selected from the group consisting of SEQ ID NOs: 10-15 and 119-129, wherein said antibody specifically binds to APC and minimally binds to PC.

5. An isolated antibody comprising a light chain variable region (V_{L}) comprising a CDR3 comprising an amino acid sequence selected from the group consisting of SEQ ID NOs: 23 and 138-140, wherein said antibody specifically binds to APC and minimally binds to PC.

6. The antibody of claim 5, wherein the V_{L} further comprises (a) a CDR1 comprising an amino acid sequence selected from the group consisting of SEQ ID NOs: 16-19 and 130-131, (b) a CDR2 comprising an amino acid sequence selected from the group consisting of SEQ ID NOs: 21-22 and 132-137, or (c) both a CDR1 comprising an amino acid sequence selected from the group consisting of SEQ ID NOs: 16-19 and 130-131 and a CDR2 comprising an amino acid sequence selected from the group consisting of SEQ ID NOs: 21-22 and 132-137.

7. The antibody of claim 6, wherein the V_{L} comprises the CDR1, CDR2, and CDR3 of a V_{L} having an amino acid sequence selected from the group consisting of SEQ ID NOs: 24-35 and 141-151.

8. An isolated antibody comprising a V_{L} comprising an amino acid sequence selected from the group consisting of SEQ ID NOs: 24-35 and 141-151, wherein said antibody specifically binds to APC and minimally binds to PC.

9. The antibody of claim 5 further comprising a V_{H} comprising a CDR3 comprising an amino acid sequence selected from the group consisting of SEQ ID NOs: 9 and 118.

10. The antibody of claim 9, wherein the V_{H} further comprises (a) a CDR1 comprising an amino acid sequence selected from the group consisting of SEQ ID NOs: 1-4 and 109-113, (b) a CDR2 comprising an amino acid sequence selected from the group consisting of SEQ ID NOs: 5-8 and 114-117, or (c) both a CDR1 comprising an amino acid sequence selected from the group consisting of SEQ ID NOs: 1-4 and 109-113 and a CDR2 comprising an amino acid sequence selected from the group consisting of SEQ ID NOs: 5-8 and 114-117.

11. The antibody of claim 10, wherein the V_{H} comprises the CDR1, CDR2, and CDR3 of a V_{H} having an amino acid sequence selected from the group consisting of SEQ ID NOs: 10-15 and 119-129.

12. The antibody of claim 10, wherein the V_{H} comprises an amino acid sequence selected from the group consisting of SEQ ID NOs: 10-15 and 119-129.

13. An isolated antibody comprising:
1) the V_{H} of SEQ ID NO: 10 and the V_{L} of SEQ ID NO: 24;
2) the V_{H} of SEQ ID NO: 11 and the V_{L} of SEQ ID NO: 25;
3) the V_{H} of SEQ ID NO: 11 and the V_{L} of SEQ ID NO: 26;
4) the V_{H} of SEQ ID NO: 11 and the V_{L} of SEQ ID NO: 27;
5) the V_{H} of SEQ ID NO: 11 and the V_{L} of SEQ ID NO: 28;
6) the V_{H} of SEQ ID NO: 11 and the V_{L} of SEQ ID NO: 29;
7) the V_{H} of SEQ ID NO: 11 and the V_{L} of SEQ ID NO: 30;
8) the V_{H} of SEQ ID NO: 11 and the V_{L} of SEQ ID NO: 31;
9) the V_{H} of SEQ ID NO: 11 and the V_{L} of SEQ ID NO: 32;
10) the V_{H} of SEQ ID NO: 11 and the V_{L} of SEQ ID NO: 33;
11) the V_{H} of SEQ ID NO: 12 and the V_{L} of SEQ ID NO: 28;
12) the V_{H} of SEQ ID NO: 13 and the V_{L} of SEQ ID NO: 34;
13) the V_{H} of SEQ ID NO: 14 and the V_{L} of SEQ ID NO: 35;
14) the V_{H} of SEQ ID NO: 14 and the V_{L} of SEQ ID NO: 34;
15) the V_{H} of SEQ ID NO: 14 and the V_{L} of SEQ ID NO: 28;
16) the V_{H} of SEQ ID NO: 14 and the V_{L} of SEQ ID NO: 31;
17) the V_{H} of SEQ ID NO: 14 and the V_{L} of SEQ ID NO: 32;
18) the V_{H} of SEQ ID NO: 14 and the V_{L} of SEQ ID NO: 33;
19) the V_{H} of SEQ ID NO: 15 and the V_{L} of SEQ ID NO: 28;
20) the V_{H} of SEQ ID NO: 15 and the V_{L} of SEQ ID NO: 29;
21) the V_{H} of SEQ ID NO: 14 and the V_{L} of SEQ ID NO: 29;
22) the V_{H} of SEQ ID NO: 15 and the V_{L} of SEQ ID NO: 33;
23) the V_{H} of SEQ ID NO: 119 and the V_{L} of SEQ ID NO: 29;
24) the V_{H} of SEQ ID NO: 120 and the V_{L} of SEQ ID NO: 29;
25) the V_{H} of SEQ ID NO: 121 and the V_{L} of SEQ ID NO: 29;
26) the V_{H} of SEQ ID NO: 122 and the V_{L} of SEQ ID NO: 29;
27) the V_{H} of SEQ ID NO: 11 and the V_{L} of SEQ ID NO: 141;
28) the V_{H} of SEQ ID NO: 11 and the V_{L} of SEQ ID NO: 142;
29) the V_{H} of SEQ ID NO: 11 and the V_{L} of SEQ ID NO: 143;
30) the V_{H} of SEQ ID NO: 11 and the V_{L} of SEQ ID NO: 144;
31) the V_{H} of SEQ ID NO: 123 and the V_{L} of SEQ ID NO: 29;
32) the V_{H} of SEQ ID NO: 124 and the V_{L} of SEQ ID NO: 29;
33) the V_{H} of SEQ ID NO: 125 and the V_{L} of SEQ ID NO: 29;
34) the V_{H} of SEQ ID NO: 11 and the V_{L} of SEQ ID NO: 145;
35) the V_{H} of SEQ ID NO: 126 and the V_{L} of SEQ ID NO: 29;
36) the V_{H} of SEQ ID NO: 11 and the V_{L} of SEQ ID NO: 146;
37) the V_{H} of SEQ ID NO: 11 and the V_{L} of SEQ ID NO: 147;
38) the V_{H} of SEQ ID NO: 11 and the V_{L} of SEQ ID NO: 148;
39) the V_{H} of SEQ ID NO: 11 and the V_{L} of SEQ ID NO: 149;
40) the V_{H} of SEQ ID NO: 127 and the V_{L} of SEQ ID NO: 29;
41) the V_{H} of SEQ ID NO: 11 and the V_{L} of SEQ ID NO: 150;
42) the V_{H} of SEQ ID NO: 11 and the V_{L} of SEQ ID NO: 151;
43) the V_{H} of SEQ ID NO: 128 and the V_{L} of SEQ ID NO: 29; or
44) the V_{H} of SEQ ID NO: 129 and the V_{L} of SEQ ID NO: 29.

14. The antibody of claim 1, wherein the antibody binds to an exosite of APC.

15. The antibody of claim 1, wherein the antibody is an antibody selected from the group consisting of an IgG1 antibody, an IgG2 antibody, an IgG3 antibody, an IgG4 antibody, an IgM antibody, an IgA1 antibody, an IgA2 antibody, a secretory IgA antibody, an IgD antibody, an IgE antibody, and an antigen-binding fragment thereof.

16. The antibody of claim 15, wherein the antibody is an IgG4 antibody.

17. The antibody of claim 1, wherein the antibody:
a) enhances, minimally affects, or inhibits the anticoagulant activity of APC;
b) enhances, minimally affects, or inhibits APC-mediated histone cleavage;
c) enhances, minimally affects, or inhibits the endothelial barrier protective activity of APC;
d) increases, minimally affects, or decreases the plasma half-life of APC; or
e) any combination thereof.

18. An isolated antibody that specifically binds to APC, minimally binds to PC, and competes with the antibody of claim 1 for binding to APC.

19. A nucleic acid encoding the antibody of claim 1.

20. A vector comprising the isolated nucleic acid of claim 19.

21. A host cell comprising the nucleic acid of claim 19.

22. A method of producing an antibody, the method comprising:
a) culturing the host cell of claim 21 under conditions that result in production of the antibody; and
b) isolating the antibody from the host cell.

23. A pharmaceutical composition comprising the antibody of claim 1 and a pharmaceutically acceptable carrier.

24. A method for treating or preventing a condition in a subject in need thereof, the method comprising administering a therapeutically effective amount of the antibody of claim 1 to the subject.

25. The method of claim 24, wherein the condition is a condition that can be treated or prevented by enhancing or inhibiting the anticoagulant function of APC.

26. The method of claim 25, wherein the condition is selected from the group consisting of a hemorrhage, a contusion, a burn, gastrointestinal bleeding, uncontrolled bleeding, bleeding due to a transplantation or resection procedure, bleeding due to a surgery, bleeding due to a traumatic injury, variceal bleeding, bleeding in cirrhosis, thrombocytopenia, idiopathic thrombocytopenia purpura, hemophilia, aortic aneurysm, over-administration of an anticoagulant or antithrombotic, menorrhagia, deficiency of a clotting factor, Glanzmann's Thrombasthenia, and Bernard-Soulier syndrome.

27. The method of claim 24, wherein the condition is a condition that can be treated or prevented by enhancing or inhibiting one or more cytoprotective function of APC.

28. The method of claim 27, wherein the condition is selected from the group consisting of sepsis, inflammation in acute ischemic disease, coronavirus disease 2019 (COVID-19), diabetes, diabetic nephropathy, diabetic ulcers, wounds, amyotrophic lateral sclerosis (ALS), multiple sclerosis, central nervous system injury, ischemic stroke, Alzheimer's disease, acute kidney injury, a lung disorder, acute pancreatitis, a cancer, an inflammatory disease, and an autoimmune disease.

29.-33. (canceled)
